(19) United States
(12) Patent Application Publication
(10) Pub. No.: US 2017/0042819 A1
(43) Pub. Date: Feb. 16, 2017

(54) NANOPARTICLES FOR TARGETED GENE THERAPY AND METHODS OF USE THEREOF

(71) Applicant: Avrygen Corporation, San Francisco, CA (US)

(72) Inventor: Randy Goomer, Millbrae, CA (US)

(21) Appl. No.: 15/305,641

(22) PCT Filed: Apr. 23, 2015
(86) PCT No.: PCT/US15/27389

§ 371 Date: Oct. 20, 2016

Related U.S. Application Data
(60) Provisional application No. 61/983,218, filed on Apr. 23, 2014.

(51) Int. Cl. A61K 9/16; C12N 15/113

(52) U.S. Cl. CPC A61K 9/1658; A61K 9/1641; C12N 15/113; C12N 2310/351; C12N 2310/14; C12N 2310/531; C12N 2310/141

(57) ABSTRACT

The present disclosure provides targeted, polymeric nanoparticles which facilitate the delivery of small interfering RNAs, miRNAs and shRNA expressing plasmid DNAs and include an aggregate of nucleic acids and polycationic polymer scaffolds. Methods of making and using such nanoparticles are provided as are methods of treating cancer, including Glioblastoma Multiforme, prostate cancer and melanoma using such nanoparticles.

### CROSS-REFERENCE

This application claims the benefit of U.S. Provisional Application No. 61/983,218 filed Apr. 23, 2014, the contents of which are incorporated by reference herein in their entirety.

### INTRODUCTION

Significant research effort has gone into the application of RNA interference (RNAi) and the use of interfering RNA (e.g., siRNA, shRNA or miRNA) in the treatment of disease, including cancer. Despite the potential utility of interfering RNA, there are limitations which make clinical application difficult, including issues with delivery, side effects due to off-target actions, and induction of the host immune response. Researchers have attempted to overcome these limitations and to improve the safety and efficacy of potential RNAi-based therapeutics. Nanoparticles (NPs), which are nanostructured entities which may be tailored with respect to size, shape, and surface modification, provide a potential means for addressing some of the above considerations. While attempts to deliver nucleic acids using nanoparticles have been made, there remains a need in the art for additional compositions and methods adapted to successfully deliver gene therapy plasmids, small interfering RNAs (siRNAs), therapeutic micro-RNAs (miRNA) and short hairpin RNA (shRNA) expressing plasmid DNAs, particularly in the field of RNAi-based cancer therapy.

Glioblastoma Multiforme (GBM) is a devastating and ultimately fatal cancer for which most available therapeutic strategies are either ineffective or nonspecific and hence are highly toxic. Current treatment options include surgical resection followed by radiation and chemotherapy. In spite of aggressive treatment, GBM remains a lethal cancer due to its invasive growth infiltrating brain tissue, complex alterations in growth-promoting signaling pathways, and the presence of GBM stem cells (GSC), which contribute to tumor recurrence.

Current treatment of GBM includes chemotherapy with temozolomide (TMZ), which alkylates/methylalates guanine residues in DNA. However, the use of TMZ is accompanied by significant side-effects and does not provide significant survival benefits. Moreover, GBM tumor cells are able to repair this type of DNA damage, thereby diminishing the therapeutic efficacy of TMZ. Even with use of TMZ therapy most patients with GBM perish within 14-months following diagnosis. Thus, targeted therapeutics with high specificity for the most invasive and therapeutically resistant GSC would effectively complement current standard treatment regimen.

Melanoma rates have steadily increased by 60% since 1991 while the mortality rate has not dropped within the same time frame. Currently, $2.9 billion are spent on melanoma treatment. Treatment options for metastatic melanoma are very limited with standard care being single agent chemotherapy combined with surgery; unfortunately, even with aggressive treatment 10 year survival rates are less than 10%.

According to cancer.org website, prostate cancer is one of the most common cancers in American men. The American Cancer Society estimates that there will be 220,800 new cases of prostate cancer and about 27,540 men will die from prostate cancer in the US this year. About 1 in 7 men in the US will be diagnosed with prostate cancer during their lifetime, of these, 1 in 38 will die of prostate cancer, making it the second leading cause of cancer death in American men, behind only lung cancer. While prostate cancer treatment and surveillance has made great strides with awareness and early treatment, once the cancer becomes metastatic the 10-year survival rate drops to 28%.

The present disclosure addresses the above concerns and provides related methods and compositions for the treatment of disease, with particular applicability to the treatment of cancers, such as GBM, melanoma, and prostate cancer.

### SUMMARY OF THE INVENTION

The present disclosure provides targeted, polymeric nanoparticles which facilitate the delivery of small interfering RNAs, miRNAs and shRNA expressing plasmid DNAs and which include an aggregate of nucleic acids and polycationic polymer scaffolds in specific condensed states, forming nanoparticles. Methods of making and using such nanoparticles are provided as are methods of treating cancer, including Glioblastoma Multiforme, prostate cancer and melanoma, using such nanoparticles.

Other aspects and embodiments will be readily apparent to the ordinarily skilled artisan upon reading the present specification.

### BRIEF DESCRIPTION OF THE DRAWINGS

FIG. 1A provides a schematic of a polycationic polymer scaffold according to embodiments of the present disclosure, wherein the polycationic polymer scaffold includes an amphiphilic peptide/target binding moiety covalently bound to the polycationic polymer scaffold, a hydrophilic polymer covalently bound to the polycationic polymer scaffold, and nucleic acids bound by ionic-charge interactions to the polycationic polymer scaffold. In such embodiments, the amphiphilic peptide may function as a target binding moiety and the nucleic acids may include, e.g., interfering RNAs such as siRNA or miRNA or a DNA template driving expression of shRNAs.

FIG. 1B provides a schematic of a polycationic polymer scaffold according to other embodiments of the present disclosure, wherein the polycationic polymer scaffold includes an amphiphilic peptide covalently bound to the polycationic polymer scaffold, a target binding moiety covalently bound to the polycationic polymer scaffold, a hydrophilic polymer covalently bound to the polycationic polymer scaffold, and nucleic acids bound by ionic-charge interactions to the polycationic polymer scaffold. In such embodiments, the nucleic acids may include, e.g., interfering RNAs such as siRNA or miRNA or a DNA template driving expression of shRNAs.

FIG. 1C provides a schematic of a polycationic polymer scaffold according to other embodiments of the present disclosure, wherein the polycationic polymer scaffold includes an amphiphilic peptide/target binding moiety covalently bound to the polycationic polymer scaffold, a hydrophilic polymer covalently bound to the polycationic polymer scaffold, a blood brain barrier (BBB) transport moiety covalently bound to the polycationic polymer scaffold, and nucleic acids bound by ionic-charge interactions to the polycationic polymer scaffold. In such embodiments, the amphiphilic peptide may function as a target binding moiety and the nucleic acids may include, e.g., interfering RNAs such as siRNA or miRNA or a DNA template driving expression of shRNAs.

FIG. 1D provides a schematic of a polycationic polymer scaffold according to other embodiments of the present disclosure, wherein the polycationic polymer scaffold includes an amphiphilic peptide/target binding moiety covalently bound to the polycationic polymer scaffold, a hydrophilic polymer covalently bound to the polycationic polymer scaffold, a BBB transport moiety covalently bound to the polycationic polymer scaffold, a detectable label covalently bound to the polycationic polymer scaffold, and nucleic acids bound by ionic-charge interactions to the polycationic polymer scaffold. In such embodiments, the amphiphilic peptide may function as a target binding moiety and the nucleic acids may include, e.g., interfering RNAs such as siRNA or miRNA or a DNA template driving expression of shRNAs.

FIG. 1E provides a schematic of a more specific embodiment of the polycationic polymer scaffold depicted in FIG. 1D, including chlorotoxin (ClTx) as an amphiphilic peptide/target binding moiety covalently bound to the polycationic polymer scaffold, polyethylene glycol (PEG) as the hydrophilic polymer covalently bound to the polycationic polymer scaffold, and a transferrin receptor ligand as the BBB transport moiety covalently bound to the polycationic polymer scaffold.

FIG. 1F provides a schematic depicting condensation of nucleic acids (e.g., siRNA, miRNA, or plasmid driving expression of shRNA or miRNA) by polycationic polymer containing covalently bound BBB (e.g., Transferrin or ClTx), hydrophilic polymer (e.g., PEG), and amphiphilic peptide (e.g., ClTx) according to an embodiment of the present disclosure. A. depicts interaction of a nucleic acid monomer (i.e., a NP nucleic acid molecule subunit) with a covalently modified polycationic polymer. B. depicts several nucleic acid monomers (i.e., NP nucleic acid molecule subunits) condensed with polycationic polymers into a nanoparticle (NP) with hydrophilic (HP), amphiphilic (AP, also referred to herein as AmP), and blood-brain-barrier (BBB) transport moieties on the nanoparticle surface.

FIG. 1G provides a schematic depicting condensation of nucleic acids (e.g., DNA or RNA) by, e.g., polycationic polymer-bound BBB transport moiety (e.g., polycationic polymer-bound Transferrin (TPL)), polycationic polymer-bound amphiphilic peptide/target binding moiety (e.g., polycationic polymer-bound ClTx (CPL)), polycationic polymer-bound hydrophilic polymer (e.g., polycationic polymer-bound PEG (PPL)), polycationic polymer-bound amphiphilic peptide, e.g., polycationic polymer-bound Am1 (AmPL)), a copolymer of a polycationic polymer and a hydrophobic polymer (which copolymer is referred to herein as (PLPL)), e.g., a copolymer of poly-lysine and lyso-phosphatidylethanolamine, a copolymer of a polycationic polymer, e.g., poly-lysine, and polyethylenimine (PEI) (LXEI), and polycationic polymer-bound fluorescent label, e.g., polycationic polymer-bound Cy5.5 fluorescent label (CyPL) or polycationic polymer-bound rhodamine (RPL), according to an embodiment of the present disclosure.

FIG. 1H provides a schematic depicting condensation of nucleic acids (e.g., DNA or RNA) by, e.g., polycationic polymer-bound Transferrin (TPL), polycationic polymer-bound ClTx (CPL), polycationic polymer-bound PEG (PPL), polycationic polymer-bound amphiphilic peptide Am1 (AmPL), a copolymer of a polycationic polymer and a hydrophobic polymer (PLPL), a copolymer of a polycationic polymer, e.g., poly-lysine, and polyethylenimine (PEI) (LXEI), and polycationic polymer-bound fluorescent label, e.g., polycationic polymer-bound Cy5.5 fluorescent label (CyPL) or polycationic polymer-bound rhodamine (RPL), according to an embodiment of the present disclosure. Specific percentages for the various components are provided.

FIG. 1I provides a schematic depicting condensation of nucleic acids (e.g., plasmid driving expression of shRNA or miRNA) by polycationic polymer with covalently bound BBB transport moiety (e.g., Transferrin or ClTx), hydrophilic polymer (e.g., PEG), and amphiphilic peptide, e.g., to target tumors of neuroectodermal origin, (e.g., ClTx or Am1). A. depicts interaction of a nucleic acid monomer (i.e., a NP nucleic acid molecule subunit) with covalently modified polycationic polymer. B. depicts several nucleic acid monomers (i.e., NP nucleic acid molecule subunits) condensed with polycationic polymers into a nanoparticle (NP) with hydrophilic (HP), amphiphilic (AP), and blood-brain-barrier (BBB) transport moieties on the nanoparticle surface.

FIG. 2 U87 primary Glioblastoma cells were transfected using 10⁷ final form formulated NP in complete media. Light microscopy image (left panel) and fluorescent image (right panel) at 488 nm excitation at GFP specific filter are shown 48-hours after delivery.

FIG. 3 provides images of primary GSC cultures in a murine model of GBM. (Panel A) Primary GBM tissues were sorted for stem cell markers and cultured in neurosphere conditions in a defined media (left-phase photomicrograph; right-immunofluorescent detection of Nestin and CD133 (two stem cell markers)). (Panel B) Immuno-fluorescence analysis of primary GSC cells shows they are positive for Id-1 (middle) and nestin (right). Control IgG-left. Cells were counterstained with DAPI. (Panel C) H&E staining of intracranially grown tumors derived from primary GSC. (Panel D) High magnification demonstrates its histological resemblance to human GBM. (Panel E) Luciferase labeled GSC1 cells were injected in nude mice at two cell densities. Tumor growth was monitored in real time using the IVIS Lumina instrument and tumor size and survival were recorded.

FIG. 4 In-vivo results where ClTx provides tissue specific targeting of nanoparticles to GBM and inhibition of Sox2 expression in GBM murine model following i.v. delivery of nanoparticles. (Panels A and B). Seventy two hours after i.v. delivery of nanoparticles, mice were monitored by whole body luminescence scanning for Cy5.5 signal. Tissue distribution following delivery of ClTx-Cy5.5-Tf-PEG-PL_{235/}Sox2 siRNA (Panel A, mouse on the right) was directly compared with the same nanoparticle using control siRNA (Panel A, left mouse). Luminescence measurements are shown in Panel B. Brain specific nanoparticle delivery was prominent in the presence of ClTx. (Panels C, D and E) Imaging of the brain, kidneys and liver following nanoparticle delivery. (Panels F and G) Seventy-two hours following i.v. delivery of nanoparticles, brains were flash frozen and processed for immunofluorescence for Sox2. Representative photomicrographs are shown. Panel F—control siRNA-NP, Panel G—Sox2 siRNA-NP. Counterstaining with DAPI. Bar=100 μm.

FIG. 5 Shows results of Example 5 demonstrating gene delivery in Prostate cells. GFP gene delivery was performed in Panels A1-A3: using NP1 (containing: TPL (6.78E+13); LXEI (1.06E+14); PLPL (2.82E+13); AmPL (5.65E+13); RPL (8.48E+12) and PPL (4.24E+13)) and in Panels B1-B3 using NP2 (containing: TPL (6.78E+13); LXEI (1.06E+14); PLPL (2.82E+13); AmPL (1.06E+14); RPL (8.48E+12) and PPL (4.24E+13)) condensed with 1.24 μg pGFP plasmid DNA. Microscopy was performed using light (Bright Field), green fluorescence (GFP) and red fluorescence (RHO).

FIG. 6 provides a graph showing that PPL containing NPs produce higher pDNA Uptake than free PEG as described in Example 6.

FIG. 7 provides a graph showing GFP expression by Taqman® analysis as described in Example 6.

FIG. 8 provides fluorescence microscopy images showing gene delivery to primary brain neurons as described in Example 7.

FIG. 9 provides a graph showing a reduction in BPTF expression in primary human melanoma cells following treatment with NP carrying BPTF specific siRNA as described in Example 8.

FIG. 10 provides a graph showing that CPL containing NP significantly enhanced NP gene delivery and expression in the brain after i.v. injections as described in Example 9. RNA was isolated from brain tissues and used for Taqman® real-time PCR quantification of GFP expression, normalized to the house keeping human RPL13A gene. Y-axis shows normalized GFP expression which is x-fold over control. Two mice per group were used. CPL containing NP injected i.v., via tail produced robust brain specific GFP expression.

FIG. 11 shows prostate harvested and dissected away from the bladder, imaged in Zeiss stereo Lumar for GFP expression and analyzed using Zen pro 2012 software module as described in Example 10.

FIG. 12 provides a graph showing tissue distribution of GFP expression following NP delivery of GFP plasmid as described in Example 10.

FIG. 13 shows neurospheres imaged by bright field (BF) (image 1 and 4), green fluorescence (GF; image 2 and 5) and red fluorescence (RF; image 3 and 6) microscopy as described in Example 11. NP was covalently conjugated to rhodamine (R) red fluorescent dye and the formulated NP were loaded with either plasmid driving expression of cop-GFP gene (pGFP) or FAM dye labeled 21-nt RNA marker (FAM-RNA) that concentrates and fluoresces when located inside the cell nucleus. Neurospheres were imaged 72 hours after delivery.

FIG. 14 provides images from of live luminescence and fluorescence imaging of a nude mouse bearing an intracranial GBM and injected with 3× of CPL-CyPL-TPL-PPL-AmPL-LXEI-PLPL/pGFP, (NP16) delivering (3.72 μg) GFP plasmid DNA as described in Example 11. Mice were injected twice at 72 hr interval, delivering 3.72 μg DNA each time. The image on left is showing tumor cell luminescence with luciferin, while the image on the right shows biodistribution of NPs containing Cy5.5 label as CyPL conjugate. Radiant fluorescence was visualized by excitation at 675 nm and using emission filter specific for Cy5.5 label.

FIG. 15 provides images and graphs demonstrating in vivo delivery and functional efficacy of NPs in GBM-bearing nude mouse as described in Example 11. Panels (A) and (D) Left panels: Luminescence measurements to visualize the xenografted GBM tumors. Right panels: Fluorescence image of NP16 carrying mir-34a: CPL-CyPL-TPL-PPL-AmPL-LXEI-PLPL/mir-34a (A) and NP16 carrying mir-128 CPL-CyPL-TPL-PPL-AmPL-LXEI-PLPL/mir-128 (D) shows distribution of the NP within the brain tumor. Panels (B) and (E): Taqman measurements of miR-34a and mir-128 delivery to GBM. Brain tissue (the tumor containing hemisphere) from two mice/group was used to extract RNA. Control mice were injected with the same NP formulation delivering random miRNA. Mice were treated twice every 72 h, and each i.v. injection delivered 3.72 μg miRNA, establishing a safe and functionally effective NP dose. Panel (C): Western blot was performed on protein isolated from the same tissues as shown in B. Smad4, ID1 and Actin levels were measured in the control and miRNA34a treated samples. Western analysis demonstrated pronounced and specific inhibition of the downstream protein targets of mir-34a in vivo (Panel (C)).

FIG. 16 ACTX-01a provides an image and graph showing the successful delivery of active CD44 siRNA into tumor bearing nude mice brain as described in Example 12. Specifically, ACTX-01a (3×NP14 containing 8.93E+12 (3% TPL; 1.06E+14 (32%) LXEI; 2.82E+13 (9%) PLPL; 8.93E+13 (27%) AmPL; 1.87E+13 (6%) CyPL; 6.33E+13 (19%) PPL, and 1.70E+13 (5%) CPL used to condense 1.24 μg CD44 siRNA) was injected via tail vein in tumor bearing nude mice. The CD44 specific gene expression in these mice was specifically down-regulated by over 49%.

FIG. 17 provides images showing that ACTX-01a reduced primary human tumor growth in a mouse model of the human disease as described in Example 12. PDX model was started by intracranial injection of GSC3832 in nude mice. On day 13, the mice were imaged for luminescence to determine tumor size and injected with ACTX-01a. On day 17 mice received a second injection of ACTX-01a and were imaged for luminescence. On day 21 the tumor had significantly shrunk in mice receiving ACTX-01a.

FIG. 18 provides a chemical formula showing a polycationic polymer conjugated to 1 or 2 “X” moieties formed with a phosphate headgroup and a long- or short-chain hydrophobic region. In X, n can range, for example, from 1 to 30.

FIG. 19 provides a chemical formula showing the LXEI copolymer where PEI is covalently bound to a polycationic polymer, e.g., poly-lysine, with a PEI:PL ratio of, e.g., 1:1 to 4:1. In this structure X can range, for example, from 70 to 235 or more and Y can range, for example, from 10 to 32 or more.

### DEFINITIONS

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials are now described. All publications and applications mentioned herein are incorporated by reference to disclose and describe the methods and/or materials in connection with which the publications are cited. To the extent any of the applications or publications incorporated by reference herein conflict with the instant disclosure, the instant disclosure controls.

It must be noted that as used herein and in the appended claims, the singular forms “a”, “and”, and “the” include plural referents unless the context clearly dictates otherwise. Thus, for example, reference to “a polymeric nanoparticle” includes a plurality of such polymeric nanoparticles and reference to the “polycationic polymer scaffold” includes reference to one or more polycationic polymer scaffolds and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As used herein, the term “active agent” means an agent, e.g., a protein, peptide, nucleic acid (including, e.g., nucleotides, nucleosides and analogues thereof) or small molecule drugs, that provides a desired pharmacological effect upon administration to a subject, e.g., a human or a non-human animal, either alone or in combination with other active or inert components. Included in the above definition are precursors, derivatives, analogues and prodrugs of active agents.

The term “CPL” is used herein to refer to a chlorotoxin (ClTx) covalently bound to a polycationic polymer scaffold.

The term “PPL” is used herein to refer to a polyethylene glycol (PEG) covalently bound to a polycationic polymer scaffold.

The term “TPL” is used herein to refer to a transferrin receptor ligand (e.g., Transferrin) covalently bound to a polycationic polymer scaffold.

The term “AmPL” is used herein to refer to an amphiphilic peptide Am1 covalently bound to a polycationic polymer scaffold.

The term “CyPL” is used herein to refer to a Cy5.5 fluorescent label covalently bound to a polycationic polymer scaffold.

The term “RPL” is used herein to refer to a rhodamine label covalently bound to a polycationic polymer scaffold.

The terms “peptide”, “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The terms include fusion proteins, including, but not limited to, fusion proteins with a heterologous amino acid sequence, fusions with heterologous and native leader sequences, with or without N-terminal methionine residues; immunologically tagged proteins; fusion proteins with detectable fusion partners, e.g., fusion proteins including as a fusion partner a fluorescent protein, 0-galactosidase, luciferase, etc.; and the like.

The terms “antibody” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins including an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. Also encompassed by the terms are Fab′, Fv, F(ab′)₂, and other antibody fragments that retain specific binding to antigen.

Antibodies may exist in a variety of other forms including, for example, Fv, Fab, and (Fab′)₂, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., Immunology, Benjamin, N.Y., 2nd ed. (1984), and Hunkapiller and Hood, Nature, 323, 15-16 (1986).

The terms “nucleic acid”, “nucleic acid molecule” and “polynucleotide” are used interchangeably and refer to a polymeric form of nucleotides of any length, either deoxyribonucleotides or ribonucleotides, or analogs thereof. The terms encompass, e.g., DNA, RNA and modified forms thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

“RNA interference” (RNAi) is a process by which double-stranded RNA (dsRNA) is used to silence gene expression. Without intending to be bound by any particular theory, RNAi begins with the cleavage of longer dsRNAs into small interfering RNAs (siRNAs) by dicer, an RNaseIII-like enzyme. siRNAs are dsRNAs that are generally about 19 to 28 nucleotides, or 20 to 25 nucleotides, or 21 to 23 nucleotides in length and often contain 2-3 nucleotide 3′ overhangs, and 5′ phosphate and 3′ hydroxyl termini. One strand of the siRNA is incorporated into a ribonucleoprotein complex known as the RNA-induced silencing complex (RISC). RISC uses this siRNA strand to identify mRNA molecules that are at least partially complementary to the incorporated siRNA strand, and then cleaves these target mRNAs or inhibits their translation. The siRNA strand that is incorporated into RISC is known as the guide strand or the antisense strand. The other siRNA strand, known as the passenger strand or the sense strand, is eliminated from the siRNA and is at least partially homologous to the target mRNA. Those of skill in the art will recognize that, in principle, either strand of an siRNA can be incorporated into RISC and function as a guide strand. However, siRNA may be designed (e.g., via decreased siRNA duplex stability at the 5′ end of the antisense strand) to favor incorporation of the antisense strand into RISC.

RISC-mediated cleavage of mRNAs having a sequence at least partially complementary to the guide strand leads to a decrease in the steady state level of that mRNA and of the corresponding protein encoded by the mRNA. Alternatively, RISC can also decrease expression of the corresponding protein via translational repression without cleavage of the target mRNA. Other RNA molecules can interact with RISC and silence gene expression. Examples of other RNA molecules that can interact with RISC include short hairpin RNAs (shRNAs), single-stranded siRNAs, microRNAs (miRNAs), and dicer-substrate 27-mer duplexes, RNA molecules containing one or more chemically modified nucleotides, one or more deoxyribonucleotides, and/or one or more non-phosphodiester linkages. The term “siRNA” as used herein refers to a double-stranded interfering RNA unless otherwise noted. For purposes of the present discussion, all RNA molecules that can interact with RISC and participate in RISC-mediated changes in gene expression will be referred to as “interfering RNAs”. siRNAs, shRNAs, miRNAs, and dicer-substrate 27-mer duplexes are, therefore, subsets of interfering RNAs.

As used herein, the term “target binding moiety” refers to a molecule having a specific binding affinity for a target, e.g., a target molecule, such as a target protein, wherein such target is other than a polynucleotide that binds to the target binding moiety through a mechanism which predominantly depends on Watson/Crick base pairing. Exemplary target binding moieties include, e.g., receptors, receptor ligands, antibodies, antigens, aptamers, and binding fragments thereof. In some embodiments, the affinity between a target binding moiety and a target when they are specifically bound to each other is characterized by a KD (dissociation constant) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M.

As used herein, the terms “specifically binds,” “binds specifically,” and the like refer to an interaction between binding partners such that the binding partners bind to one another, but do not bind other molecules that may be present in the environment (e.g., in a biological sample, in tissue) at a significant or substantial level under a given set of conditions (e.g., physiological conditions).

As used herein, “fluorescent group” refers to a molecule that, when excited with light having a selected wavelength, emits light of a different wavelength. Fluorescent groups may also be referred to as “fluorophores”.

As used herein, the terms “determining,” “measuring,” “assessing,” and “assaying” are used interchangeably and include both quantitative and qualitative determinations.

As used herein the term “isolated,” when used in the context of an isolated compound, refers to a compound of interest that is in an environment different from that in which the compound naturally occurs. “Isolated” is meant to include compounds that are within samples that are substantially enriched for the compound of interest and/or in which the compound of interest is partially or substantially purified.

As used herein, the term “substantially pure” refers to a compound that is removed from its natural environment and is at least 60% free, 75% free, or 90% free from other components with which it is naturally associated.

A “coding sequence” or a sequence that “encodes” a selected polypeptide, is a nucleic acid molecule which is transcribed (in the case of DNA) and translated (in the case of mRNA) into a polypeptide, for example, in-vivo when placed under the control of appropriate regulatory sequences (or “control elements”). The boundaries of the coding sequence are typically determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and synthetic DNA sequences. A transcription termination sequence may be located 3′ to the coding sequence. Other “control elements” may also be associated with a coding sequence. A DNA sequence encoding a polypeptide can be optimized for expression in a selected cell by using the codons preferred by the selected cell to represent the DNA copy of the desired polypeptide coding sequence.

“Encoded by” refers to a nucleic acid sequence which codes for a gene product, such as a polypeptide. Where the gene product is a polypeptide, the polypeptide sequence or a portion thereof contains an amino acid sequence of at least 3 to 5 amino acids, 8 to 10 amino acids, or at least 15 to 20 amino acids from a polypeptide encoded by the nucleic acid sequence. The term “encoded by” may also be used herein to refer to an RNA transcript of a DNA sequence, e.g., an shRNA transcript of a DNA sequence.

“Operably linked” refers to an arrangement of elements wherein the components so described are configured so as to perform their usual function. In the case of a promoter, a promoter that is operably linked to a coding sequence will have an effect on the expression of a coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

By “nucleic acid construct” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes including non-native nucleic acid sequences, and the like.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells, which can be accomplished by genomic integration of all or a portion of the vector, or transient or inheritable maintenance of the vector as an extrachromosomal element. Thus, the term includes cloning, and expression vehicles, as well as integrating vectors. The term “vector” may also be used herein to refer to a nucleic acid construct capable of directing the expression of an RNA of interest, e.g., the expression of an shRNA from a plasmid vector.

An “expression cassette” includes any nucleic acid construct capable of directing the expression of a gene/coding sequence of interest or RNA of interest, which is operably linked to a promoter of the expression cassette. Such cassettes can be constructed into a “vector,” “vector construct,” “expression vector,” or “gene transfer vector,” in order to transfer the expression cassette into target cells. Thus, the term includes cloning and expression vehicles, as well as viral vectors.

Techniques for determining nucleic acid and amino acid “sequence identity” are known in the art. Typically, such techniques include determining the nucleotide sequence of the mRNA for a gene and/or determining the amino acid sequence encoded thereby, and comparing these sequences to a second nucleotide or amino acid sequence. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics, 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986).

An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the internet address located by placing http:// in front of blast.ncbi.nlm.nih.gov/Blast.cgi.

Alternatively, in the context of polynucleotides, homology can be determined by hybridization of polynucleotides under conditions that form stable duplexes between homologous regions, followed by digestion with single-stranded-specific nuclease(s), and size determination of the digested fragments.

Two nucleic acid, or two polypeptide sequences are “substantially homologous” to each other when the sequences exhibit at least about 80%-85%, at least about 85%-90%, at least about 90%-95%, or at least about 95%-98% or greater sequence identity over a defined length of the molecules, as determined using the methods above. As used herein, substantially homologous also refers to sequences showing complete identity to the specified nucleic acid or polypeptide sequence. Nucleic acid sequences that are substantially homologous can be identified in a Southern hybridization experiment under, for example, stringent conditions, as defined for that particular system. Defining appropriate hybridization conditions is within the skill of the art. See, e.g., Sambrook and Russel, Molecular Cloning: A Laboratory Manual Third Edition, (2001) Cold Spring Harbor Laboratory Press, Cold Spring Harbor, N.Y.

A first polynucleotide is “derived from” a second polynucleotide if it has the same or substantially the same nucleotide sequence as a region of the second polynucleotide, its cDNA, complements thereof, or if it displays sequence identity as described above. This term is not meant to require or imply the polynucleotide must be obtained from the origin cited (although such is encompassed), but rather can be made by any suitable method.

A first polypeptide (or peptide) is “derived from” a second polypeptide (or peptide) if it is (i) encoded by a first polynucleotide derived from a second polynucleotide, or (ii) displays sequence identity to the second polypeptides as described above. This term is not meant to require or imply the polypeptide must be obtained from the origin cited (although such is encompassed), but rather can be made by any suitable method.

The term “in combination with” as used herein refers to uses where, for example, a first therapy is administered during the entire course of administration of a second therapy; where the first therapy is administered for a period of time that is overlapping with the administration of the second therapy, e.g. where administration of the first therapy begins before the administration of the second therapy and the administration of the first therapy ends before the administration of the second therapy ends; where the administration of the second therapy begins before the administration of the first therapy and the administration of the second therapy ends before the administration of the first therapy ends; where the administration of the first therapy begins before administration of the second therapy begins and the administration of the second therapy ends before the administration of the first therapy ends; where the administration of the second therapy begins before administration of the first therapy begins and the administration of the first therapy ends before the administration of the second therapy ends. As such, “in combination” can also refer to regimen involving administration of two or more therapies. “In combination with” as used herein also refers to administration of two or more therapies which may be administered in the same or different formulations, by the same or different routes, and in the same or different dosage form type.

The terms “treatment”, “treating”, “treat”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine.

“Subject”, “host” and “patient” are used interchangeably herein, to refer to an animal, human or non-human, amenable to therapy according to the methods of the disclosure or to which a polymeric nanoparticle composition according to the present disclosure may be administered to achieve a desired effect. Generally, the subject is a mammalian subject.

As used herein, the term “nanoparticle” refers to a particle having at least one dimension, e.g., diameter or length, of from about 1 nm to about 100 nm.

As used herein, the term “aggregate” refers to a particle composed of nucleic acids and polycationic polymers held together via charged-based interactions between the nucleic acids and polycationic polymers, wherein the hydrodynamic size of the nucleic acids is reduced as a result of the interactions.

### DETAILED DESCRIPTION OF EXEMPLARY EMBODIMENTS OF THE INVENTION

The disclosure is directed to targeted, polymeric nanoparticles which facilitate the delivery of interfering RNA and include an aggregate of nucleic acids and polycationic polymer scaffolds. Methods of making and using such nanoparticles are provided as are methods of treating cancer, including Glioblastoma Multiforme (GBM), melanoma and prostate cancer, using such nanoparticles. Generally, a polymeric nanoparticle according to the present disclosure includes aggregates of nucleic acids and polycationic polymer scaffolds, wherein the aggregates includes a polycationic polymer scaffold, an amphiphilic peptide covalently bound to the polycationic polymer scaffold, a hydrophilic polymer covalently bound to the polycationic polymer scaffold, and a nucleic acid bound by ionic-charge interactions to the polycationic polymer scaffold. In some embodiments the amphiphilic peptide also functions as a target binding moiety. In these embodiments, as well as other embodiments, an additional target binding moiety may be included which may or may not be amphiphilic. In some embodiments, the aggregate includes a blood brain barrier (BBB) transport moiety (e.g., Transferrin or ClTx) covalently bound to the polycationic polymer scaffold forming TPL or CPL. In some embodiments, the BBB transport moiety also functions as a target binding moiety. The BBB transport moiety, amphiphilic peptide, hydrophilic polymer, and/or target binding moiety may be provided as individual NP polymer scaffold subunits, with each component covalently bonded to a distinct polycationic polymer scaffold molecule. Alternatively, or in addition, two or more of these components may be provided covalently bonded to one or more polycationic polymer scaffold molecules used to form the NP.

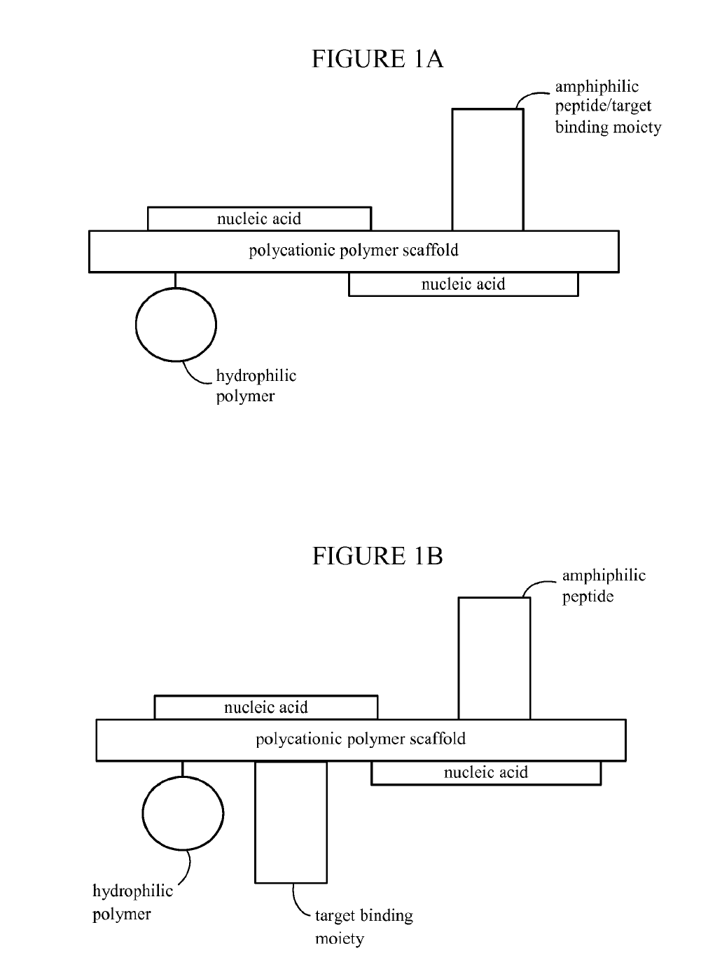

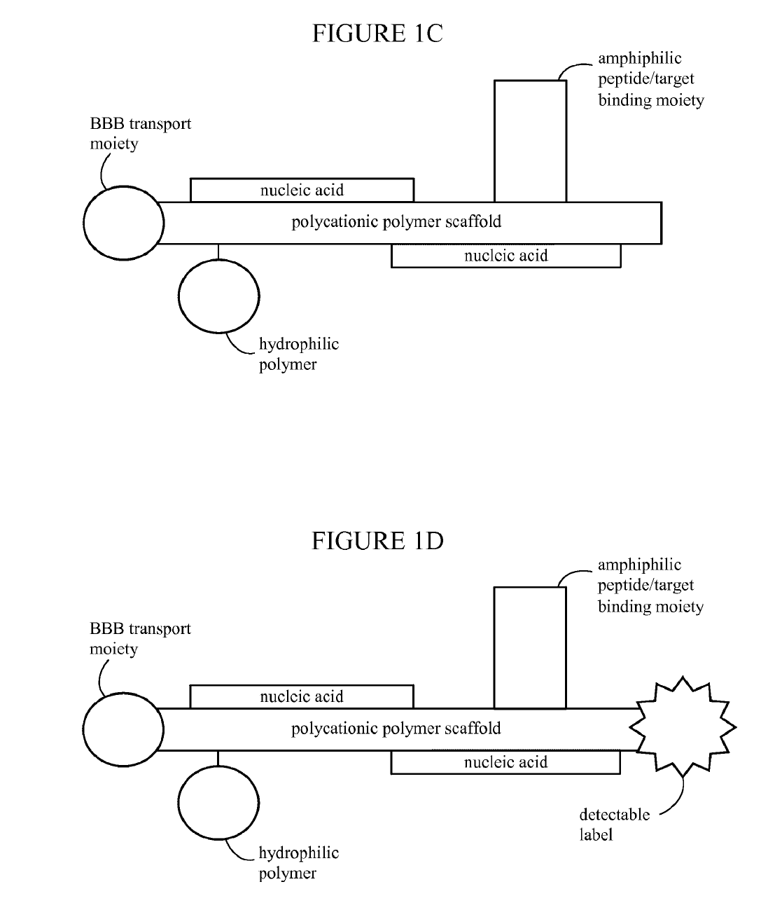

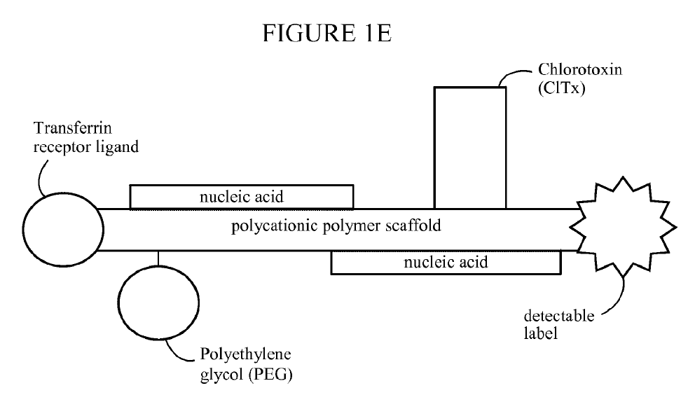

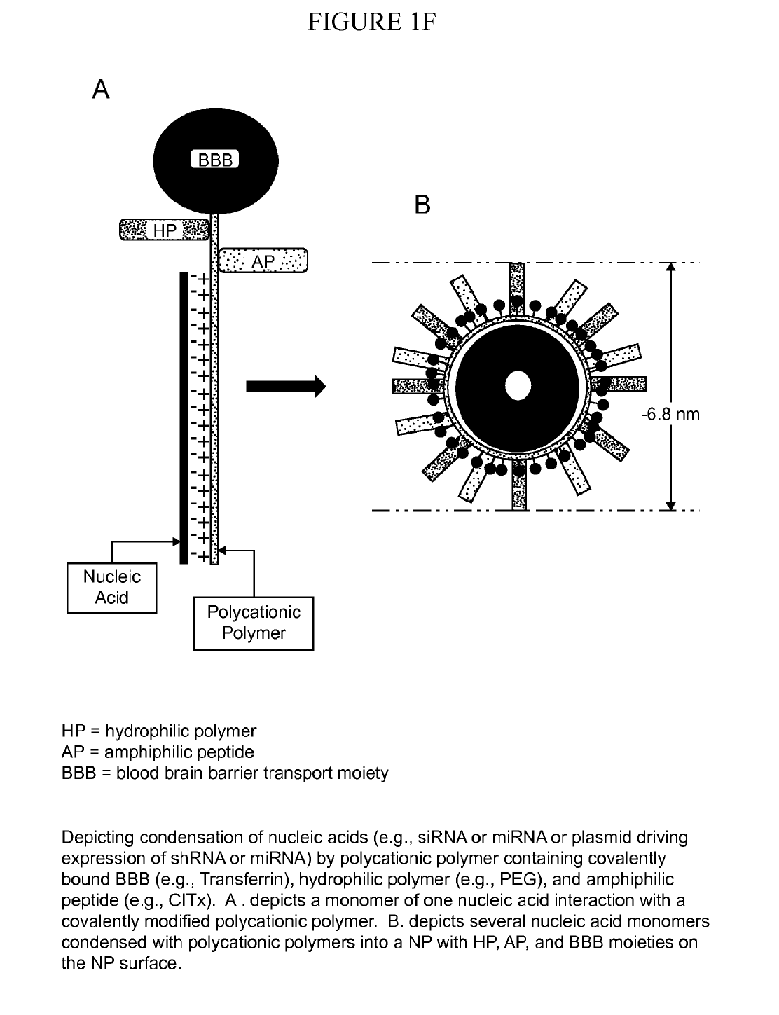

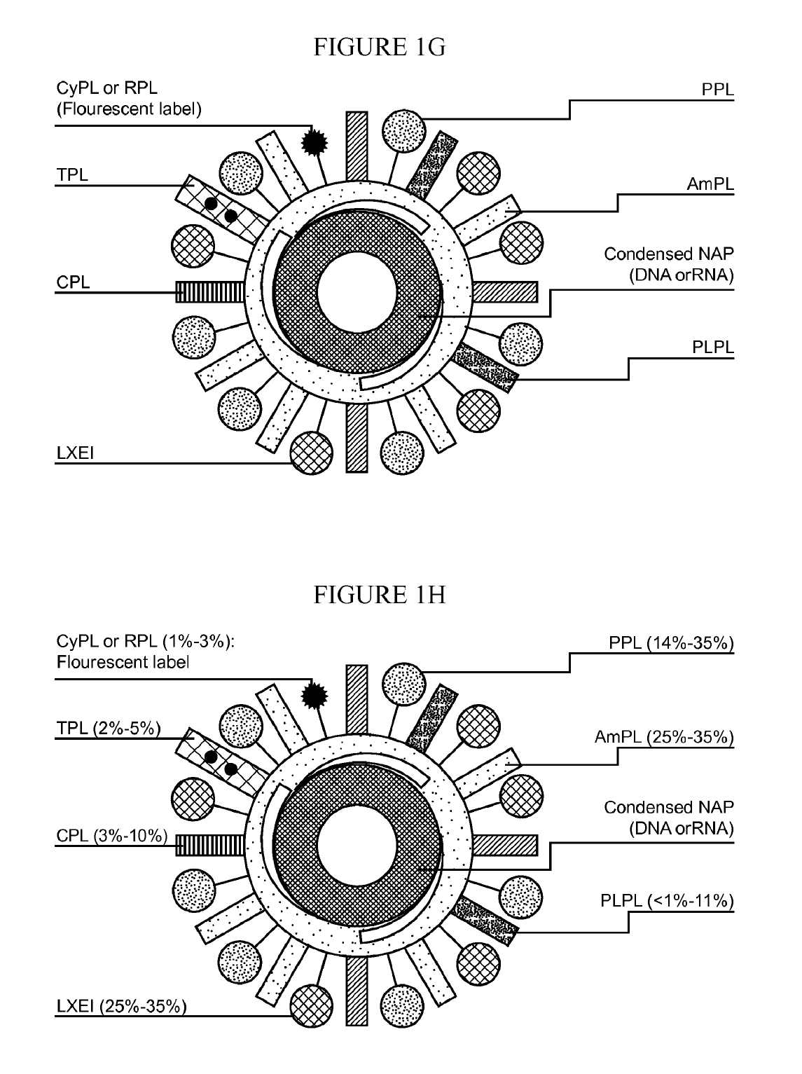

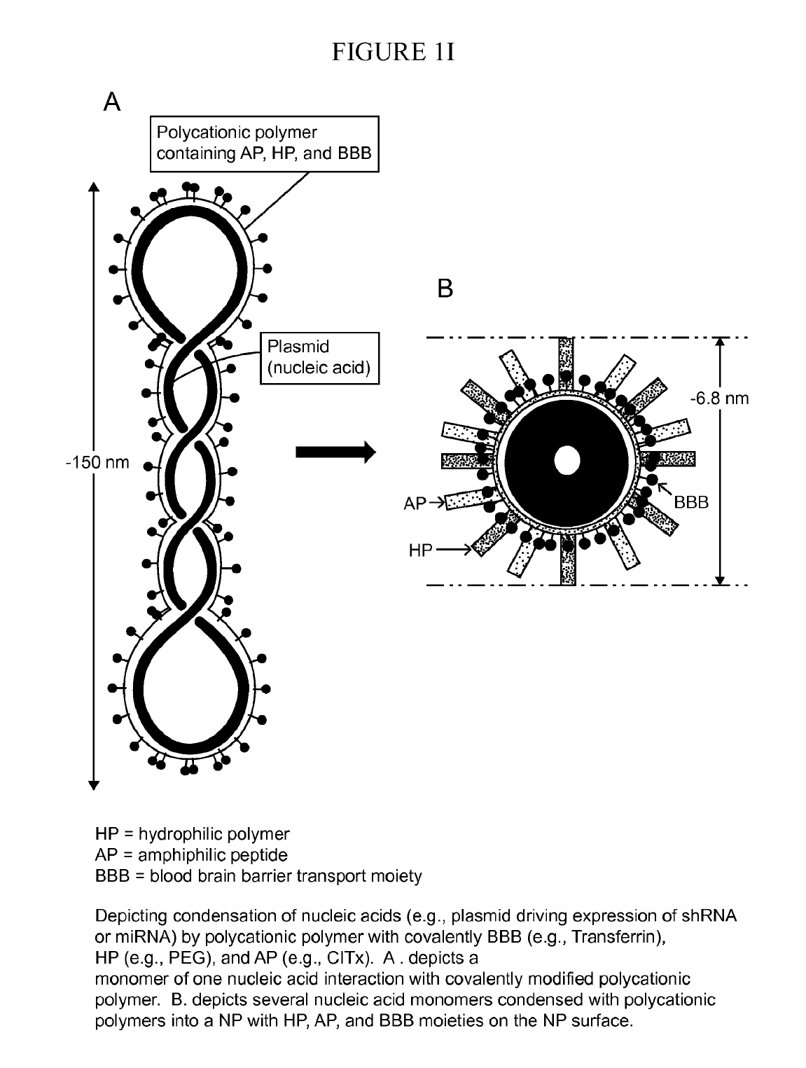

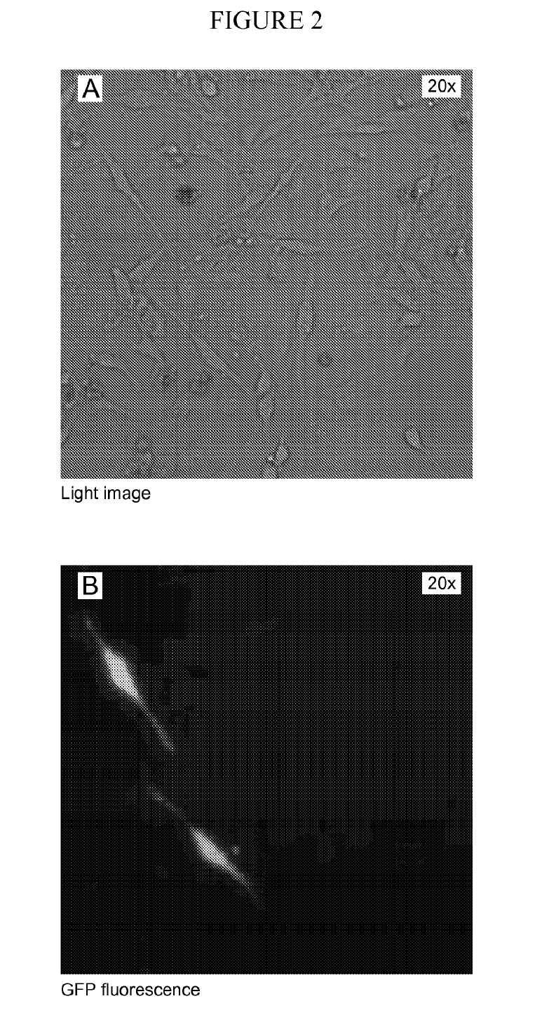

Generalized schematics of covalently-modified polycationic polymer scaffolds according to embodiments of the present disclosure are provided in FIGS. 1A-1I. FIG. 1A depicts a polycationic polymer scaffold which has been covalently modified with an amphiphilic peptide which may optionally function as a target binding moiety. The polycationic polymer scaffold is also covalently modified with a hydrophilic polymer. Finally, FIG. 1A depicts a plurality of nucleic acid molecules bound by ionic-charge interactions to the polycationic polymer scaffold.

FIG. 1B depicts a polycationic polymer scaffold which has been covalently modified with an amphiphilic peptide and a separate target binding moiety. The polycationic polymer scaffold is also covalently modified with a hydrophilic polymer. Finally, FIG. 1B depicts a plurality of nucleic acid molecules bound by ionic-charge interactions to the polycationic polymer scaffold.

FIG. 1C depicts a polycationic polymer scaffold which has been covalently modified with an amphiphilic peptide which may optionally function as a target binding moiety. The polycationic polymer scaffold is also covalently modified with a hydrophilic polymer and a BBB transport moiety. Finally, FIG. 1C depicts a plurality of nucleic acid molecules bound by ionic-charge interactions to the polycationic polymer scaffold.

FIG. 1D depicts a polycationic polymer scaffold which has been covalently modified with an amphiphilic peptide which may optionally function as a target binding moiety. The polycationic polymer scaffold is also covalently modified with a hydrophilic polymer, a BBB transport moiety, and a detectable label. Finally, FIG. 1D depicts a plurality of nucleic acid molecules bound by ionic-charge interactions to the polycationic polymer scaffold.

FIG. 1E depicts a more specific embodiment of a covalently modified polycationic polymer scaffold, wherein chlorotoxin (ClTx) or a derivative thereof is covalently attached to the polycationic polymer scaffold as the BBB transport moiety or amphiphilic peptide, a polyethylene glycol (PEG) is covalently attached to the polycationic polymer scaffold as the hydrophilic polymer, a transferrin receptor ligand, e.g., transferrin, is covalently attached to the polycationic polymer scaffold as the BBB transport moiety, and a detectable label is covalently attached to the polycationic polymer scaffold. Finally, FIG. 1E depicts a plurality of nucleic acid molecules bound by ionic-charge interactions to the polycationic polymer scaffold.

FIGS. 1F-1I provide schematics depicting condensation of nucleic acids by covalently-modified polycationic polymers according to the present disclosure as well as nanoparticles formed thereby.

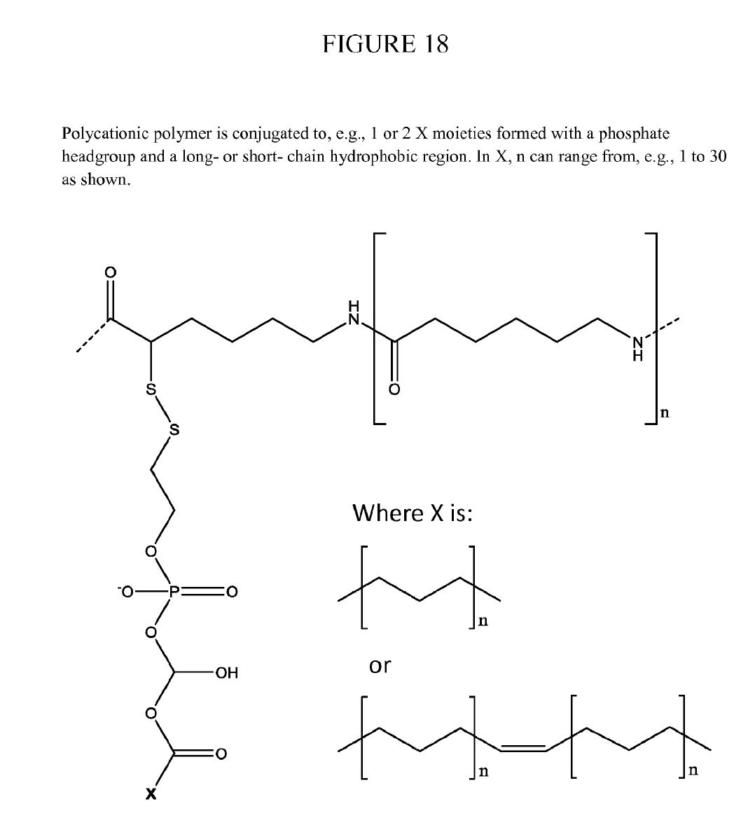

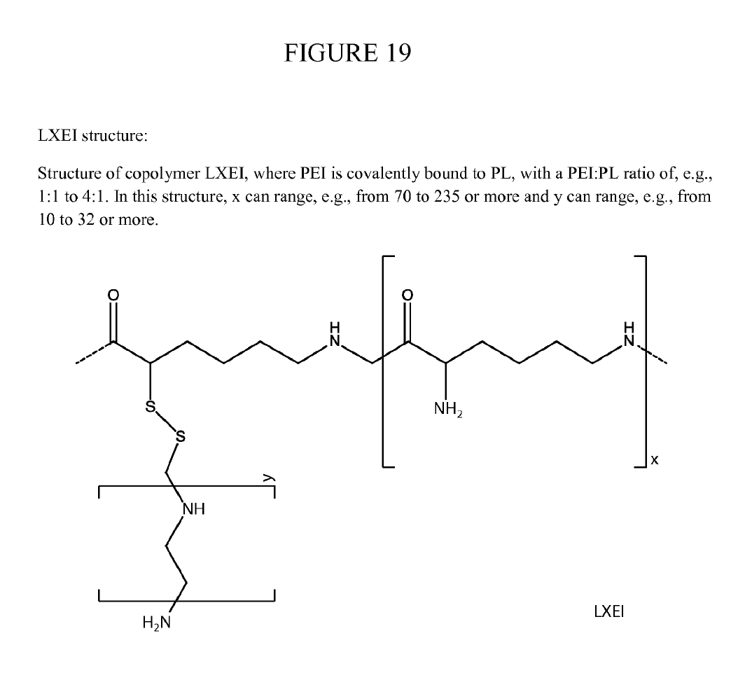

FIG. 1G provides a schematic depicting condensation of nucleic acids (e.g., DNA or RNA) by polycationic polymer-bound BBB transport moiety (e.g., polycationic polymer-bound Transferrin (TPL)), polycationic polymer-bound amphiphilic peptide/target binding moiety (e.g., polycationic polymer-bound ClTx (CPL)), polycationic polymer-bound hydrophilic polymer (e.g., polycationic polymer-bound PEG (PPL)), polycationic polymer-bound amphiphilic peptide, e.g., polycationic polymer-bound Am1 (AmPL)), a copolymer of a polycationic polymer and a hydrophobic polymer (which copolymer is referred to herein as (PLPL)), e.g., a poly-lysine conjugated to lyso-phosphatidylethanolamine, and polycationic polymer-bound label, e.g., fluorescent label, e.g., polycationic polymer-bound Cy5.5 fluorescent label (CyPL) or polycationic polymer-bound rhodamine (RPL), according to an embodiment of the present disclosure. The NP may also include a copolymer of a polycationic polymer, e.g., poly-lysine, and polyethylenimine (PEI) (LXEI). In some embodiments, each of the above components is provided as a distinct polycationic polymer-bound NP subunit, wherein each NP subunit is covalently bonded to a distinct polycationic polymer molecule and a plurality of the NP subunits covalently bonded to distinct polycationic polymer molecules are used to condense nucleic acids (e.g., DNA or RNA) into an NP according to the present disclosure. In other embodiments, two or more of the above NP subunits are covalently bonded to a single polycationic polymer molecule and a group of such covalently-modified polycationic polymer molecules are used either alone or in combination with one or more NP subunits covalently bonded to distinct polycationic polymer molecules to condense nucleic acids (e.g., DNA or RNA) into an NP.

Where a nanoparticle according to the present disclosure includes a copolymer of a polycationic polymer and a hydrophobic polymer (which copolymer is referred to herein as (PLPL)), e.g., a poly-lysine (PL) conjugated to lyso-phosphatidylethanolamine, the polycationic polymer can be conjugated, for example, to 1 or 2 “X” moieties formed with a phosphate headgroup and a long- or short-chain hydrophobic region as shown in FIG. 18, wherein, for X, n can range, e.g., from 1 to 30.

Where a nanoparticle according to the present disclosure includes a copolymer of a polycationic polymer, e.g., poly-lysine (PL), and polyethylenimine (PEI) (LXEI), such a copolymer can have the chemical structure set forth in FIG. 19, wherein the PEI:PL ratio is, e.g., 1:1 to 4:1. In this structure X can range, for example, from 70 to 235 or more and Y can range, for example, from 10 to 32 or more.

In some embodiments, polycationic polymer-bound BBB transport moiety (e.g., polycationic polymer-bound Transferrin (TPL)) accounts for 2% to 12% of the monomers (i.e., NP polymer scaffold subunits) (e.g., 4% to 12%, 6% to 12%, 8% to 12%, or 10% to 12%; or 4% to 10%, such as 6% to 8%) which make up the NP (i.e., the polycationic polymer-bound BBB transport moiety is present at a molar amount of 2% to 12% relative to the total moles of NP polymer scaffold subunits present in the NP); polycationic polymer-bound amphiphilic peptide/target binding moiety (e.g., polycationic polymer-bound ClTx (CPL)) accounts for 3% to 10% of the monomers (i.e., NP polymer scaffold subunits) (e.g., 4% to 10%, 6% to 10% or 8% to 10%; or 4% to 8%, such as 6%) which make up the NP (i.e., the polycationic polymer-bound ClTx is present at a molar amount of 3% to 10% relative to the total moles of NP polymer scaffold subunits present in the NP); polycationic polymer-bound hydrophilic polymer (e.g., polycationic polymer-bound PEG (PPL)) accounts for 14% to 35% of the monomers (i.e., NP polymer scaffold subunits) (e.g., 15% to 35%, 20% to 35%, or 30% to 35%; or 15% to 30%, such as 20% to 25%) which make up the NP (i.e., the polycationic polymer-bound hydrophilic polymer is present at a molar amount of 14% to 35% relative to the total moles of NP polymer scaffold subunits present in the NP); polycationic polymer-bound amphiphilic peptide, e.g., polycationic polymer-bound Am1 (AmPL)) accounts for 25% to 35% of the monomers (i.e., NP polymer scaffold subunits) (e.g., 26% to 35%, 28% to 35%, 30% to 35%, 32% to 35% or 34% to 35%; or 26% to 33%, such as 28% to 31%, such as 30%) which make up the NP (i.e., the polycationic polymer-bound amphiphilic peptide is present at a molar amount of 25% to 35% relative to the total moles of NP polymer scaffold subunits present in the NP); a copolymer of a polycationic polymer and a hydrophobic polymer (PLPL) e.g., a copolymer of poly-lysine and lyso-phosphatidylethanolamine, accounts for <1% to 11% of the monomers (i.e., NP polymer scaffold subunits) (e.g., 1% to 11%, 2% to 11% 4% to 11%, 6% to 11%, 8% to 11%, or 10% to 11%; or 2% to 10%, such as 4% to 8%, such as 6%) which make up the NP (i.e., the copolymer of a polycationic polymer and a hydrophobic polymer is present at a molar amount of <1% to 11% relative to the total moles of NP polymer scaffold subunits present in the NP); and polycationic polymer-bound label, e.g., fluorescent label, e.g., polycationic polymer-bound Cy5.5 fluorescent label (CyPL) or polycationic polymer-bound rhodamine (RPL) accounts for 1% to 3% of the of the monomers (i.e., NP polymer scaffold subunits) (e.g., 1.5% to 3%, 2% to 3%, or 2.5% to 3%; or 1.5% to 2.5%, such as 2%) which make up the NP (i.e., the polycationic polymer-bound label is present at a molar amount of 1% to 3% relative to the total moles of NP polymer scaffold subunits present in the NP). The NP may also include a copolymer of a polycationic polymer, e.g., poly-lysine (PL), and polyethylenimine (PEI) (LXEI), wherein LXEI accounts for 25% to 35% of the of the monomers (i.e., NP polymer scaffold subunits) (e.g., 26% to 35%, 28% to 35%, 30% to 35%, 32% to 35%, or 34% to 35%; or 26% to 34%, such as 28% to 32%, such as 30%) which make up the NP (i.e., the copolymer of a polycationic polymer and polyethylenimine is present at a molar amount of 25% to 35% relative to the total moles of NP polymer scaffold subunits present in the NP).

It should be noted that in particular embodiments, one or more of the above NP polymer scaffold subunits may not be present in a NP according to the present disclosure.

A more detailed description of the polymeric nanoparticles of the present disclosure and the covalently-modified polycationic polymer scaffolds of the present disclosure, as well as their various components is provided below.

Polymeric Nanoparticles

The polymeric nanoparticles provided by the present disclosure provide a means for delivering nucleic acids, such as interfering RNA, within specific cells and/or tissue types. The polymeric nanoparticles of the present disclosure are composed of aggregates of nucleic acids and covalently-modified polycationic polymer scaffolds. As discussed in greater detail below, the polycationic polymer scaffolds which aggregate with nucleic acids to form the polymeric nanoparticles of the present disclosure are generally covalently modified with at least one amphiphilic peptide, at least one target binding moiety, and at least one hydrophilic polymer. In some embodiments, the amphiphilic peptide may also function as the target binding moiety, in which case the inclusion of an additional target binding moiety is optional. The amphiphilic peptide, hydrophilic polymer, and/or target binding moiety may be provided as individual NP polymer scaffold subunits, with each component covalently bonded to a distinct polycationic polymer scaffold molecule. Alternatively, or in addition, two or more of these components may be provided covalently bonded to one or more polycationic polymer scaffold molecules used to form the NP.

The polymeric nanoparticles of the present disclosure generally have at least one dimension (e.g., diameter or length) of from about 1 nm to about 100 nm, e.g., from about 1 nm to about 90 nm, from about 1 nm to about 80 nm, from about 1 nm to about 70 nm, from about 1 nm to about 60 nm, from about 1 nm to about 50 nm, from about 1 nm to about 40 nm, from about 1 nm to about 30 nm, from about 1 nm to about 20 nm, or from about 1 nm to about 10 nm.

In some embodiments, a polymeric nanoparticle according to the present disclosure has at least one dimension (e.g., diameter or length) of from about 1 nm to about 4 nm, about 4 nm to about 8 nm, or from about 8 nm to about 12 nm.

The polymeric nanoparticles of the present disclosure may be provided in any suitable shape, with spheroidal and toroidal nanoparticles being of particular interest.

The polymeric nanoparticles of the present disclosure may include a variety of suitable materials as discussed in greater detail below. The polymeric nanoparticles of the present disclosure may be characterized as having a core including aggregates of nucleic acids and covalently-modified polycationic polymer scaffolds as described herein. In some embodiments, the polymeric nanoparticles of the present disclosure may be characterized as including aggregates as described herein, wherein the aggregates are distributed generally homogenously throughout the polymeric nanoparticles, e.g., so as to provide a matrix of such aggregates. As such the polymeric nanoparticles of the present disclosure do not require, and in some embodiments specifically exclude, metallic and/or magnetic materials.

Polycationic Polymer Scaffolds

Polycationic polymer scaffolds which find use in the disclosed nanoparticle compositions allow for the non-covalent, charged-based binding of one or more nucleic acids to the polycationic polymer scaffolds. Without intending to be bound by any particular theory, it is proposed that these polycationic polymer scaffolds facilitate the condensation of nucleic acid molecules and the formation of nanoparticle structures by interaction of the polycationic polymer scaffolds with the nucleic acid molecules, whereby the negative charges on the back-bone phosphates of the nucleic acid molecules are neutralized. This condensation dramatically reduces the hydrodynamic diameter of the nucleic acids generally forming nanoparticles with spherical or toroidal geometry.

A variety of suitable polycationic polymers which may be utilized as the polycationic polymer scaffolds of the present disclosure are known in the art, including, e.g., synthetic polycationic polymers, such as poly-lysine (e.g., poly-L-lysine), poly-arginine, poly-glutamine, poly-amine, polyethylenimine (PEI), poly(diallyldimethylammonium chloride) (pDADMAC), cyclodextrin-based polycation, synthetic polymers with conjugated positive charge moieties; and naturally occurring polymers, such as chitosan (or molecules related thereto or derived therefrom) and atelocollagen.

Polycationic polymers may also advantageously cause the accumulation of ions in the low pH environment of endosomes where delivered nucleic acids may be sequestered producing a so-called “proton-sponge” effect which results in endosomal burst and subsequent nucleic acid escape into the cytoplasm.

Amphiphilic Peptides

Amphiphilic peptides which find use in the disclosed nanoparticle compositions generally facilitate cellular uptake of the nanoparticles and subsequent release of the nanoparticle-associated nucleic acids into the cytosol. Such peptides may also contribute to the “proton-sponge” effect discussed above which may facilitate endosomal burst and subsequent nucleic acid escape into the cytoplasm.

A variety of suitable amphiphilic peptides which may be conjugated to the polycationic polymer scaffolds of the present disclosure are known in the art, including, e.g., amphiphilic peptides belonging to the Pep-1, MPG and CADY families. See, e.g., Morris et al. Biol. Cell (2008) 100:201-217. Of particular interest in the present disclosure is the Chlortoxin (ClTx) peptide (e.g., the ClTx peptide having the following amino acid sequence: MCMPCFTTDHQMARKCDDCCGGKGRGKCYGPQCLCR) which is both an amphiphilic peptide which contributes to endosomal burst and a target binding moiety which can target the nanoparticles to cancer cells such as GBM stem cells by specifically binding such cells and facilitating preferential delivery of NP to brain by effectively crossing the BBB.

In some embodiments, a suitable amphiphilic peptide conjugated to a polycationic polymer scaffold is a polycationic polymer-bound ClTx (CPL). In some embodiments, a CPL accounts for 3% to 10% of the monomers (i.e., NP polymer scaffold subunits) (e.g., 4% to 10%, 6% to 10% or 8% to 10%; or 4% to 8%, such as 6%) which make up a NP according to the present disclosure (i.e., the polycationic polymer-bound ClTx is present at a molar amount of 3% to 10% relative to the total moles of NP polymer scaffold subunits present in the NP).

Also of interest as an amphiphilic peptide for the covalent modification of the polycationic polymer scaffolds of the present disclosure is the bee venom derived peptide melittin and derivatives or modified versions thereof. Of particular interest is a peptide (Am1) having the following amino acid sequence: NH2-GIGAVLKVLTTGLPALISWIKRKRHHC-CO₂H, e.g., an Am1 peptide bound to a polycationic scaffold, e.g., a PL₂₃₅ scaffold, forming AmPL.

In some embodiments, a suitable amphiphilic peptide conjugated to a polycationic polymer scaffold is a polycationic polymer-bound Am1 peptide (AmPL). In some embodiments, an AmPL accounts for 25% to 35% of the monomers (i.e., NP polymer scaffold subunits) (e.g., 26% to 35%, 28% to 35%, 30% to 35%, 32% to 35% or 34% to 35%; or 26% to 33%, such as 28% to 31%, such as 30%) which make up a NP according to the present disclosure (i.e., the AmPL is present at a molar amount of 25% to 35% relative to the total moles of NP polymer scaffold subunits present in the NP).

A polycationic polymer scaffold according to the present disclosure may be covalently modified with one or more of the above amphiphilic peptides or one or more peptides which are substantially homologous to one of the above amphiphilic peptides. For example, a polycationic polymer scaffold according to the present disclosure may be covalently modified with one or more amphiphilic peptides having at least about 80% amino acid sequence identity with one of the amphiphilic peptides discussed above, e.g., at least about 85% sequence identity, at least about 90% sequence identity, at least about 95% sequence identity, at least about 98% sequence identity, or at least about 99% sequence identity with one of the amphiphilic peptides discussed above. In some embodiments, a polycationic polymer scaffold according to the present disclosure may be covalently modified with one or more amphiphilic peptides having from about 80% to about 99% amino acid sequence identity, e.g., from about 85% to about 99%, from about 90% to about 99%, from about 95% to about 99%, or from about 98% to about 99% sequence identity with one of the amphiphilic peptides discussed above.

In some embodiments, a polycationic polymer scaffold covalently conjugated with an amphiphilic peptide accordingly to the present disclosure includes a polycationic polymer scaffold and an amphiphilic peptide in a molar ratio of from about 1:1 to about 1:10, e.g., about 1:1 to about 1:9, about 1:1 to about 1:8, about 1:1 to about 1:7, about 1:1 to about 1:6, about 1:1 to about 1:5, about 1:1 to about 1:4, about 1:1 to about 1:3, or about 1:1 to about 1:2.

Target Binding Moieties

Target binding moieties which finds use in the disclosed nanoparticle compositions generally provide for targeted delivery of a nucleic acid-containing polymeric nanoparticle according to the present disclosure to a specific cell and/or tissue type, e.g., via cell surface receptor interaction. A target binding moiety according to the present disclosure is a molecule having a specific binding affinity for a target, e.g., a target molecule, and may include any of a variety of known peptides or nucleic acids, which are capable of specifically binding a target, e.g., a protein target, of interest. For example, a suitable target binding moiety may provide a ligand-receptor binding interaction when brought into contact with its corresponding receptor or ligand. Target proteins for which such target binding moieties are known in the art include, e.g., cell surface receptors. Exemplary target binding moieties include, e.g., receptors, ligands, antibodies, antigens, nucleic acid aptamers, and the like.

In some embodiments, a suitable target binding moiety includes a full length antibody or an antibody fragment containing an antigen binding domain, antigen binding domain fragment or an antigen binding fragment of the antibody (e.g., an antigen binding domain of a single chain) which is capable of specifically binding, to a target of interest, usually a protein target of interest.

Of particular interest in the present disclosure is the Chlortoxin (ClTx) peptide which, as discussed above, is both an amphiphilic peptide, which contributes to endosomal burst, and a target binding moiety which can target the nanoparticles to cancer cells such as GBM stem cells by specifically binding such cells, e.g., by binding to matrix metalloproteinase 2 (MMP-2) on the surface of such cells.

In some embodiments, a suitable target binding moeity conjugated to a polycationic polymer scaffold is a polycationic polymer-bound ClTx (CPL). In some embodiments, a CPL accounts for 3% to 10% of the monomers (i.e., NP polymer scaffold subunits) (e.g., 4% to 10%, 6% to 10% or 8% to 10%; or 4% to 8%, such as 6%) which make up a NP according to the present disclosure (i.e., the CPL is present at a molar amount of 3% to 10% relative to the total moles of NP polymer scaffold subunits present in the NP).

A polycationic polymer scaffold according to the present disclosure may be modified with one or more of the above target binding moieties or one or more molecules which are substantially homologous to one of the above target binding moieties. For example, a polycationic polymer scaffold according to the present disclosure may be covalently modified with one or more target binding moieties having at least about 80% amino acid sequence identity with one of the target binding moieties discussed above, e.g., at least about 85% sequence identity, at least about 90% sequence identity, at least about 95% sequence identity, at least about 98% sequence identity, or at least about 99% sequence identity with one of the target binding moieties discussed above. In some embodiments, a polycationic polymer scaffold according to the present disclosure may be covalently modified with one or more target binding moieties having from about 80% to about 99% amino sequence identity, e.g., from about 85% to about 99%, from about 90% to about 99%, from about 95% to about 99%, or from about 98% to about 99% sequence identity with one of the target binding moieties discussed above.

In the case of homologs of chlorotoxin, in some embodiments, amino acid sequence identity of less than 80% (e.g., from about 50% to about 70%) may be tolerated while maintaining the desired activity, provided the 4 c-c disulfide bridges along with the amino acid charges are maintained. In native chlorotoxin, 4 c-c disulfide bridges are present between Cys2-Cys19, Cys5-Cys28, Cys16-Cys33 and Cys20-Cys35. In addition, amino acid sequence identity of less than 80% may be tolerated as long as the peptide's ability to block calcium ion activated chloride ion (Cl-) channels is maintained at comparable levels to native chlorotoxin.

In addition, where the polycationic polymer scaffold is a peptide, such as a poly-lysine, poly-arginine, or poly-glutamine, a target binding moiety may be conjugated to the N-terminal, the C-terminal or both the N- and C-terminal of the peptide.

In some embodiments, a polycationic polymer scaffold covalently conjugated with a target binding moiety accordingly to the present disclosure includes a polycationic polymer scaffold and a target binding moiety in a molar ratio of from about 1:1 to about 1:10, e.g., about 1:1 to about 1:9, about 1:1 to about 1:8, about 1:1 to about 1:7, about 1:1 to about 1:6, about 1:1 to about 1:5, about 1:1 to about 1:4, about 1:1 to about 1:3, or about 1:1 to about 1:2.

Blood Brain Barrier (BBB) Transport Moieties

Blood brain barrier (BBB) transport moieties which finds use in the disclosed nanoparticle compositions generally provide for transport of a nucleic acid-containing polymeric nanoparticle according to the present disclosure across the BBB, e.g., via transporter mediated transcytosis. As with the amphiphilic peptide, hydrophilic polymer, and/or target binding moiety, the BBB transport moiety may be provided as an individual NP polymer scaffold subunit, with the BBB transport moiety covalently bonded to a distinct polycationic polymer scaffold molecule. Alternatively, or in addition, a BBB transport moieties may be provided covalently bonded to one or more polycationic polymer scaffold molecules including one or more of an amphiphilic peptide, hydrophilic polymer, and/or target binding moiety covalently bonded thereto.

In some embodiments, a BBB transport moiety as described herein also functions as a target binding moiety, providing for targeted delivery of a nucleic acid-containing polymeric nanoparticle according to the present disclosure to a specific cell and/or tissue type. A variety of BBB transport moieties, which are capable of facilitating transport across the BBB, and which may find use in the disclosed nanoparticle compositions, are known in the art. For example, transporter mediated transcytosis via targeting of the transferrin receptor can be achieved using the endogenous ligand transferrin or by using antibodies directed against the transferrin receptor. In some embodiments, apo-transferrin is utilized as the BBB transport moiety. Another transferrin which may be utilized in connection with the disclosed nanoparticle compositions is the RNA transferrin described in Wilner S E, et al. “An RNA alternative to human transferrin: A new tool for targeting human cells.” Molecular therapy—Nucleic acids, (2012) 1, e21, the disclosure of which is incorporated by reference herein.

In some embodiments, a suitable BBB transport moiety conjugated to a polycationic polymer scaffold is a polycationic polymer-bound Transferrin (TPL). In some embodiments, a TPL accounts for 2% to 12% of the monomers (i.e., NP polymer scaffold subunits) (e.g., 4% to 12%, 6% to 12%, 8% to 12%, or 10% to 12%; or 4% to 10%, such as 6% to 8%) which make up a NP according to the present disclosure (i.e., the TPL is present at a molar amount of 2% to 12% relative to the total moles of NP polymer scaffold subunits present in the NP).

Suitable BBB transport moieties may include, e.g., amyloid beta peptide or its fragments, ApoE, ApoJ, alpha2-macroglobulin; transthyretin and albumin and antibodies against these molecules. Molecules that interact with receptor for advanced end glycation products (RAGE) may also be used.

Additionally, transport across the BBB may be achieved via targeting of the insulin receptor, e.g., by using monoclonal antibodies directed against the insulin receptor. The low-density lipoprotein receptor related proteins 1 and 2 (LRP-1 and 2) may also be targeted in a manner similar to the transferrin receptor and the insulin receptor to facilitate transport across the BBB. Finally, non-toxic mutants of diphtheria toxin may be utilized as a targeting mechanism for delivery across the BBB.

In some embodiments, chlorotoxin (ClTx) also functions as a BBB transport moiety.

A polycationic polymer scaffold according to the present disclosure may be modified with one or more of the above BBB transport moieties or one or more molecules which are substantially homologous to one of the above BBB transport moieties. For example, a polycationic polymer scaffold according to the present disclosure may be covalently modified with one or more BBB transport moieties having at least about 80% amino acid sequence identity with one of the BBB transport moieties discussed above, e.g., at least about 85% sequence identity, at least about 90% sequence identity, at least about 95% sequence identity, at least about 98% sequence identity, or at least about 99% sequence identity with one of the BBB transport moieties discussed above. In some embodiments, a polycationic polymer scaffold according to the present disclosure may be covalently modified with one or more BBB transport moieties having from about 80% to about 99% amino acid sequence identity, e.g., from about 85% to about 99%, from about 90% to about 99%, from about 95% to about 99%, or from about 98% to about 99% sequence identity with one of the BBB transport moieties discussed above.

In the case of homologs of transferrin, in some embodiments an amino acid sequence identity of less than 80%, e.g., an amino acid sequence idenity of from about 30% to about 80%, e.g., about 30% to about 70%, about 30% to about 60%, about 30% to about 50%, or about 30% to about 40%, may be tolerated while maintaining the desired activity, provided that the ligand binds transferrin receptor, i.e., as long as the docking moiety is present and functional.

In addition, where the polycationic polymer scaffold is a peptide, such as a poly-lysine, poly-arginine, or poly-glutamine, a BBB transport moiety may be conjugated to the N-terminal, the C-terminal or both the N- and C-terminal of the peptide.

In some embodiments, a polycationic polymer scaffold covalently conjugated with a BBB transport moiety accordingly to the present disclosure includes a polycationic polymer scaffold and a BBB transport moiety in a molar ratio of from about 1:1 to about 1:5, e.g., 1:1 to 1:4, 1:1 to 1:3, or 1:1 to 1:2.

Hydrophilic Polymers

Hydrophilic polymers which find use in the disclosed nanoparticle compositions generally provide or contribute to one or more of the following: steric stabilization, evasion of the host immune system, and protection against the effects of the surrounding microenvironment.

A variety of suitable hydrophilic polymers which may be conjugated to the polycationic polymer scaffolds of the present disclosure are known in the art, including, e.g., synthetic polymers, such as polyethyleneglycol (PEG) and copolymers including PEG, polyvinyl pyrrolidone (PVP), polyvinyl alcohol (PVA), polyacrylic acid (PAA), polyacrylamide, N-(2-hydroxypropyl) methacrylamide (HPMA), divinyl ether-maleic anhydride (DIVEMA), polyoxazoline, polyphosphates, polyphosphazenes; and natural polymers such as, xanthan gum, pectins, chitosan and derivatives thereof, dextran, carrageenan, guar gum, cellulose ethers (e.g., HPMC), hyaluronic acid (HA), albumin, and starch or starch based derivatives.

A polycationic polymer scaffold according to the present disclosure may be modified with one or more of the above hydrophilic polymers or copolymers of two or more of the above hydrophilic polymers.

In some embodiments, a suitable hydrophilic polymer or copolymer of two or more of the above hydrophilic polymers is one which has a weight average molecular weight (Mw) of from about 200 Daltons to about 50 kDa, e.g., from about 400 Daltons to about 50 kDa, from about 600 Daltons to about 50 kDa, from about 800 Daltons to about 50 kDa, from about 1 kDa to about 50 kDa, from about 2 kDa to about 50 kDa, from about 3 kDa to about 50 kDa, from about 4 kDa to about 50 kDa, from about 5 kDa to about 50 kDa, from about 6 kDa to about 50 kDa, from about 7 kDa to about 50 kDa, from about 8 kDa to about 50 kDa, from about 9 kDa to about 50 kDa, from about 10 kDa to about 50 kDa, from about 15 kDa to about 50 kDa, from about 20 kDA to about 50 kDa, from about 25 kDa to about 50 kDa, from about 30 kDa to about 50 kDa, from about 35 kDa to about 50 kDa, from about 40 kDa to about 50 kDa, or from about 45 kDa to about 50 kDa.

In some embodiments, a suitable hydrophilic polymer or copolymer of two or more of the above hydrophilic polymers is one which has a weight average molecular weight (Mw) of from about 2 kDa to about 8 kDa, or from about 4 kDa to about 6 kDa. In some embodiments, a suitable hydrophilic polymer or copolymer of two or more of the above hydrophilic polymers is one which has a weight average molecular weight (Mw) of about 5 kDa. A suitable gel permeation chromatography method may be utilized to determine molecular weight as weight average molecular weight (Mw).

Of particular interest in the present disclosure are polycationic polymer scaffolds covalently modified with a PEG (e.g., to form PPL), e.g., a PEG having a weight average molecular weight (Mw) of from about 200 Daltons to about 50 kDa, e.g., from about 400 Daltons to about 50 kDa, from about 600 Daltons to about 50 kDa, from about 800 Daltons to about 50 kDa, from about 1 kDa to about 50 kDa, from about 2 kDa to about 50 kDa, from about 3 kDa to about 50 kDa, from about 4 kDa to about 50 kDa, from about 5 kDa to about 50 kDa, from about 6 kDa to about 50 kDa, from about 7 kDa to about 50 kDa, from about 8 kDa to about 50 kDa, from about 9 kDa to about 50 kDa, from about 10 kDa to about 50 kDa, from about 15 kDa to about 50 kDa, from about 20 kDA to about 50 kDa, from about 25 kDa to about 50 kDa, from about 30 kDa to about 50 kDa, from about 35 kDa to about 50 kDa, from about 40 kDa to about 50 kDa, or from about 45 kDa to about 50 kDa.

In some embodiments, a suitable PEG has a weight average molecular weight (Mw) of from about 1 kDa to about 10 kDa, e.g., from about 2 kDa to about 8 kDa, or from about 4 kDa to about 6 kDa. In some embodiments, a suitable PEG is one which has a weight average molecular weight (Mw) of about 5 kDa.

In some embodiments, a suitable polycationic polymer-bound hydrophilic polymer is a polycationic polymer-bound PEG (PPL). In some embodiments, a PPL accounts for 14% to 35% of the monomers (i.e., NP polymer scaffold subunits) (e.g., 15% to 35%, 20% to 35%, or 30% to 35%; or 15% to 30%, such as 20% to 25%) which make up a NP according to the present disclosure (i.e., the PPL is present at a molar amount of 14% to 35% relative to the total moles of NP polymer scaffold subunits present in the NP).

Detectable Labels

Detectable labels which may find use in the disclosed nanoparticle compositions generally provide a readily detectable signal which allows for the monitoring and/or detection, e.g., in vitro or in vivo, of the location and/or amount of the polymeric nanparticles. As with the amphiphilic peptide, hydrophilic polymer, and/or target binding moiety, the detectable label may be provided as an individual NP polymer scaffold subunit, with the detectable label covalently bonded to a distinct polycationic polymer scaffold molecule. Alternatively, or in addition, a detectable label may be provided covalently bonded to one or more polycationic polymer scaffold molecules including one or more of an amphiphilic peptide, hydrophilic polymer, and/or target binding moiety covalently bonded thereto.

A variety of suitable detectable labels which may be conjugated to the polycationic polymer scaffolds of the present disclosure are known in the art. Suitable detectable labels include, e.g, radioactive isotopes, fluorophores, chemiluminescers, chromophores, enzymes, enzyme substrates, enzyme cofactors, enzyme inhibitors, dyes, and quantum dots.

In some embodiments, a suitable detectable label is a cyanine dye or a derivative thereof, e.g., Cy3.3™, Cy5.5™, a fluorescein dye or a derivative thereof, a phycoerythrin dye or a derivative thereof, or a rhodamine dye or a derivative thereof. Alexa Fluor® dyes including, e.g., Alexa Fluor® 350, Alexa Fluor® 488, Alexa Fluor® 555, Alexa Fluor® 594, and Alexa Fluor® 647 may also be utilized as detectable labels in the context of the disclosed polymeric nanoparticles. DyLight™ fluors available from Thermo Scientific may also be utilized as detectable labels in the context of the disclosed polymeric nanoparticles.

A particular polycationic polymer scaffold may be modified with one or more of the detectable labels. In addition, where the polycationic polymer scaffold is a peptide, such as a poly-lysine, poly-arginine, or poly-glutamine, a detectable label may be conjugated to the N-terminal, the C-terminal or both the N- and C-terminal of the peptide.

In some embodiments, in addition to, or as an alternative to, directly labeling the polycationic polymer scaffolds of the polymeric nanoparticles, the nucleic acid active agents themselves may be detectably labeled.

In some embodiments, a suitable polycationic polymer-bound label is a polycationic polymer-bound Cy5.5 fluorescent label (CyPL) or polycationic polymer-bound rhodamine (RPL). In some embodiments, a CyPL or an RPL accounts for 1% to 3% of the of the monomers (i.e., NP polymer scaffold subunits) (e.g., 1.5% to 3%, 2% to 3%, or 2.5% to 3%; or 1.5% to 2.5%, such as 2%) which make up a NP according to the present disclosure (i.e., the CyPL or an RPL is present at a molar amount of 1% to 3% relative to the total moles of NP polymer scaffold subunits present in the NP).

Nucleic Acids

As discussed previously herein, the polymeric nanoparticles of the present disclosure facilitate the delivery of nucleic acid active agents within specific cells and/or tissue types. Nucleic acid active agents include nucleic acids as well as precursors, derivatives, prodrugs and analogues thereof, e.g., therapeutic nucleotides, nucleosides and analogues thereof; therapeutic oligonucleotides; and therapeutic polynucleotides. Examples of suitable nucleic acid active agents may include ribozymes, antisense oligodeoxynucleotides, aptamers and interfering RNAs. Examples of suitable nucleoside analogues include, but are not limited to, cytarabine (araCTP), gemcitabine (dFdCTP), and floxuridine (FdUTP).

In some embodiments, a suitable nucleic acid active agent is an interfering RNA, e.g., shRNA, miRNA or siRNA. Suitable interfering RNAs include a sequence complementary to a portion of a gene transcript for a gene product of interest. Of particular interest in connection with the instant disclosure are interfering RNAs which target (via their sequence complementarity to a portion of a gene transcript for a gene product) gene products which have been identified as upregulated (or highly expressed) in cancer cells or otherwise identified as performing a regulatory function with respect to cancer cells.

In some embodiments, suitable interfering RNAs may be associated directly via non-covalent, charge-based interactions with the polycationic polymer scaffolds of the present disclosure to provide the disclosed aggregates and polymeric nanoparticles. In other embodiments, suitable interfering RNAs may be encoded by DNA vectors, e.g., plasmids, which are associated directly via non-covalent, charge-based interactions with the polycationic polymer scaffolds of the present disclosure to provide the disclosed aggregates and polymeric nanoparticles. The interfering RNAs (or precursors thereof) may then be expressed from such vectors following introduction of the nanoparticles into a cell.

In addition, polymeric nanoparticle compositions according to the present disclosure may include one or more different nucleic acid active agents, e.g., a polymeric nanoparticle composition may include a first interfering RNA or a DNA encoding a first interfering RNA bound by ionic-charge interactions to a polycationic polymer scaffold, wherein the interfering RNA includes a sequence complementary to a portion of a gene transcript for a gene product of interest; and a second interfering RNA or a DNA encoding a second interfering RNA bound by ionic-charge interactions to the polycationic polymer scaffold, wherein the second interfering RNA includes a sequence complementary to a portion of a gene transcript for a different gene product of interest. In this manner, polymeric nanoparticles including a plurality of different nucleic acid active agents targeting different gene products of interest may be provided. Such polymeric nanoparticles may include, e.g., 2, 3, 4, 5, or more different nucleic acid active agents targeting different gene products of interest.

Alternatively, or in addition, polymeric nanoparticle compositions according to the present disclosure may include at least two distinct populations of nanoparticles, a first population of nanoparticles, wherein the nanoparticles include a first interfering RNA or a DNA encoding a first interfering RNA bound by ionic-charge interactions to a polycationic polymer scaffold, wherein the interfering RNA includes a sequence complementary to a portion of a gene transcript for a gene product of interest; and a second population of nanoparticles, wherein the nanoparticles include a second interfering RNA or a DNA encoding a second interfering RNA bound by ionic-charge interactions to a polycationic polymer scaffold, wherein the second interfering RNA includes a sequence complementary to a portion of a gene transcript for a different gene product of interest. In this manner, polymeric nanoparticles compositions including a plurality of different polymeric nanoparticle populations, wherein each polymeric nanoparticle population targets a different gene product of interest may be provided. Such polymeric nanoparticles compositions may include, e.g., 2, 3, 4, 5, or more different polymeric nanoparticle populations.

Of particular interest in connection with the present disclosure are interfering RNAs which target gene products which have been identified as upregulated (or highly expressed) in GBM stem cells or otherwise identified as performing a regulatory function with respect to GBM stem cells. Such gene products include, e.g., CD44 (e.g., CD44 V6), c-MET, Sox2, Id-1, Id-3 and NCOA3.

Exemplary DNA sequences encoding shRNAs including a sequence complementary to a portion of a gene transcript for a gene product of interest are provided below in Table 1A. The sense and antisense sequence for each DNA coding sequence for each shRNA is provided. Additional information for the genes/gene products identified in Table 1A is provided below: c-Met: (met proto-oncogene), NCBI Gene ID 4233; Id-1: (inhibitor of DNA binding 1, dominant negative helix-loop-helix protein), NCBI Gene ID 3397; CD-44: (CD44 molecule (Indian blood group)), NCBI Gene ID 960; CD-44 V6: Isoform 6 of CD44, UniProtKB/Swiss-Prot identifier P16070-6); Id-3: (inhibitor of DNA binding 3, dominant negative helix-loop-helix protein), NCBI Gene ID 3399; NCOA3: (nuclear receptor coactivator 3), NCBI Gene ID 8202.

TABLE 1A

| Gene | SEQ | |
|---|---|---|
| Product | ID NO: | Sequence |
| c-Met | 1 | 5′ ACCTCGAACTGGTGTCCCGGATATTCAAGAGATATCCGGGACACCAGTTCTT 3′(sense) |
| | 2 | 5′ CAAAAAGAACTGGTGTCCCGGATATCTCTTGAATATCCGGGACACCAGTTCG 3′ (antisense) |
| c-Met | 3 | 5′ ACCTCGAACAGCGAGCTAAATATATCAAGAGTATATTTAGCTCGCTGTTCTT 3′(sense) |
| | 4 | 5′ CAAAAAGAACAGCGAGCTAAATATACTCTTGATATATTTAGCTCGCTGTTCG 3′ (antisense) |
| c-Met | 5 | 5′ |
| | | GTACCTCGAACTGGTGTCCCGGATATTCAAGAGATATCCGGGACACCAGTTCTTTTTGGAAA |
| | | 3′ (sense) |
| | 6 | 5′ |
| | | AGCTTTTCCAAAAAGAACTGGTGTCCCGGATATCTCTTGAATATCCGGGACACCAGTTCGAG |
| | | 3′ (antisense) |
| c-Met | 7 | 5′ |
| | | GTACCTCGAACAGCGAGCTAAATATATTCAAGAGATATATTTAGCTCGCTGTTCTTTTTGGAA |
| | | A 3′(sense) |
| | 8 | 5′ |
| | | AGCTTTTCCAAAAAGAACAGCGAGCTAAATATATCTCTTGAATATATTTAGCTCGCTGTTCGA |
| | | G 3′ (antisense) |
| c-Met | 9 | 5′ ACCTCGAGCCAGCCTGAATGATGATCAAGAGTCATCATTCAGGCTGGCTCTT 3′(sense) |
| | 10 | 5′ CAAAAAGAGCCAGCCTGAATGATGACTCTTGATCATCATTCAGGCTGGCTCG 3′ (antisense) |
| c-Met | 11 | 5′ ACCTCGTAAGTGCCCGAAGTGTAATCAAGAGTTACACTTCGGGCACTTACTT 3′(sense) |
| | 12 | 5′ CAAAAAGTAAGTGCCCGAAGTGTAACTCTTGATTACACTTCGGGCACTTACG 3′ (antisense) |
| c-Met | 13 | 5′ |
| | | GTACCTCGAGCCAGCCTGAATGATGATCAAGAGTCATCATTCAGGCTGGCTCTTTTTGGAAA |
| | | 3′ (sense) |
| | 14 | 5′ |
| | | AGCTTTTCCAAAAAGAGCCAGCCTGAATGATGACTCTTGATCATCATTCAGGCTGGCTCGAG |
| | | 3′ (antisense) |
| c-Met | 15 | 5′ |
| | | GTACCTCGTAAGTGCCCGAAGTGTAATCAAGAGTTACACTTCGGGCACTTACTTTTTGGAAA |
| | | 3′ (sense) |
| | 16 | 5′ |
| | | AGCTTTTCCAAAAAGTAAGTGCCCGAAGTGTAACTCTTGATTACACTTCGGGCACTTACGAG |
| | | 3′ (antisense) |
| Id-1 | 17 | 5′GTACCTCGCAGGTAAACGTGCTGCTCTATCAAGAGTAGAGCAGCACGTTTACCTGCTTTTT |
| | | GGAAA 3′(sense) |
| | 18 | 5′AGCTTTTCCAAAAAGCAGGTAAACGTGCTGCTCTACTCTTGATAGAGCAGCACGTTTACCT |
| | | GCGAG 3′ (antisense) |
| Id-1 | 19 | 5′ ACCTCGCAGGTAAACGTGCTGCTCTATCAAGAGTAGAGCAGCACGTTTACCTGCTT |
| | | 3′(sense) |
| | 20 | 5′ CAAAAAGCAGGTAAACGTGCTGCTCTACTCTTGATAGAGCAGCACGTTTACCTGCG |
| | | 3′ (antisense) |
| Id-1 | 21 | 5′GTACCTCCCTACTAGTCACCAGAGACTTCTCGAGAAGTCTCTGGTGACTAGTAGGTTTTTG |
| | | GAAA 3′(sense) |
| | 22 | 5′AGCTTTTCCAAAAACCTACTAGTCACCAGAGACTTCTCGAGAAGTCTCTGGTGACTAGTAG |
| | | GGAG 3′ (antisense) |
| Id-1 | 23 | 5′ ACCTCCCTACTAGTCACCAGAGACTTCTCGAGAAGTCTCTGGTGACTAGTAGGTT 3′(sense) |
| | 24 | 5′ CAAAAACCTACTAGTCACCAGAGACTTCTCGAGAAGTCTCTGGTGACTAGTAGGG |
| | | 3′ (antisense) |
| CD-44 | 25 | 5′ ACCTCGCGCAGATCGATTTGAATATCAAGAGTATTCAAATCGATCTGCGCTT 3′(sense) |
| | 26 | 5′ CAAAAAGCGCAGATCGATTTGAATACTCTTGATATTCAAATCGATCTGCGCG 3′ (antisense) |
| CD-44 | 27 | 5′ |
| | | GTACCTCGCGCAGATCGATTTGAATATCAAGAGTATTCAAATCGATCTGCGCTTTTTGGAAA |
| | | 3′(sense) |
| | 28 | 5′ |
| | | AGCTTTTCCAAAAAGCGCAGATCGATTTGAATACTCTTGATATTCAAATCGATCTGCGCGAG |
| | | 3′ (antisense) |
| CD-44 | 29 | 5′ ACCTCGCAACTCCTAGTAGTACAATCAAGAGTTGTACTACTAGGAGTTGCTT 3′(sense) |
| V6 | 30 | 5′ CAAAAAGCAACTCCTAGTAGTACAACTCTTGATTGTACTACTAGGAGTTGCG 3′ (antisense) |
| CD-44 | 31 | 5′ |
| | | GTACCTCGCAACTCCTAGTAGTACAATCAAGAGTTGTACTACTAGGAGTTGCTTTTTGGAAA |
| | | 3′(sense) |
| V6 | 32 | 5′ |
| | | AGCTTTTCCAAAAAGCAACTCCTAGTAGTACAACTCTTGATTGTACTACTAGGAGTTGCGAG |
| | | 3′ (antisense) |
| CD-44 | 33 | 5′ ACCTCTGAGGGATATCGCCAAACATCAAGAGTGTTTGGCGATATCCCTCATT 3′(sense) |
| V6 | 34 | 5′ CAAAAATGAGGGATATCGCCAAACACTCTTGATGTTTGGCGATATCCCTCAG 3′ (antisense) |
| CD-44 | 35 | 5′ |
| | | GTACCTCTGAGGGATATCGCCAAACATCAAGAGTGTTTGGCGATATCCCTCATTTTTGGAAA |
| | | 3′(sense) |
| V6 | 36 | 5′ |
| | | AGCTTTTCCAAAAATGAGGGATATCGCCAAACACTCTTGATGTTTGGCGATATCCCTCAGAG |
| | | 3′ (antisense) |
| CD-44 | 37 | 5′ ACCTCGGCGCAGATCGATTTGAATTCAAGAGATTCAAATCGATCTGCGCCTT 3′(sense) |
| | 38 | 5′ CAAAAAGGCGCAGATCGATTTGAATCTCTTGAATTCAAATCGATCTGCGCCG 3′ (antisense) |
| CD-44 | 39 | 5′ |
| | | GTACCTCGGCGCAGATCGATTTGAATTCAAGAGATTCAAATCGATCTGCGCCTTTTTGGAAA |
| | | 3′(sense) |
| | 40 | 5′ |
| | | AGCTTTTCCAAAAAGGCGCAGATCGATTTGAATCTCTTGAATTCAAATCGATCTGCGCCGAG |
| | | 3′ (antisense) |
| Id-3 | 41 | 5′ACCTCACTCAGCTTAGCCAGGTGGAAATCCTACATCAAG4GTGTAGG4TTTCCACCTGGCT |
| | | AAGCTGAGTTT 3′(sense) |
| | 42 | 5′CAAAAAACTCAGCTTAGCCAGGTGGAAATCCTACACTCTTGATGTAGGATTTCCACCTGGC |
| | | TAAGCTGAGTG 3′(antisense) |
| Id-3 | 43 | 5′GTACCTCACTCAGCTTAGCCAGGTGGAAATCCTACATCAAGAGTGTAGGATTTCCACCTGG |
| | | CTAAGCTGAGTTTTTTGGAAA 3′(sense) |
| | 44 | 5′AGCTTTTCCAAAAAACTCAGCTTAGCCAGGTGGAAATCCTACACTCTTGATGTAGGATTTCC |
| | | ACCTGGCTAAGCTGAGTGAG 3′(antisense) |
| Id-3 | 45 | 5′ACCTCATCGACTACATTCTCGACCTGCAGGTAGTTCAAGAGACTACCTGCAGGTCGAGAAT |
| | | GTAGTCGATTT 3′(sense) |
| | 46 | 5′CAAAAAATCGACTACATTCTCGACCTGCAGGTAGTCTCTTGAACTACCTGCAGGTCGAGAA |
| | | TGTAGTCGATG 3′(antisense) |
| Id-3 | 47 | 5′GTACCTCATCGACTACATTCTCGACCTGCAGGTAGTTCAAGAGACTACCTGCAGGTCGAGA |
| | | ATGTAGTCGATTTTTTGGAAA 3′(sense) |
| | 48 | 5′AGCTTTTCCAAAAAATCGACTACATTCTCGACCTGCAGGTAGTCTCTTGAACTACCTGCAGG |
| | | TCGAGAATGTAGTCGATGAG 3′(antisense) |
| Id-3 | 49 | 5′ACCTCACCTTCCCATCCAGACAGCCGAGCTCACTTCAAGAGAGTGAGCTCGGCTGTCTGGA |
| | | TGGGAAGGTTT 3′(sense) |
| | 50 | 5′CAAAAAACCTTCCCATCCAGACAGCCGAGCTCACTCTCTTGAAGTGAGCTCGGCTGTCTGG |
| | | ATGGGAAGGTG 3′ (antisense) |
| Id-3 | 51 | 5′GTACCTCACCTTCCCATCCAGACAGCCGAGCTCACTTCAAG4G4GTG4GCTCGGCTGTCTG |
| | | GATGGGAAGGTTTTTTGGAAA 3′(sense) |
| | 52 | 5′AGCTTTTCCAAAAAACCTTCCCATCCAGACAGCCGAGCTCACTCTCTTGAAGTGAGCTCGG |
| | | CTGTCTGGATGGGAAGGTGAG 3′(antisense) |
| Id-3 | 53 | 5′ACCTCCCGGAACTTGTCATCTCCAACGACAAAAGTCAAGAGCTTTTGTCGTTGGAGATGAC |
| | | AAGTTCCGGTT 3′(sense) |
| | 54 | 5′CAAAAACCGGAACTTGTCATCTCCAACGACAAAAGCTCTTGACTTTTGTCGTTGGAGATGA |
| | | CAAGTTCCGGG 3′(antisense) |
| Id-3 | 55 | 5′GTACCTCCCGGAACTTGTCATCTCCAACGACAAAAGTCAAGAGCTTTTGTCGTTGGAGATG |
| | | ACAAGTTCCGGTTTTTGGAAA 3′(sense) |
| | 56 | 5′AGCTTTTCCAAAAACCGGAACTTGTCATCTCCAACGACAAAAGCTCTTGACTTTTGTCGTTG |
| | | GAGATGACAAGTTCCGGGAG 3′ (antisense) |
| NCOA3 | 57 | 5′ ACCTCGCAGTCTATTCGTCCTCCATATCAAGAGTATGGAGGACGAATAGACTGCTT 3′(sense) |
| | 58 | 5′ CAAAAAGCAGTCTATTCGTCCTCCATACTCTTGATATGGAGGACGAATAGACTGCG 3′ |
| | | (antisense) |
| NCOA3 | 59 | 5′GTACCTCGCAGTCTATTCGTCCTCCATATCAAGAGTATGGAGGACGAATAGACTGCTTTTT |
| | | GGAAA 3′(sense) |
| | 60 | 5′AGCTTTTCCAAAAAGCAGTCTATTCGTCCTCCATACTCTTGATATGGAGGACGAATAGACTG |
| | | CGAG 3′ (antisense) |

Accordingly, in some embodiments, suitable interfering RNAs which may be delivered using the disclosed polymeric nanoparticles (e.g. in the form of plasmid DNA encoding same) are shRNAs encoded by DNA plasmids including a DNA sequence as identified in Table 1A or a DNA sequence which is substantially homologous to a DNA sequence identified in Table 1A, e.g., a DNA sequence having at least about 80% sequence identity, e.g., at least about 85% sequence identity, at least about 90% sequence identity, at least about 95% sequence identity, at least about 98% sequence identity, or at least about 99% sequence identity with one of the DNA sequences identified in Table 1A. In some embodiments, a suitable shRNA is one which is encoded by a DNA plasmid including a DNA sequence having about 80% to about 99% sequence identity, e.g., from about 85% to about 99%, from about 90% to about 99%, from about 95% to about 99%, or from about 98% to about 99% sequence identity with one of the sequences set forth in Table 1A.

Exemplary siRNA nucleic acid sequences are provided below in Table 1B.

TABLE 1B

| | Sense (5′-3′) | Antisense (5′-3′) |
|---|---|---|
| BPTF | rCrArUrArArUrArUrCrGrUrCrUrUrUrGrUr | rArGrArGrUrArCrArArArGrArCrGrArUrArUrUr |
| siRNA1 | ArCrUrCrUraaa | ArUrGraaa |
| CD44 siRNA | rGrCrGrCrArGrArUrCrGrArUrUrUrGrArAr | rUrArUrUrCrArArArUrCrGrArUrCrUrGrCrGrCc |
| | UrAtt | a |
| Sox2 | rUrGrGrUrCrArUrGrGrArGrUrUrCrUrArCr | rCrArGrUrArCrArArCrUrCrCrArUrGrArCrCrAc |
| siRNA1 | UrGca | g |
| Sox2 siRNA2 | rUrUrCrArUrGrUrArGrGrUrCrUrGrCrGrAr | rGrCrUrCrGrCrArGrArCrCrTrArCrArTrGrArAc |
| | GrCtg | g |
| Sox2 siRNA3 | rUrArCrUrUrArUrCrCrUrUrCrUrUrCrArUr | rUrCrArUrGrArArGrArArGrGrArUrArArGrUrAc |
| | GrAgc | a |
| Sox2 siRNA4 | rArArCrCrCrArUrGrGrArGrCrCrArArGrAr | rGrCrUrCrUrUrGrGrCrUrCrCrArUrGrGrGrUrUc |
| | GrCca | g |

Exemplary miRNA nucleic acid sequences are provided below in Table 1C.

TABLE 1C

| | |
|---|---|
| hsa-miR-34a- | UGGCAGUGUCUUAGCUGGUUGU, which could also be used as the |
| 5p | following stem-loop sequence: hsa-mir-34a MI0000268 |
| MI00002268 | GGCCAGCUGUGAGUGUUUCUUUGGCAGUGUCUUAGCUGGUUGUUG |
| mature | UGAGCAAUAGUAAGGAAGCAAUCAGCAAGUAUACUGCCCUAGAAG |
| sequence | UGCUGCACGUUGUGGGGCCC |
| hsa-miR-128- | GGGGGCCGAUACACUGUACGAGA, which could also be used as the |
| 2-5p | following stem-loop sequence: hsa-mir-128-2 MI0000727 |
| MI0000727 | UGUGCAGUGGGAAGGGGGGCCGAUACACUGUACGAGAGUGAGUAG |
| mature | CAGGUCUCACAGUGAACCGGUCUCUUUCCCUACUGUGUC |
| sequence | |

Accordingly, in some embodiments, suitable siRNAs and miRNAs which may be delivered using the disclosed polymeric nanoparticles include a nucleic acid sequence as set forth in Table 1B and 1C or a nucleic acid sequence which is substantially homologous to a nucleic acid sequence identified in Tables 1B and 1C, e.g., a nucleic acid sequence having at least about 80% sequence identity, e.g., at least about 85% sequence identity, at least about 90% sequence identity, at least about 95% sequence identity, at least about 98% sequence identity, or at least about 99% sequence identity with one of the nucleic acid sequences identified in Tables 1B or 1C. In some embodiments, a suitable siRNA or miRNA is one which includes a nucleic acid sequence having about 80% to about 99% sequence identity, e.g., from about 85% to about 99%, from about 90% to about 99%, from about 95% to about 99%, or from about 98% to about 99% sequence identity with one of the sequences set forth in Tables 1B or 1C.

In addition, a variety of methods and techniques are known in the art for selecting a particular mRNA target sequence during interfering RNA design. See, e.g., the publicly available siRNA design tool provided by the Whitehead Institute of Biomedical Research at MIT. This tool can be located on the internet on the website located by placing http:// directly preceding jura.wi.mit.edu/bioc/siRNAext/.

In some embodiments, nanoparticles according to the present disclosure can be used to deliver one or more nucleic acids encoding one or more therapeutic proteins or peptides of interest, e.g., plasmids containing one or more nucleic acids encoding one or more therapeutic proteins or peptides of interest. Such nanoparticles can be used to selectively express therapeutic proteins or peptides, e.g., in a desired tissue, cell or location in the body.

Methods of Making

Exemplary methods of making the polymeric nanoparticles of the present disclosure are provided below and in the following Examples. Generally, such methods include combining nucleic acid active agents of interest with covalently-modified polycationic polymer scaffolds as described herein, wherein the combining results in condensation of the nucleic acids and formation of polymeric nanoparticles. Such combining generally takes place at a molar ratio of nucleic acid to polycationic polymer scaffold of from about 1:100 to about 1:1000, e.g., from about 1:100 to about 1:900, from about 1:100 to about 1:800, from about 1:100 to about 1:700, from about 1:100 to about 1:600, from about 1:100 to about 1:500, from about 1:100 to about 1:400, from about 1:100 to about 1:300, or from about 1:100 to about 1:200.

Polymeric nanoparticles of the present disclosure may also be prepared by combining nucleic acid active agents of interest with covalently-modified polycationic polymer scaffolds as described herein with reference to the ratio of nitrogen atoms (N) and phosphate groups (P) on the polycationic polymer scaffolds and the nucleic acid active agents respectively. A suitable N:P ratio for the combination of the polycationic polymer scaffolds and the nucleic acid active agents respectively is from about 1:1 to about 1:100, e.g., from about 1:10 to about 1:20, from about 1:20 to about 1:30, from about 1:30 to about 1:40, from about 1:40 to about 1:50, from about 1:50 to about 1:60, from about 1:60 to about 1:70; from about 1:70 to about 1:80, from about 1:80 to about 1:90, or from about 1:90 to about 1:100.

In some embodiments, a suitable N:P ratio for the combination of the polycationic polymer scaffolds and the nucleic acid active agents respectively is from about 1:5 to 1:32, e.g., from about 1:6 to about 1:32, from about 1:7 to about 1:32, from about 1:8 to about 1:32, from about 1:9 to about 1:32, from about 1:10 to about 1:32, from about 1:11 to about 1:32, from about 1:12 to about 1:32, from about 1:13 to about 1:32, from about 1:14 to about 1:32, from about 1:15 to about 1:32, from about 1:16 to about 1:32, from about 1:17 to about 1:32, from about 1:18 to about 1:32, from about 1:19 to about 1:32, from about 1:20 to about 1:32, from about 1:21 to about 1:32, from about 1:22 to about 1:32, from about 1:23 to about 1:32, from about 1:24 to about 1:32, from about 1:25 to about 1:32, from about 1:26 to about 1:32, from about 1:27 to about 1:32, from about 1:28 to about 1:32, from about 1:29 to about 1:32, from about 1:30 to about 1:32, or from about 1:31 to about 1:32. In some embodiments of particular interest an N:P ratio of 1:32 is utilized.

In some embodiments, a suitable N:P ratio for the combination of the polycationic polymer scaffolds and the nucleic acid active agents respectively is from about 1:32 to 1:67 (i.e., 1 phosphate on the nucleic acid backbone to 32 to 67 nitrogen atoms on the NP polymer scaffold subunits; thus each phosphate on the nucleic acid backbone is ionically captured with 32 to 67 nitrogen atoms on the NP polymer scaffold subunits), e.g., 1:34 to 1:67, 1:36 to 1:67, 1:38 to 1:67, 1:40 to 1:67, 1:42 to 1:67, 1:44 to 1:67, 1:46 to 1:67, 1:48 to 1:67, 1:50 to 1:67, 1:52 to 1:67, 1:54 to 1:67, 1:56 to 1:67, 1:58 to 1:67, 1:60 to 1:67, 1:62 to 1:67, 1:64 to 1:67, or 1:66 to 1:67.

For embodiments, such as those described herein, wherein the covalently-modified polycationic polymer scaffolds include an amphiphilic peptide as described herein, the method may include a step of covalently bonding a polycationic polymer scaffold to an amphiphilic peptide at a molar ratio of from about 1:1 to about 1:10, e.g., about 1:1 to about 1:9, about 1:1 to about 1:8, about 1:1 to about 1:7, about 1:1 to about 1:6, about 1:1 to about 1:5, about 1:1 to about 1:4, about 1:1 to about 1:3, or about 1:1 to about 1:2.

For embodiments, such as those described herein, wherein the covalently-modified polycationic polymer scaffolds include a target binding moiety, e.g., a first or second target binding moiety as described herein, the method may include a step of covalently bonding a polycationic polymer scaffold to a target binding moiety at a molar ratio of about 1:1 to about 1:10, e.g., about 1:1 to about 1:9, about 1:1 to about 1:8, about 1:1 to about 1:7, about 1:1 to about 1:6, about 1:1 to about 1:5, about 1:1 to about 1:4, about 1:1 to about 1:3, or about 1:1 to about 1:2, to provide a covalently-modified polycationic polymer scaffold.

For embodiments, such as those described herein, wherein the covalently-modified polycationic polymer scaffolds include a BBB transport moiety, the method may include a step of covalently bonding a polycationic polymer scaffold to a BBB transport moiety at a molar ratio of from about 1:1 to about 1:5, e.g., from about 1:1 to about 1:4, from about 1:1 to about 1:3, or from about 1:1 to about 1:2.

For embodiments, such as those described herein, wherein the covalently-modified polycationic polymer scaffolds include a hydrophilic polymer as described herein, the method may include a step of bonding the hydrophilic polymer to a polycationic polymer scaffold at a molar ratio of about 6:1 to about 2:1, e.g., about 5:1 to about 3:1, or about 4:1, to provide a covalently-modified polycationic polymer scaffold.

As discussed herein, preparation of the disclosed polymeric nanoparticles involves the combination of covalently-modified polycationic polymer scaffolds with nucleic acid active agents to form aggregates. A variety of methods are known in the art which may be utilized to covalently modify the polycationic polymer scaffolds with one or more of the amphiphilic peptides, target binding moieties, BBB transport moieties and detectable labels described herein.

Suitable methods, in addition to those described in the Examples, may include, but are not limited to, carbodiimide coupling reactions, copper-catalyzed azide/alkyne [3+2] cycloaddition “Click Chemistry,” azide/DIFO (Difluorinated Cyclooctyne) or copper-free Click Chemistry, azide/phosphine “Staudinger Reaction,” azide/triarylphosphine “Modified Staudinger Reaction,” and olefin metathesis reactions.

The polycationic polymer scaffolds described herein may be characterized as having a first terminal end, a second terminal end, and an intermediate region extending between the first terminal end and the second terminal end. Where the polycationic polymer scaffold is a peptide, the first terminal end may be an N- or C-terminus and the second terminal end may be a C- or N-terminus accordingly. The polycationic polymer scaffolds may be covalently modified with one or more of the amphiphilic peptides, target binding moieties, BBB transport moieties and detectable labels described herein such that one of these components is positioned at the first terminal end, the second terminal end, or as a pendant modification to the intermediate region of the polycationic polymer scaffold.

By way of example, FIG. 1A provides a schematic of an embodiment, wherein a hydrophilic polymer is covalently attached as a pendant moiety to the intermediate region of the polycationic polymer scaffold. Similarly, FIG. 1A depicts schematically an embodiment in which an amphiphilic peptide/target binding moiety is covalently attached as a pendant moiety to the intermediate region of the polycationic polymer scaffold.

By comparison, FIG. 1D provides a schematic of an embodiment in which a BBB transport moiety is covalently attached to, e.g., a first terminal end, and a detectable label is covalently attached to, e.g., a second terminal end. As in FIG. 1A, in FIG. 1D, the amphiphilic peptide/target binding moiety and the hydrophilic polymer are each covalently attached as pendant moieties to the intermediate region of the polycationic polymer scaffold. A variety of additional configurations of the components discussed herein will be readily recognizable by one of ordinary skill in the art upon reading the present disclosure and such configurations are considered a part of the present disclosure. As long as the covalently conjugated moieties are geometrically non-hindering and configured to allow for sufficient exposure of positively charged moieties for interaction with the negatively charged nucleic acids, they may be bound at any suitable position on the polycationic polymer scaffold.

Pharmaceutical Formulations and Dosage Forms Including Polymeric Nanoparticles

One skilled in the art will appreciate that a variety of suitable methods of administering a polymeric nanoparticle composition to a subject or host, e.g., patient, in need thereof, are available, and, although more than one route can be used to administer a particular composition, a particular route can provide a more immediate and more effective reaction than another route. Pharmaceutically acceptable excipients are also well known to those who are skilled in the art, and are readily available. The choice of excipient will be determined in part by the particular compound, as well as by the particular method used to administer the composition. Accordingly, there are a wide variety of suitable formulations of the polymeric nanoparticle compositions. The following methods and excipients are merely exemplary and are in no way limiting.

Formulations suitable for oral administration can consist of (a) liquid solutions, such as an effective amount of the polymeric nanoparticle composition dissolved in diluents, such as water or saline; (b) capsules, sachets or tablets, each containing a predetermined amount of the polymeric nanoparticle composition, as solids or granules; (c) suspensions in an appropriate liquid; (d) suitable emulsions and (e) hydrogels. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles including the active ingredient in an inert base, such as gelatin and glycerin, or sucrose and acacia, emulsions, gels, and the like containing, in addition to the active ingredient, such excipients as are known in the art.

Polymeric nanoparticle formulations can be made into aerosol formulations to be administered via inhalation. These aerosol formulations can be placed into pressurized acceptable propellants, such as dichlorodifluoromethane, propane, nitrogen, and the like. They may also be formulated as pharmaceuticals for non-pressured preparations such as for use in a nebulizer or an atomizer.

Formulations suitable for parenteral administration, e.g., in the context of treatment of cancer, e.g., GBM, may include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Formulations suitable for topical administration may be presented as creams, gels, pastes, patches, sprays or foams.

Suppository formulations are also provided by mixing with a variety of bases such as emulsifying bases or water-soluble bases. Formulations suitable for vaginal administration may be presented as pessaries, tampons, creams, gels, pastes, foams

Unit dosage forms for oral or rectal administration such as syrups, elixirs, and suspensions may be provided wherein each dosage unit, for example, teaspoonful, tablespoonful, tablet or suppository, contains a predetermined amount of the composition. Similarly, unit dosage forms for injection or intravenous administration may comprise the polymeric nanoparticles in a formulation as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of polymeric nanoparticle composition calculated in an amount sufficient to produce the desired effect in association with a pharmaceutically acceptable diluent, carrier or vehicle. The specifications for the novel unit dosage forms of the polymeric nanoparticle compositions may depend on the particular nucleic acid active agent employed and the effect to be achieved, and the pharmacodynamics associated with each compound in the host.

Those of skill in the art will readily appreciate that dose levels can vary as a function of the specific nucleic acid active agent or polymeric nanoparticle composition, the nature of the delivery vehicle, and the like. Suitable dosages for a given nucleic acid active agent or polymeric nanoparticle composition are readily determinable by those of skill in the art by a variety of means. In some embodiments, a suitable dose may include from about 1×10⁵ to about 1×10¹² polymeric nanoparticles per Kg body weight, delivered, e.g., via injection. For example, in some embodiments, a suitable dose may include from about 1×10⁶ to about 1×10¹¹, from about to 1×10⁷ to 1×10¹⁰, or from about 1×10⁸ to about 1×10⁹ polymeric nanoparticles per Kg body weight. In some embodiments, a suitable dose may include from about 1×10⁵ to about 1×10⁶, from about 1×10⁶ to about 1×10⁷, from about 1×10⁷ to about 1×10⁸, from about 1×10⁸ to about 1×10⁹, from about 1×10⁹ to about 1×10¹⁰, or from about 1×10¹⁰ to about 1×10¹¹ polymeric nanoparticles per Kg body weight.

Optionally, the pharmaceutical composition may contain other pharmaceutically acceptable components, such a buffers, surfactants, antioxidants, viscosity modifying agents, preservatives and the like. Each of these components is well-known in the art. See, e.g., U.S. Pat. No. 5,985,310, the disclosure of which is incorporated herein by reference.

Other components suitable for use in polymeric nanoparticle formulations can be found in Remington's Pharmaceutical Sciences, Mack Pub. Co., 18th edition (June 1995).

The polymeric nanoparticles of the present disclosure may be present in the disclosed polymeric nanoparticle compositions in any suitable concentration. Suitable concentrations may vary depending on the potency or concentration of the nucleic acid active agent, active agent half-life, etc.

Polymeric Nanoparticle Compositions as Medical Device Components

In some embodiments, one or more of the polymeric nanoparticle compositions of the present disclosure may be incorporated into a medical device known in the art, for example, drug eluting stents, catheters, fabrics, cements, bandages (liquid or solid), biodegradable polymer depots and the like. In such embodiments, the polymeric nanoparticle composition may, for example, be applied as a coating or deposited within the medical device. In some embodiments, the medical device is an implantable or partially implantable medical device.

Methods of Treatment

The terms “an effective amount” (or, in the context of a therapy, a “pharmaceutically effective amount”) of a polymeric nanoparticle composition generally refers to an amount of the polymeric nanoparticle composition, effective to accomplish the desired therapeutic effect, e.g., in the case of a polymeric nanoparticle composition including interfering RNA, an amount effective to reduce expression of the targeted mRNA by an amount effective to produce a desired therapeutic effect.

In the case of the treatment of cancer, a desired therapeutic effect may be a reduction in tumor size, a reduction in the proliferation of cancer cells, and/or a reduction in the likelihood of recurrence.

Effective amounts of polymeric nanoparticle compositions, suitable delivery vehicles, and protocols can be determined by conventional means. For example, in the context of therapy a medical practitioner can commence treatment with a low dose of one or more polymeric nanoparticle compositions in a subject or patient in need thereof, and then increase the dosage, or systematically vary the dosage regimen, monitor the effects thereof on the patient or subject, and adjust the dosage or treatment regimen to maximize the desired therapeutic effect. Further discussion of optimization of dosage and treatment regimens can be found in Benet et al., in Goodman & Gilman's The Pharmacological Basis of Therapeutics, Ninth Edition, Hardman et al., Eds., McGraw-Hill, New York, (1996), Chapter 1, pp. 3-27, and L. A. Bauer, in Pharmacotherapy, A Pathophysiologic Approach, Fourth Edition, DiPiro et al., Eds., Appleton & Lange, Stamford, Conn., (1999), Chapter 3, pp. 21-43, and the references cited therein, to which the reader is referred.

The dosage levels and mode of administration will be dependent on a variety of factors such as the specific polymeric nanoparticles used, the nucleic acid active agent, the context of use (e.g., the patient to be treated), and the like. Optimization of modes of administration, dosage levels, and adjustment of protocols, including monitoring systems to assess effectiveness are routine matters well within ordinary skill.

In some embodiments, a suitable dose may include from about 1×10⁵ to about 1×10¹² polymeric nanoparticles per Kg body weight, delivered, e.g., via injection. For example, in some embodiments, a suitable dose may include from about 1×10⁶ to about 1×10¹¹, from about to 1×10⁷ to 1×10¹⁰, or from about 1×10⁸ to about 1×10⁹ polymeric nanoparticles per Kg body weight. In some embodiments, a suitable dose may include from about 1×10⁵ to about 1×10⁶, from about 1×10⁶ to about 1×10⁷, from about 1×10⁷ to about 1×10⁸, from about 1×10⁸ to about 1×10⁹, from about 1×10⁹ to about 1×10¹⁰, or from about 1×10¹⁰ to about 1×10¹¹ polymeric nanoparticles per Kg body weight.

In one embodiment, the present disclosure provides a method of treating a subject having, suspected of having or susceptible to a disorder resulting at least in part from expression of an mRNA, including administering to the subject a pharmaceutically effective amount of a composition including a polymeric nanoparticle composition as described herein, wherein the polymeric nanoparticle composition includes as the nucleic acid active agent an interfering RNA or a DNA encoding an interfering RNA bound by ionic-charge interactions to the polycationic polymer scaffold, wherein the interfering RNA comprises a sequence complementary to a portion of the mRNA, and whereby expression of the mRNA is reduced relative to expression of the mRNA in the absence of the contacting.

In embodiments of particular interest, the interfering RNA comprises a sequence complementary to a portion of a gene transcript for CD-44, CD-44 V6, Sox2, Id-1, Id-3, c-Met, or NCOA3.

As some miRNAs function to increase the expression of specific target genes, in some embodiments, the present disclosure provides methods of increasing expression of a target protein in a cell, wherein the method includes contacting the cell with a polymeric nanoparticle as described herein which includes a miRNA or a DNA encoding a miRNA.

In some embodiments, the disclosed NPs can be used to overexpress one or more miRNAs in one or more cells or tissues. For example, NPs can be used to deliver mir-34a or mir-128, e.g., as described in Example 11.

In some embodiments, methods of treating Glioblastoma Multiforme (GBM) in a subject having, suspected of having or susceptible to GBM are provided, wherein the methods include administering a therapeutically effective amount of a formulation including a plurality of polymeric nanoparticles as described herein to the subject, wherein the polymeric nanoparticles include an interfering RNA or a DNA encoding an interfering RNA bound by ionic-charge interactions to the polycationic polymer scaffold, wherein the interfering RNA comprises a sequence complementary to a portion of a gene transcript for CD-44, CD-44 V6, Sox2, Id-1, Id-3, c-Met, or NCOA3.

In particular embodiments of interest, the subject is identified as one who has or has had a CD-44 V6 expressing tumor and the interfering RNA comprises a sequence complementary to a portion of a gene transcript for CD-44 V6.

Knockdown of one cancer cell marker, e.g., a GBM stem cell marker, with interfering RNA may result in upregulation of other markers as a compensatory mechanism in tumor progression. Accordingly, in some embodiments, a method of treatment according to the present disclosure includes the administration of a polymeric nanoparticle composition including a first interfering RNA or a DNA encoding a first interfering RNA bound by ionic-charge interactions to a polycationic polymer scaffold, wherein the interfering RNA includes a sequence complementary to a portion of a gene transcript for one of CD-44, CD-44 V6, Sox2, Id-1, Id-3, c-Met, and NCOA3; and a second interfering RNA or a DNA encoding a second interfering RNA bound by ionic-charge interactions to the polycationic polymer scaffold, wherein the second iRNA includes a sequence complementary to a portion of a gene transcript for a different one of CD-44, CD-44 V6, Sox2, Id-1, Id-3, c-Met, and NCOA3.

Alternatively, or in addition, the method may include the administration of a polymeric nanoparticle composition including at least two distinct populations of nanoparticles, a first population of nanoparticles, wherein the nanoparticles include a first interfering RNA or a DNA encoding a first interfering RNA bound by ionic-charge interactions to a polycationic polymer scaffold, wherein the interfering RNA includes a sequence complementary to a portion of a gene transcript for one of CD-44, CD-44 V6, Sox2, Id-1, Id-3, c-Met, and NCOA3; and a second population of nanoparticles, wherein the nanoparticles include a second interfering RNA or a DNA encoding a second interfering RNA bound by ionic-charge interactions to a polycationic polymer scaffold, wherein the second interfering RNA includes a sequence complementary to a portion of a gene transcript for a different one of CD-44, CD-44 V6, Sox2, Id-1, Id-3, c-Met, and NCOA3.

Combination Treatments

In some embodiments, the polymeric nanoparticle compositions according to the present disclosure may be administered as part of a combination therapy which includes the administration of one or more known anticancer agents. For example, in some embodiments, for the treatment of GBM, a polymeric nanoparticle composition according to the present disclosure may be administered as part of a combination therapy with temozolomide (TMZ).

In addition, or alternatively, the polymeric nanoparticle compositions according to the present disclosure may be administered as part of a combination therapy which includes a surgical resection procedure, radiation therapy, and/or the administration of a second chemotherapeutic.

In some embodiments, polymeric nanoparticle compositions according to the present disclosure may be administered prior to or subsequent to one or more of a surgical resection procedure, radiation therapy, and/or the administration of a second chemotherapeutic.

In-Vitro Use

In addition to treatment methods and other in-vivo uses, the polymeric nanoparticle compositions disclosed herein may also be used in the context of in-vitro experimentation. For example, the polymeric nanoparticles disclosed herein may be used to deliver any of a wide variety of nucleic acid active agents as discussed herein, as well as potential nucleic acid active agents, into viable cells in-vitro to determine the potential therapeutic effect, toxicity, etc. of the nucleic acid active agent or potential nucleic acid active agent. For this reason, the polymeric nanoparticle compositions of the present disclosure may be useful in the context of drug testing and/or screening.

In some embodiments, polymeric nanoparticle compositions as described herein may be used in in-vitro gene silencing experiments, e.g., by introducing a polymeric nanoparticle composition according to the present disclosure, wherein the polymeric nanoparticle composition includes an interfering RNA or a DNA encoding an interfering RNA, wherein the interfering RNA includes a sequence complementary to a portion of a gene transcript for a target gene and monitoring the effect on expression of the target gene.

Additional in-vitro uses may include the use of polymeric nanoparticles as disclosed herein, wherein the polymeric nanoparticles include one or more detectable labels (e.g., fluorescent labels or radioactive labels) in order to label viable cells in-vitro.

Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-261 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.
1. A polymeric nanoparticle including an aggregate of nucleic acids and polycationic polymer scaffolds, wherein the aggregate includes:
   a polycationic polymer scaffold;
   an amphiphilic peptide covalently bound to the polycationic polymer scaffold;
   a hydrophilic polymer covalently bound to the polycationic polymer scaffold; and
   a nucleic acid bound by ionic-charge interactions to the polycationic polymer scaffold.
2. The polymeric nanoparticle of 1, wherein the amphiphilic peptide includes a first target binding moiety.
3. The polymeric nanoparticle of 4, wherein the amphiphilic peptide includes chlorotoxin (ClTx).
4. The polymeric nanoparticle of any one of 1-3, including a second target binding moiety covalently bound to the polycationic polymer scaffold.
5. The polymeric nanoparticle of 4, wherein the second target binding moiety includes a first blood brain barrier (BBB) transport moiety.
6. The polymeric nanoparticle of any one of 1-5, including a second BBB transport moiety covalently bound to the polycationic polymer scaffold.
7. The polymeric nanoparticle of 5 or 6, wherein the first and/or second BBB transport moiety includes a peptide.
8. The polymeric nanoparticle of any one of 5-7, wherein the first and/or second BBB transport moiety includes a transferrin receptor ligand.
9. The polymeric nanoparticle of any one of 5-8, wherein the molar ratio of the polycationic polymer scaffold to the first and/or second BBB transport moiety is about 1:1 to about 1:2.
10. The polymeric nanoparticle of any one of 1-9, including a detectable label covalently bound to the polycationic polymer scaffold.
11. The polymeric nanoparticle of any one of 1-10, wherein the detectable label includes a fluorescent label.
12. The polymeric nanoparticle of any one of 1-11, wherein the polycationic polymer scaffold includes one or more of poly-lysine, poly-arginine, poly-glutamine, poly-amine, poly(diallydimethylammonium chloride) (pDADMAC), and chitosan.
13. The polymeric nanoparticle of any one of 1-12, wherein the second target binding moiety includes a polypeptide.
14. The polymeric nanoparticle of any one of 1-13, wherein the second target binding moiety includes an antibody.
15. The polymeric nanoparticle of any one of 1-12, wherein the second target binding moiety includes an aptamer.
16. The polymeric nanoparticle of any one of 1-15, wherein the molar ratio of the polycationic polymer scaffold to the first target binding moiety is about 1:1 to about 1:5.
17. The polymeric nanoparticle of any one of 1-16, wherein the hydrophilic polymer has a Mw of from about 2 kDa to about 10 kDa.
18. The polymeric nanoparticle of 17, wherein the hydrophilic polymer has a Mw of about 5 kDa.
19. The polymeric nanoparticle of any one of 1-18, wherein the hydrophilic polymer includes a polyethyleneglycol (PEG).
20. The polymeric nanoparticle of any one of 1-19, wherein the nucleic acid includes DNA.
21. The polymeric nanoparticle of 20, wherein the DNA encodes an interfering RNA.
22. The polymeric nanoparticle of 21, wherein the interfering RNA includes a short-hairpin RNA (shRNA).
23. The polymeric nanoparticle of 21 or 22, wherein the interfering RNA includes a sequence complementary to a portion of a gene transcript for CD-44, CD-44 V6, Sox2, Id-1, Id-3, c-Met, or NCOA3.
24. The polymeric nanoparticle of any one of 1-19, wherein the nucleic acid includes RNA.
25. The polymeric nanoparticle of 24, wherein the RNA includes interfering RNA.
26. The polymeric nanoparticle of 25, wherein the interfering RNA includes a small interfering RNA (siRNA).
27. The polymeric nanoparticle of 25, wherein the interfering RNA includes a shRNA.
28. The polymeric nanoparticle of 25, wherein the interfering RNA includes a micro-RNA (miRNA).
29. The polymeric nanoparticle of any one of 25-28, wherein the interfering RNA includes a sequence complementary to a portion of a gene transcript for CD-44, CD-44 V6, Sox2, Id-1, Id-3, c-Met, or NCOA3.
30. The polymeric nanoparticle of any one of 1-29, wherein the ratio of nucleic acids to polycationic polymer scaffolds in the nanoparticle is from about 1:100 to about 1:1000.
31. The polymeric nanoparticle of any one of 1-29, wherein the ratio of nitrogen (N) to phosphate (P) in the nanoparticle is from about 1:1 to about 1:100.
32. A method of reducing the expression of a target protein in a cell, the method including
   contacting the cell with a polymeric nanoparticle, the polymeric nanoparticle including an aggregate of nucleic acids and polycationic polymer scaffolds, wherein the aggregate includes:
   a polycationic polymer scaffold;
   an amphiphilic peptide covalently bound to the polycationic polymer scaffold;
   a hydrophilic polymer covalently bound to the polycationic polymer scaffold; and
   an interfering RNA or a DNA encoding an interfering RNA bound by ionic-charge interactions to the polycationic polymer scaffold, wherein the interfering RNA includes a sequence complementary to a portion of a gene transcript for the target protein, and whereby expression of the target protein is reduced relative to expression of the target protein in the absence of the contacting.
33. The method of 32, wherein the contacting is in vitro.
34. The method of 32, wherein the contacting is in vivo.
35. The method of 34, wherein the cell is in a mammal.
36. The method of 35, wherein the cell is in a human.
37. The method of any one of 32-36, wherein the cell is a cancer cell.
38. The method of 37, wherein the cancer cell is a glioma cell.
39. The method of any one of 32-38, wherein the amphiphilic peptide includes a first target binding moiety.
40. The method of 39, wherein the amphiphilic peptide includes chlorotoxin (ClTx).
41. The method of any one of 32-40, wherein the aggregate includes a second target binding moiety covalently bound to the polycationic polymer scaffold.
42. The method of 41, wherein the second target binding moiety includes a first blood brain barrier (BBB) transport moiety.
43. The method of any one of 32-42, wherein the aggregate includes a second BBB transport moiety covalently bound to the polycationic polymer scaffold.
44. The method of 42-43, wherein the first and/or second BBB transport moiety includes a peptide.
45. The method of any one of 42-44, wherein the first and/or second BBB transport moiety includes a transferrin receptor ligand.
46. The method of any one of 42-45, wherein the molar ratio of the polycationic polymer scaffold to the first and/or second BBB transport moiety is about 1:1 to about 1:2.
47. The method of any one of 32-46, wherein the aggregate includes a detectable label covalently bound to the polycationic polymer scaffold.
48. The method of any one of 47, wherein the detectable label includes a fluorescent label.
49. The method of any one of 32-48, wherein the polycationic polymer scaffold includes one or more of poly-lysine, poly-arginine, poly-glutamine, poly-amine, poly(diallydimethylammonium chloride) (pDADMAC), and chitosan.
50. The method of any one of 41-49, wherein the second target binding moiety includes a polypeptide.
51. The method of any one of 41-50, wherein the second target binding moiety includes an antibody.
52. The method of any one of 41-49, wherein the second target binding moiety includes an aptamer.
53. The method of any one of 39-52, wherein the molar ratio of the polycationic polymer scaffold to the first target binding moiety is about 1:1 to about 1:5.
54. The method of any one of 32-53, wherein the hydrophilic polymer has a Mw of from about 2 kDa to about 10 kDa.
55. The method of 54, wherein the hydrophilic polymer has a Mw of about 5 kDa.
56. The method of any one of 32-55, wherein the hydrophilic polymer includes a polyethyleneglycol (PEG).
57. The method of any one of 32-56, wherein the interfering RNA includes a short-hairpin RNA (shRNA), a small interfering RNA (siRNA), or a micro-RNA (miRNA).
58. The method of any one of 32-57, wherein the interfering RNA includes a sequence complementary to a portion of a gene transcript for CD-44, CD-44 V6, Sox2, Id-1, Id-3, c-Met, or NCOA3.
59. The method of any one of 32-58, wherein the ratio of nucleic acids to polycationic polymer scaffolds in the nanoparticle is from about 1:100 to about 1:1000.
60. The method of any one of 32-58, wherein the ratio of nitrogen (N) to phosphate (P) in the nanoparticle is from about 1:1 to about 1:100.
61. A method of treating Glioblastoma Multiforme (GBM) in a subject, the method including:
   administering a therapeutically effective amount of a formulation including a plurality of polymeric nanoparticles to a subject in need thereof, each polymeric nanoparticle of the plurality including an aggregate of nucleic acids and polycationic polymer scaffolds, wherein the aggregate includes:
   a polycationic polymer scaffold;
   an amphiphilic peptide covalently bound to the polycationic polymer scaffold;
   a hydrophilic polymer covalently bound to the polycationic polymer scaffold; and
   an interfering RNA or a DNA encoding an interfering RNA bound by ionic-charge interactions to the polycationic polymer scaffold, wherein the interfering RNA includes a sequence complementary to a portion of a gene transcript for CD-44, CD-44 V6, Sox2, Id-1, Id-3, c-Met, or NCOA3.
62. The method of 61, wherein the subject is a mammal.
63. The method of 62, wherein the mammal is in a human.
64. The method of any one of 61-63, wherein the amphiphilic peptide includes a first target binding moiety.
65. The method of 64, wherein the amphiphilic peptide includes chlorotoxin (ClTx).
66. The method of any one of 61-65, wherein the aggregate includes a second target binding moiety covalently bound to the polycationic polymer scaffold.
67. The method of 66, wherein the second target binding moiety includes a first blood brain barrier (BBB) transport moiety.
68. The method of any one of 61-67, wherein the aggregate includes a second BBB transport moiety covalently bound to the polycationic polymer scaffold.
69. The method of 67-68, wherein the first and/or second BBB transport moiety includes a peptide.
70. The method of any one of 67-69, wherein the first and/or second BBB transport moiety includes a transferrin receptor ligand.
71. The method of any one of 67-70, wherein the molar ratio of the polycationic polymer scaffold to the first and/or second BBB transport moiety is about 1:1 to about 1:2.
72. The method of any one of 61-71, wherein the aggregate includes a detectable label covalently bound to the polycationic polymer scaffold.
73. The method of any one of 72, wherein the detectable label includes a fluorescent label.
74. The method of any one of 61-73, wherein the polycationic polymer scaffold includes one or more of poly-lysine, poly-arginine, poly-glutamine, poly-amine, poly(diallydimethylammonium chloride) (pDADMAC), and chitosan.
75. The method of any one of 66-74, wherein the second target binding moiety includes a polypeptide.
76. The method of any one of 66-75, wherein the second target binding moiety includes an antibody.
77. The method of any one of 66-74, wherein the second target binding moiety includes an aptamer.
78. The method of any one of 64-77, wherein the molar ratio of the polycationic polymer scaffold to the first target binding moiety is about 1:1 to about 1:5.
79. The method of any one of 61-78, wherein the hydrophilic polymer has a Mw of from about 2 kDa to about 10 kDa.
80. The method of 79, wherein the hydrophilic polymer has a Mw of about 5 kDa.
81. The method of any one of 61-80, wherein the hydrophilic polymer includes a polyethyleneglycol (PEG).
82. The method of any one of 61-81, wherein the interfering RNA includes a short-hairpin RNA (shRNA), a small interfering RNA (siRNA), or a micro-RNA (miRNA).
83. The method of any one of 61-82, wherein the ratio of nucleic acids to polycationic polymer scaffolds in the nanoparticle is from about 1:100 to about 1:1000.
84. The method of any one of 61-82, wherein the ratio of nitrogen (N) to phosphate (P) in the nanoparticle is from about 1:1 to about 1:100.
85. A method of making a polymeric nanoparticle including:
   combining nucleic acids with covalently-modified polycationic polymer scaffolds, wherein the combining results in condensation of the nucleic acids and formation of a polymeric nanoparticle as set forth in any one of 1-8, 9-15, and 17-29.
86. The method of 85, wherein the method includes covalently bonding the amphiphilic peptide or the first target binding moiety to a polycationic polymer scaffold at a molar ratio of about 5:1 to about 1:1 to provide the covalently-modified polycationic polymer scaffold.
87. The method of 85 or 87, wherein each of the covalently-modified polycationic polymer scaffolds include a second target binding moiety and wherein the method includes covalently bonding the second target binding moiety to a polycationic polymer scaffold at a molar ratio of about 2:1 to about 1:1 to provide the covalently-modified polycationic polymer scaffold.
88. The method of any one of 85-86, wherein the method includes combining the hydrophilic polymer and the polycationic polymer scaffold at a molar ratio of about 4.1.
89. The method of any one of 85-88, wherein the nucleic acids are combined with the polycationic polymer scaffolds at a molar ratio of from about 1:100 to about 1:1000.
90. The method of any one of 85-88, wherein the polycationic polymer scaffolds are combined with the nucleic acids at a respective nitrogen (N) to phosphate (P) ratio of about 1:1 to about 1:100.
91. A polymeric nanoparticle including an aggregate of nucleic acids and polycationic polymer scaffolds, wherein the aggregate includes:
   an amphiphilic peptide covalently bound to a first polycationic polymer scaffold;
   a hydrophilic polymer covalently bound to a second polycationic polymer scaffold; and
   a nucleic acid bound by ionic-charge interactions to the polycationic polymer scaffolds.
92. The polymeric nanoparticle of 91, wherein the first and second polycationic polymer scaffolds are the same.
93. The polymeric nanoparticle of 91, wherein the first and second polycationic polymer scaffolds are distinct polymers.
94. The polymeric nanoparticle of any one of 91-93, wherein the amphiphilic peptide includes a first target binding moiety.
95. The polymeric nanoparticle of 94, wherein the amphiphilic peptide includes chlorotoxin (ClTx).
96. The polymeric nanoparticle of any one of 91-95, including a second target binding moiety covalently bound to the polycationic polymer scaffold.
97. The polymeric nanoparticle of 96, wherein the second target binding moiety includes a first blood brain barrier (BBB) transport moiety.
98. The polymeric nanoparticle of any one of 91-97, including a second BBB transport moiety covalently bound to the polycationic polymer scaffold.
99. The polymeric nanoparticle of 97 or 98, wherein the first and/or second BBB transport moiety includes a peptide.
100. The polymeric nanoparticle of any one of 97-99, wherein the first and/or second BBB transport moiety includes a transferrin receptor ligand.
101. The polymeric nanoparticle of any one of 97-100, wherein the molar ratio of the polycationic polymer scaffold to the first and/or second BBB transport moiety is about 1:1 to about 1:2.
102. The polymeric nanoparticle of any one of 91-101, including a detectable label covalently bound to the polycationic polymer scaffold.
103. The polymeric nanoparticle of any one of 91-102, wherein the detectable label includes a fluorescent label.
104. The polymeric nanoparticle of any one of 91-103, wherein the polycationic polymer scaffold includes one or more of poly-lysine, poly-arginine, poly-glutamine, poly-amine, poly(diallydimethylammonium chloride) (pDADMAC), and chitosan.
105. The polymeric nanoparticle of any one of 91-104, wherein the second target binding moiety includes a polypeptide.
106. The polymeric nanoparticle of any one of 91-105, wherein the second target binding moiety includes an antibody.
107. The polymeric nanoparticle of any one of 91-104, wherein the second target binding moiety includes an aptamer.
108. The polymeric nanoparticle of any one of 91-107, wherein the molar ratio of the polycationic polymer scaffold to the first target binding moiety is about 1:1 to about 1:5.
109. The polymeric nanoparticle of any one of 91-108, wherein the hydrophilic polymer has a Mw of from about 2 kDa to about 10 kDa.
110. The polymeric nanoparticle of 109, wherein the hydrophilic polymer has a Mw of about 5 kDa.
111. The polymeric nanoparticle of any one of 91-110, wherein the hydrophilic polymer includes a polyethyleneglycol (PEG).
112. The polymeric nanoparticle of any one of 91-111, wherein the nucleic acid includes DNA.
113. The polymeric nanoparticle of 112, wherein the DNA encodes an interfering RNA.
114. The polymeric nanoparticle of 113, wherein the interfering RNA includes a short-hairpin RNA (shRNA).
115. The polymeric nanoparticle of 113 or 114, wherein the interfering RNA includes a sequence complementary to a portion of a gene transcript for CD-44, CD-44 V6, Sox2, Id-1, Id-3, c-Met, or NCOA3.
116. The polymeric nanoparticle of any one of 91-111, wherein the nucleic acid includes RNA.
117. The polymeric nanoparticle of 116, wherein the RNA includes interfering RNA.
118. The polymeric nanoparticle of 117, wherein the interfering RNA includes a small interfering RNA (siRNA).
119. The polymeric nanoparticle of 117, wherein the interfering RNA includes a shRNA.
120. The polymeric nanoparticle of 117, wherein the interfering RNA includes a micro-RNA (miRNA).
121. The polymeric nanoparticle of any one of 117-120, wherein the interfering RNA includes a sequence complementary to a portion of a gene transcript for CD-44, CD-44 V6, Sox2, Id-1, Id-3, c-Met, or NCOA3.
122. The polymeric nanoparticle of any one of 91-121, wherein the ratio of nucleic acids to polycationic polymer scaffolds in the nanoparticle is from about 1:100 to about 1:1000.
123. The polymeric nanoparticle of any one of 91-121, wherein the ratio of nitrogen (N) to phosphate (P) in the nanoparticle is from about 1:1 to about 1:100.
124. A method of reducing the expression of a target protein in a cell, the method including
   contacting the cell with a polymeric nanoparticle, the polymeric nanoparticle including an aggregate of nucleic acids and polycationic polymer scaffolds, wherein the aggregate includes:
   an amphiphilic peptide covalently bound to a first polycationic polymer scaffold;
   a hydrophilic polymer covalently bound to a second polycationic polymer scaffold; and
   an interfering RNA or a DNA encoding an interfering RNA bound by ionic-charge interactions to the polycationic polymer scaffolds, wherein the interfering RNA includes a sequence complementary to a portion of a gene transcript for the target protein, and whereby expression of the target protein is reduced relative to expression of the target protein in the absence of the contacting.
125. The method of 124, wherein the first and second polycationic polymer scaffolds are the same.
126. The method of 124, wherein the first and second polycationic polymer scaffolds are distinct polymers.
127. The method of any one of 124, wherein the contacting is in vitro.
128. The method of any one of 124, wherein the contacting is in vivo.
129. The method of 128, wherein the cell is in a mammal.
130. The method of 129, wherein the cell is in a human.
131. The method of any one of 124-130, wherein the cell is a cancer cell.
132. The method of 131, wherein the cancer cell is a glioma cell.
133. The method of any one of 124-132, wherein the amphiphilic peptide includes a first target binding moiety.
134. The method of 133, wherein the amphiphilic peptide includes chlorotoxin (ClTx).
135. The method of any one of 124-134, wherein the aggregate includes a second target binding moiety covalently bound to the polycationic polymer scaffold.
136. The method of 135, wherein the second target binding moiety includes a first blood brain barrier (BBB) transport moiety.
137. The method of any one of 124-136, wherein the aggregate includes a second BBB transport moiety covalently bound to the polycationic polymer scaffold.
138. The method of 136-137, wherein the first and/or second BBB transport moiety includes a peptide.
139. The method of any one of 136-138, wherein the first and/or second BBB transport moiety includes a transferrin receptor ligand.
140. The method of any one of 136-139, wherein the molar ratio of the polycationic polymer scaffold to the first and/or second BBB transport moiety is about 1:1 to about 1:2.
141. The method of any one of 124-140, wherein the aggregate includes a detectable label covalently bound to the polycationic polymer scaffold.
142. The method of any one of 141, wherein the detectable label includes a fluorescent label.
143. The method of any one of 124-142, wherein the polycationic polymer scaffold includes one or more of poly-lysine, poly-arginine, poly-glutamine, poly-amine, poly(diallydimethylammonium chloride) (pDADMAC), and chitosan.
144. The method of any one of 135-143, wherein the second target binding moiety includes a polypeptide.
145. The method of any one of 135-144, wherein the second target binding moiety includes an antibody.
146. The method of any one of 135-143, wherein the second target binding moiety includes an aptamer.
147. The method of any one of 133-146, wherein the molar ratio of the polycationic polymer scaffold to the first target binding moiety is about 1:1 to about 1:5.
148. The method of any one of 124-147, wherein the hydrophilic polymer has a Mw of from about 2 kDa to about 10 kDa.
149. The method of 148, wherein the hydrophilic polymer has a Mw of about 5 kDa.
150. The method of any one of 124-149, wherein the hydrophilic polymer includes a polyethyleneglycol (PEG).
151. The method of any one of 124-150, wherein the interfering RNA includes a short-hairpin RNA (shRNA), a small interfering RNA (siRNA), or a micro-RNA (miRNA).
152. The method of any one of 124-151, wherein the interfering RNA includes a sequence complementary to a portion of a gene transcript for CD-44, CD-44 V6, Sox2, Id-1, Id-3, c-Met, or NCOA3.
153. The method of any one of 124-152, wherein the ratio of nucleic acids to polycationic polymer scaffolds in the nanoparticle is from about 1:100 to about 1:1000.
154. The method of any one of 124-152, wherein the ratio of nitrogen (N) to phosphate (P) in the nanoparticle is from about 1:1 to about 1:100.
155. A method of treating Glioblastoma Multiforme (GBM) in a subject, the method including:
   administering a therapeutically effective amount of a formulation including a
   plurality of polymeric nanoparticles to a subject in need thereof, each polymeric nanoparticle of the plurality including an aggregate of nucleic acids and polycationic polymer scaffolds, wherein the aggregate includes:
   an amphiphilic peptide covalently bound to a first polycationic polymer scaffold;
   a hydrophilic polymer covalently bound to a second polycationic polymer scaffold; and
   an interfering RNA or a DNA encoding an interfering RNA bound by ionic-charge interactions to the polycationic polymer scaffolds, wherein the interfering RNA includes a sequence complementary to a portion of a gene transcript for CD-44, CD-44 V6, Sox2, Id-1, Id-3, c-Met, or NCOA3.
156. The method of 155, wherein the first and second polycationic polymer scaffolds are the same.
157. The method of 155, wherein the first and second polycationic polymer scaffolds are distinct polymers.
158. The method of any one of 155, wherein the subject is a mammal.
159. The method of 158, wherein the mammal is in a human.
160. The method of any one of 155-159, wherein the amphiphilic peptide includes a first target binding moiety.
161. The method of 160, wherein the amphiphilic peptide includes chlorotoxin (ClTx).
162. The method of any one of 155-161, wherein the aggregate includes a second target binding moiety covalently bound to the polycationic polymer scaffold.
163. The method of 162, wherein the second target binding moiety includes a first blood brain barrier (BBB) transport moiety.
164. The method of any one of 155-163, wherein the aggregate includes a second BBB transport moiety covalently bound to the polycationic polymer scaffold.
165. The method of 163-164, wherein the first and/or second BBB transport moiety includes a peptide.
166. The method of any one of 163-165, wherein the first and/or second BBB transport moiety includes a transferrin receptor ligand.
167. The method of any one of 163-166, wherein the molar ratio of the polycationic polymer scaffold to the first and/or second BBB transport moiety is about 1:1 to about 1:2.
168. The method of any one of 155-167, wherein the aggregate includes a detectable label covalently bound to the polycationic polymer scaffold.
169. The method of any one of 168, wherein the detectable label includes a fluorescent label.
170. The method of any one of 155-169, wherein the polycationic polymer scaffold includes one or more of poly-lysine, poly-arginine, poly-glutamine, poly-amine, poly(diallydimethylammonium chloride) (pDADMAC), and chitosan.
171. The method of any one of 162-170, wherein the second target binding moiety includes a polypeptide.
172. The method of any one of 162-171, wherein the second target binding moiety includes an antibody.
173. The method of any one of 162-170, wherein the second target binding moiety includes an aptamer.
174. The method of any one of 160-173, wherein the molar ratio of the polycationic polymer scaffold to the first target binding moiety is about 1:1 to about 1:5.
175. The method of any one of 155-174, wherein the hydrophilic polymer has a Mw of from about 2 kDa to about 10 kDa.
176. The method of 175, wherein the hydrophilic polymer has a Mw of about 5 kDa.
177. The method of any one of 155-176, wherein the hydrophilic polymer includes a polyethyleneglycol (PEG).
178. The method of any one of 155-177, wherein the interfering RNA includes a short-hairpin RNA (shRNA), a small interfering RNA (siRNA), or a micro-RNA (miRNA).
179. The method of any one of 155-178, wherein the ratio of nucleic acids to polycationic polymer scaffolds in the nanoparticle is from about 1:100 to about 1:1000.
180. The method of any one of 155-178, wherein the ratio of nitrogen (N) to phosphate (P) in the nanoparticle is from about 1:1 to about 1:100.
181. A method of making a polymeric nanoparticle including:
   combining nucleic acids with covalently-modified polycationic polymer scaffolds,
wherein the combining results in condensation of the nucleic acids and formation of a polymeric nanoparticle as set forth in any one of 91-100, 102-107, and 109-121.
182. The method of 181, wherein the method includes covalently bonding the amphiphilic peptide or the first target binding moiety to a polycationic polymer scaffold at a molar ratio of about 5:1 to about 1:1 to provide the covalently-modified polycationic polymer scaffold.
183. The method of 181 or 182, wherein each of the covalently-modified polycationic polymer scaffolds include a second target binding moiety and wherein the method includes covalently bonding the second target binding moiety to a polycationic polymer scaffold at a molar ratio of about 2:1 to about 1:1 to provide the covalently-modified polycationic polymer scaffold.
184. The method of any one of 181-183, wherein the method includes combining the hydrophilic polymer and the polycationic polymer scaffold at a molar ratio of about 4:1.
185. The method of any one of 181-184, wherein the nucleic acids are combined with the polycationic polymer scaffolds at a molar ratio of from about 1:100 to about 1:1000.
186. The method of any one of 181-185, wherein the polycationic polymer scaffolds are combined with the nucleic acids at a respective nitrogen (N) to phosphate (P) ratio of about 1:1 to about 1:100.
187. A polymeric nanoparticle including an aggregate of nucleic acids and polycationic polymer scaffolds, wherein the aggregate includes:
   a blood brain barrier (BBB) transport moiety covalently bound to a first polycationic polymer scaffold;
   an amphiphilic peptide and/or target binding moiety covalently bound to a second polycationic polymer scaffold;
   a hydrophilic polymer covalently bound to a third polycationic polymer scaffold;
   an amphiphilic peptide covalently bound to a fourth polycationic polymer scaffold;
   a fifth polycationic polymer scaffold including a copolymer of a polycationic polymer and a hydrophobic polymer (PLPL);
   a sixth polycationic polymer scaffold including a copolymer of a polycationic polymer and a polyethylenimine (LXEI); and
   a nucleic acid bound by ionic-charge interactions to the polycationic polymer scaffolds.
188. The polymeric nanoparticle of 187, wherein two or more of the first through sixth polycationic polymer scaffolds are the same.
189. The polymeric nanoparticle of 187, wherein two or more of the first through the sixth polycationic polymer scaffolds are distinct polymers.
190. The polymeric nanoparticle of 189, wherein each of the first through the sixth polycationic polymer scaffolds is a distinct polycationic polymer scaffold.
191. The polymeric nanoparticle of any one of 188-190, wherein the BBB transport moiety covalently bound to a first polycationic polymer scaffold is a polycationic polymer-bound Transferrin (TPL).
192. The polymeric nanoparticle of any one of 188-191, wherein the amphiphilic peptide and/or target binding moiety covalently bound to a second polycationic polymer scaffold is a polycationic polymer-bound chlorotoxin (CPL).
193. The polymeric nanoparticle of any one of 188-192, wherein the hydrophilic polymer covalently bound to a third polycationic polymer scaffold is a polycationic polymer-bound PEG (PPL).
194. The polymeric nanoparticle of any one of 188-193, wherein the amphiphilic peptide covalently bound to a fourth polycationic polymer scaffold is a polycationic polymer-bound Am1peptide (AmPL).
195. The polymeric nanoparticle of any one of 188-194, wherein the fifth polycationic polymer scaffold including a copolymer of a polycationic polymer and a hydrophobic polymer (PLPL) is a poly-lysine conjugated to lyso-phosphatidylethanolamine.
196. The polymeric nanoparticle of any one of 188-195, including a polycationic polymer scaffold including a detectable label.
197. The polymeric nanoparticle of 196, wherein the detectable label is a fluorescent label.
198. The polymeric nanoparticle of 197, wherein the polycationic polymer scaffold including a detectable label is a polycationic polymer-bound Cy5.5 fluorescent label (CyPL) or polycationic polymer-bound rhodamine (RPL).
199. The polymeric nanoparticle of any one of 188-198, wherein each of the first through the sixth polycationic polymer scaffolds is a distinct polycationic polymer scaffold, and wherein 2% to 12% of the total number of polycationic polymer scaffolds which make up the nanoparticle are polycationic polymer scaffolds in which the BBB transport moiety is covalently bound to the first polycationic polymer scaffold.
200. The polymeric nanoparticle of any one of 188-199, wherein each of the first through the sixth polycationic polymer scaffolds is a distinct polycationic polymer scaffold, and wherein 3% to 10% of the total number of polycationic polymer scaffolds which make up the nanoparticle are polycationic polymer scaffolds in which the amphiphilic peptide and/or target binding moiety is covalently bound to the second polycationic polymer scaffold.
201. The polymeric nanoparticle of any one of 188-200, wherein each of the first through the sixth polycationic polymer scaffolds is a distinct polycationic polymer scaffold, and wherein 14% to 35% of the total number of polycationic polymer scaffolds which make up the nanoparticle are polycationic polymer scaffolds in which the hydrophilic polymer is covalently bound to the third polycationic polymer scaffold.
202. The polymeric nanoparticle of any one of 188-201, wherein each of the first through the sixth polycationic polymer scaffolds is a distinct polycationic polymer scaffold, and wherein 25% to 35% of the total number of polycationic polymer scaffolds which make up the nanoparticle are polycationic polymer scaffolds in which the amphiphilic peptide is covalently bound to the fourth polycationic polymer scaffold.
203. The polymeric nanoparticle of any one of 188-202, wherein each of the first through the sixth polycationic polymer scaffolds is a distinct polycationic polymer scaffold, and wherein <1% to 11% of the total number of polycationic polymer scaffolds which make up the nanoparticle are the fifth polycationic polymer scaffold including a copolymer of a polycationic polymer and a hydrophobic polymer (PLPL).
204. The polymeric nanoparticle of any one of 188-203, wherein each of the first through the sixth polycationic polymer scaffolds is a distinct polycationic polymer scaffold, and wherein 25% to 35% of the total number of polycationic polymer scaffolds which make up the nanoparticle are the sixth polycationic polymer scaffold including a copolymer of a polycationic polymer and a polyethylenimine (LXEI).
205. The polymeric nanoparticle of any one of 188-204, wherein each of the first through the sixth polycationic polymer scaffolds is a distinct polycationic polymer scaffold, wherein the nanoparticle includes a polycationic polymer scaffold including a detectable label, and wherein 1% to 3% of the total number of polycationic polymer scaffolds which make up the nanoparticle are the polycationic polymer scaffold including the detectable label.
206. The polymeric nanoparticle of any one of 188-205, wherein one or more of the polycationic polymer scaffolds includes one or more of poly-lysine, poly-arginine, poly-glutamine, poly-amine, poly(diallydimethylammonium chloride) (pDADMAC), and chitosan.
207. The polymeric nanoparticle of any one of 188-206, wherein the nucleic acid includes DNA.
208. The polymeric nanoparticle of 207, wherein the DNA encodes an interfering RNA.
209. The polymeric nanoparticle of 208, wherein the interfering RNA includes a short-hairpin RNA (shRNA).
210. The polymeric nanoparticle of 207 or 208, wherein the interfering RNA includes a sequence complementary to a portion of a gene transcript for CD-44, CD-44 V6, Sox2, Id-1, Id-3, c-Met, or NCOA3.
211. The polymeric nanoparticle of any one of 188-206, wherein the nucleic acid includes RNA.
212. The polymeric nanoparticle of 211, wherein the RNA includes interfering RNA.
213. The polymeric nanoparticle of 212, wherein the interfering RNA includes a small interfering RNA (siRNA).
214. The polymeric nanoparticle of 212, wherein the interfering RNA includes a shRNA.
215. The polymeric nanoparticle of 212, wherein the interfering RNA includes a micro-RNA (miRNA).
216. The polymeric nanoparticle of any one of 212-215, wherein the interfering RNA includes a sequence complementary to a portion of a gene transcript for CD-44, CD-44 V6, Sox2, Id-1, Id-3, c-Met, or NCOA3.
217. A method of reducing the expression of a target protein in a cell, the method including contacting the cell with a polymeric nanoparticle according to any one of 187-206, wherein the nucleic acid is an interfering RNA or a DNA encoding an interfering RNA bound by ionic-charge interactions to the polycationic polymer scaffolds, wherein the interfering RNA includes a sequence complementary to a portion of a gene transcript for the target protein, and whereby expression of the target protein is reduced relative to expression of the target protein in the absence of the contacting.
218. A method of treating Glioblastoma Multiforme (GBM) in a subject, the method including: administering a therapeutically effective amount of a formulation including a plurality of polymeric nanoparticles to a subject in need thereof, wherein each polymeric nanoparticle is a polymeric nanoparticle according to any one of 187-206, and wherein the nucleic acid is an interfering RNA or a DNA encoding an interfering RNA bound by ionic-charge interactions to the polycationic polymer scaffolds, wherein the interfering RNA includes a sequence complementary to a portion of a gene transcript for CD-44, CD-44 V6, Sox2, Id-1, Id-3, c-Met, or NCOA3.
219. A method of introducing a nucleic acid into a prostate cancer cell, the method including contacting the cell with a polymeric nanoparticle including an aggregate of nucleic acids and polycationic polymer scaffolds, wherein the aggregate includes:
   a first polycationic polymer scaffold including a polycationic polymer-bound Transferrin (TPL);
   a hydrophilic polymer covalently bound to a second polycationic polymer scaffold;
   an amphiphilic peptide covalently bound to a third polycationic polymer scaffold to provide a polycationic polymer scaffold-bound amphiphilic peptide, wherein the nanoparticle includes greater than 6E+13 of the polycationic polymer scaffold-bound amphiphilic peptide;
   a fourth polycationic polymer scaffold including a copolymer of a polycationic polymer and a hydrophobic polymer (PLPL);
   a fifth polycationic polymer scaffold including a copolymer of a polycationic polymer and a polyethylenimine (LXEI); and
   a nucleic acid bound by ionic-charge interactions to the polycationic polymer scaffolds.
220. The method of 219, wherein the hydrophilic polymer covalently bound to a second polycationic polymer scaffold is a polycationic polymer-bound PEG (PPL).
221. The method of 219 or 220, wherein the amphiphilic peptide covalently bound to a third polycationic polymer scaffold is a polycationic polymer-bound Am1peptide (AmPL).
222. The method of any one of 219-221, wherein the fourth polycationic polymer scaffold including a copolymer of a polycationic polymer and a hydrophobic polymer (PLPL) is a poly-lysine conjugated to lyso-phosphatidylethanolamine.
223. The method of any one of 219-222, wherein the nanoparticle includes a polycationic polymer scaffold including a detectable label.
224. The method of 223, wherein the detectable label is a fluorescent label.
225. The method of 224, wherein the polycationic polymer scaffold including a detectable label is a polycationic polymer-bound Cy5.5 fluorescent label (CyPL) or polycationic polymer-bound rhodamine (RPL).
226. The method of any one of 219-225, wherein one or more of the polycationic polymer scaffolds includes one or more of poly-lysine, poly-arginine, poly-glutamine, poly-amine, poly(diallydimethylammonium chloride) (pDADMAC), and chitosan.
227. The method of any one of 219-226, wherein the nucleic acid includes DNA.
228. The method of 227, wherein the DNA encodes an interfering RNA.
229. The method of 228 wherein the interfering RNA includes a short-hairpin RNA (shRNA).
230. The method of any one of 219-226, wherein the nucleic acid includes RNA.
231. The method of 230, wherein the RNA includes interfering RNA.
232. The method of 231, wherein the interfering RNA includes a small interfering RNA (siRNA).
233. The method of 231, wherein the interfering RNA includes a shRNA.
234. The method of 231, wherein the interfering RNA includes a micro-RNA (miRNA).
235. A method of treating prostate cancer in a subject, the method including: administering a therapeutically effective amount of a formulation including a plurality of polymeric nanoparticles to a subject in need thereof, wherein each polymeric nanoparticle is a polymeric nanoparticle as recited in the method of any one of 219-234, and wherein the nucleic acid is an interfering RNA or a DNA encoding an interfering RNA bound by ionic-charge interactions to the polycationic polymer scaffolds, wherein the interfering RNA includes a sequence complementary to a portion of a gene transcript for a gene which is upregulated in prostate cancer.
236. The method of 235, wherein the gene is a gene encoding a transcription factor.
237. The method of 235, wherein the gene is selected from CD44, PSMA, PD-L1, and PD-1.
238. A method of treating prostate cancer in a subject, the method including: administering a therapeutically effective amount of a formulation including a plurality of polymeric nanoparticles to a subject in need thereof, wherein each polymeric nanoparticle is a polymeric nanoparticle as recited in the method of any one of 219-226, and wherein the nucleic acid is an interfering RNA or a DNA encoding an interfering RNA bound by ionic-charge interactions to the polycationic polymer scaffolds, wherein the interfering RNA includes a microRNA which is downregulated in prostate cancer or which targets a gene transcript for a gene which is upregulated in prostate cancer.
239. The method of 238, wherein the microRNA is selected from mir-34a, mir-205, mir-18, mir-101, and mir-7.
240. The method of 238, wherein the microRNA is a microRNA that targets a component of the PD-L1/PD-1 pathway.
241. A method of introducing a nucleic acid into a melanoma cell, the method including contacting the cell with a polymeric nanoparticle including an aggregate of nucleic acids and polycationic polymer scaffolds, wherein the aggregate includes:
   a first polycationic polymer scaffold including a polycationic polymer-bound Transferrin (TPL);
   a hydrophilic polymer covalently bound to a second polycationic polymer scaffold;
   an amphiphilic peptide covalently bound to a third polycationic polymer scaffold;
   a fourth polycationic polymer scaffold including a copolymer of a polycationic polymer and a hydrophobic polymer (PLPL);
   a fifth polycationic polymer scaffold including a copolymer of a polycationic polymer and a polyethylenimine (LXEI); and
   a nucleic acid bound by ionic-charge interactions to the polycationic polymer scaffolds.
242. The method of 241, wherein the hydrophilic polymer covalently bound to a second polycationic polymer scaffold is a polycationic polymer-bound PEG (PPL).
243. The method of 241 or 242, wherein the amphiphilic peptide covalently bound to a third polycationic polymer scaffold is a polycationic polymer-bound Am1peptide (AmPL).
244. The method of any one of 241-243, wherein the fourth polycationic polymer scaffold including a copolymer of a polycationic polymer and a hydrophobic polymer (PLPL) is a poly-lysine conjugated to lyso-phosphatidylethanolamine.
245. The method of any one of 241-244, wherein the nanoparticle includes a polycationic polymer scaffold including a detectable label.
246. The method of 245, wherein the detectable label is a fluorescent label.
247. The method of 246, wherein the polycationic polymer scaffold including a detectable label is a polycationic polymer-bound Cy5.5 fluorescent label (CyPL) or polycationic polymer-bound rhodamine (RPL).
248. The method of any one of 241-247, wherein one or more of the polycationic polymer scaffolds includes one or more of poly-lysine, poly-arginine, poly-glutamine, poly-amine, poly(diallydimethylammonium chloride) (pDADMAC), and chitosan.
249. The method of any one of 241-248, wherein the nucleic acid includes DNA.
250. The method of 249, wherein the DNA encodes an interfering RNA.
251. The method of 250 wherein the interfering RNA includes a short-hairpin RNA (shRNA).
252. The method of any one of 241-248, wherein the nucleic acid includes RNA.
253. The method of 252, wherein the RNA includes interfering RNA.
254. The method of 253, wherein the interfering RNA includes a small interfering RNA (siRNA).
255. The method of 253, wherein the interfering RNA includes a shRNA.
256. The method of 253, wherein the interfering RNA includes a micro-RNA (miRNA).
257. A method of treating melanoma in a subject, the method including: administering a therapeutically effective amount of a formulation including a plurality of polymeric nanoparticles to a subject in need thereof, wherein each polymeric nanoparticle is a polymeric nanoparticle as recited in the method of any one of 241-256, and wherein the nucleic acid is an interfering RNA or a DNA encoding an interfering RNA bound by ionic-charge interactions to the polycationic polymer scaffolds, wherein the interfering RNA includes a sequence complementary to a portion of a gene transcript for a gene which is upregulated in melanoma.
258. The method of 257, wherein the gene is selected from BPTF, CD44, a Sox gene, PD-L1 and PD-1.
259. A method of treating prostate cancer in a subject, the method including: administering a therapeutically effective amount of a formulation including a plurality of polymeric nanoparticles to a subject in need thereof, wherein each polymeric nanoparticle is a polymeric nanoparticle as recited in the method of any one of 241-248, and wherein the nucleic acid is an interfering RNA or a DNA encoding an interfering RNA bound by ionic-charge interactions to the polycationic polymer scaffolds, wherein the interfering RNA includes a microRNA which is downregulated in melanoma or which targets a gene transcript for a gene which is upregulated in melanoma.
260. The method of 259, wherein the microRNA is selected from mir-34, mir-18, mir-7, mir-101, and mir-7.
261. The method of 259, wherein the microRNA is a microRNA that targets a component of the PD-L1/PD-1 pathway.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Example 1

### Preparation of Polymeric Nanoparticles

Polymeric nanoparticles including siRNA or plasmids capable of expressing shRNA were prepared as follows: Generally, the synthetic polymeric polycation (poly-lysine (PL₂₃₅), Mw=38 kDa) was covalently conjugated with N-terminal modified Transferrin (Tf), a ligand binding to transferrin cell surface receptor, and the conjugate was purified by column fractionation forming Tf-PL₂₃₅. Each polymeric polycation (PL₂₃₅) molecule carried 1-2 subunits of receptor ligand. The same polymeric polycation was also covalently conjugated to 1-5 molecules of modified Chlorotoxin peptide (MCP). The fractionated Tf-PL₂₃₅ was successively thiolated by PDP activation and purified by size exclusion chromatography (SEC). Following quantification of the degree of modification, Tf-PL₂₃₅-SH was reacted with thiolated ClTx (MCMPCFTTDHQMARKCDDCCGGKGRGKCYGPQCLCR) or a random 36-aa peptide (RP). Purified thiolated Tf-PL₂₃₅ also were covalently conjugated to PEG (Mw=5000 Daltons) by reaction with propionaldehyde-PEG (ald-PEG) in a PEGylation reaction catalyzed by cyanoborohydride at exact stoichiometries.

More specifically, the following steps were performed:

Step I: Crosslinking of n-Terminal Activated Tf to Pyridylthiol Activated PL:

Activation of Tf and PL prior to cross-linking: In this procedure Tf and PL were activated into Pyridyldithiol-activated forms by reacting with 20 mM solution of N-succinimidyl 3-(2-pyridyldithio) propionate (Mw=312.37; arm length=6.8 Å (SPDP; Thermo Scientific). To do so, Tf and PL were resuspended in PBSE (20 mM Potassium Phosphate, 150 mM NaCl, 1 mM EDTA, 0.02% sodium Azide, pH 7.5). Tf was Incubated with SPDP reagent in a 1:4 (Tf:SPDP) molar ratio for 30 minutes at room temperature. At completion, unreacted SPDP were removed and Pyridyldithiol-activated Tf and PL were desalted by size exclusion chromatography (SEC) in a 7 kDa MWCO column (Zeba Spin Columns, Thermo Scientific) as per manufacturer's protocol. The efficiency of SPDP modification was assessed by Pyridine-2-Thione Assay as follows: Dilute 100 μL of SPDP-modified and desalted protein to 1 mL with PBS. Measure and record the absorbance at 343 nm of the protein sample compared to PBSE blank (test in triplicate). Add 10 μL of 15 mg/mL DTT to the 1 mL SPDP-modified protein sample, mix. After exactly 15 minutes, measure and record the absorbance at 343-nm of the reduced sample. Calculate the change in absorbance: ΔA343=(Ave. A343 after DTT)−(Ave. A343 before DTT). Calculate molar ratio of SPDP to protein using the following equation:

$$\frac{Δ A}{8080} \times \frac{Mw of Protein}{\frac{mg}{mL} of Protein} = mole of SPDP per mole of protein ,$$

where the value 8080 reflects the extinction coefficient for pyridine-2-thione at 343 nm: 8.08×10³ M⁻¹ cm⁻¹.

N-terminal Pyridine-2-thione group was removed from Tf using reducing agent, 2-Mercaptoethylamine (2-MEA; CAS #156-57-0; Mw=113.61) by incubation at 50 mM final concentration of 2-MEA in PBSE.

After removing the reducing agent using a desalting column, the resulting sulfhydryl-modified Tf and the SPDP-modified PL were incubated together to make the PL-Tf (1:1) conjugate.

Step II: Crosslinking of Cy5.5 to PL (1:1) and Purification:

1 mg PL was dialyzed against 0.1M NaHCO3 (pH8.3) overnight. The buffer exchanged polymer was reacted with Cy5.5-NHS ester (Mw=592 daltons; Lumiprobe Inc), at 1:1 molar ratio forming Cy5.5 labeled PL.

Step III: Crosslinking of ClTx to Cy-Tf-PL:

Pyridylthiol-activated ClTx was prepared by using SPDP activation procedure listed above and purified by SEC. Pyridylthiol activation was tested by Pyridine-2-thione assay detailed above. 2-MEA reaction was used to produce sulfhydryl modified ClTx (SH-ClTx). N-terminal SH-ClTx was purified using SEC via P10 column (GE healthcare). Column purified SH-ClTx was reacted with Pyridylthiol-activated Tf-PL at 1:1 molar ratio. The resultant product was tested by non-denaturing gel electrophoresis and purified by Amicon column filtration (MWCO=55 kDa).

Step IV: Crosslinking SH-PEG to Cy-ClTx-Tf-PL:

SH-PEG was reacted with Pyridylthiol-activated Cy-ClTx-Tf-PL at a 4:1 molar ratio and purified by amicon column filtration (MWCO=55 KDa), forming Cy-PEG-ClTx-Tf-PL.

Step V. Nucleic Acid Condensation Forming Nanoparticles:

Nucleic acids, siRNA against cMET, CD44, Sox2, Id-1, and Id-3, or plasmids expressing shRNA against cMET, CD44, Sox2, Id-1, and Id-3, were condensed with Cy-PEG-ClTx-Tf-PL monomers (i.e., Cy-PEG-ClTx-Tf-PL NP polymer scaffold subunits) at a nitrogen to phosphate (N:P) charge ratio of 32 and purified by amicon column filtration (MWCO=100 KDa). Select purified nanoparticles were tested in-vivo as detailed in Example 3 below.

Examples 2-3 below utilized NPs prepared according to the above method.

### Example 2

### In-Vitro Delivery to Primary GBM Cells

Stabilized and targeted ClTx-Cy5.5-ClTx-Tf-PEG-PL₂₃₅/pGFP-based nanoparticles were prepared generally as described above with the exception that plasmid DNA encoding green fluorescent protein (GFP) was included as the nucleic acid.

Materials and Methods:

Several NP with different attributes and conjugate stoichiometries were designed to express eGFP gene carried on a mammalian expression plasmid, where the eGFP expression was driven by hCMV promoter/enhancer. The formulated nanoparticles included conjugated tissue targeting surface markers such as ClTx and T_{f}, and covalently attached PEG₅₀₀₀ for in vivo stability. Stoichiometric ratios of surface moieties and scaffolds optimized for delivery of GFP plasmid DNA and siRNA were tested in vitro. An optimized formulation is detailed above (see description of NP formulation) where each monomer (i.e., NP polymer scaffold subunit) contained a PL₂₃₅ polymeric lysine scaffold covalently bonded to PEG₅₀₀₀ providing it the ability to evade host immune mechanisms, N-terminal Tf ligand for passage through the BBB, and surface attached ClTx to specifically target GBM cells. In vitro delivery studies were performed in cultured U87 GBM primary cells obtained from ATCC and grown to 80% confluence in 24-well plates at 37° C. For intracellular tracking or for tissue localization, covalently attached far-red fluorescent dye Cy5.5 was also included. 48-hours following delivery cells were tested for GFP delivery by flow cytometric analysis of GFP expression. For FACS analysis, cells were detached by physical means and analyzed using a FACS sorter (Becton Dickinson). Microscopic analysis was performed using fluorescence microscope fitted with GFP excitation and emission filters.

Results:

5×10⁶ or 1×10⁷ NP prepared from 10⁴ monomers (i.e., NP polymer scaffold subunits) of ClTx-Cy5.5-Tf-PEG-PL235/pGFP demonstrated minimum cellular toxicity. FACS analysis and microscopic visualization demonstrated that while 5×10⁶NP delivery gave 1-2% efficiency of GFP expression, 1×10⁷ NP demonstrated consistently higher (12-25%) delivery efficiency (FIG. 2). Overall, the results demonstrated minimum cellular toxicity and high expression of GFP in cultured GBM cells.

### Example 3

### In-Vivo Delivery of Interfering RNA to GBM Cells

GBM cultures enriched in glioma stem like cells (referred to as GSC1 and GSC2) which express Id-1 and can recapitulate the disease in vivo when intracranially implanted in nude mice were used to test interfering RNA containing nanoparticles in vivo.

Materials and Methods:

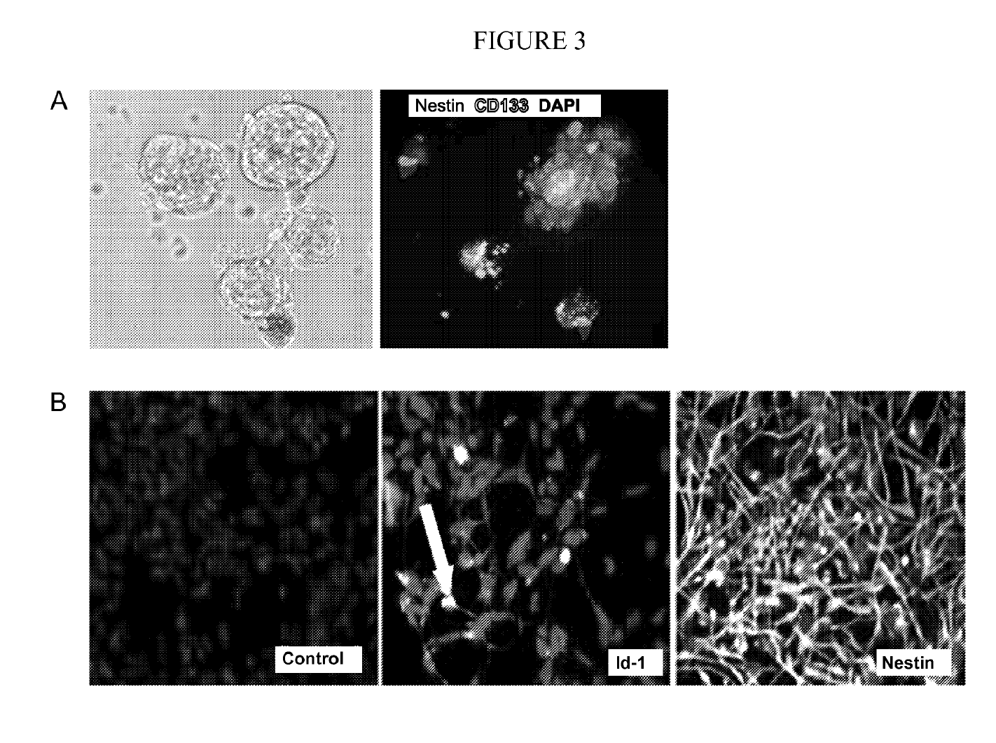

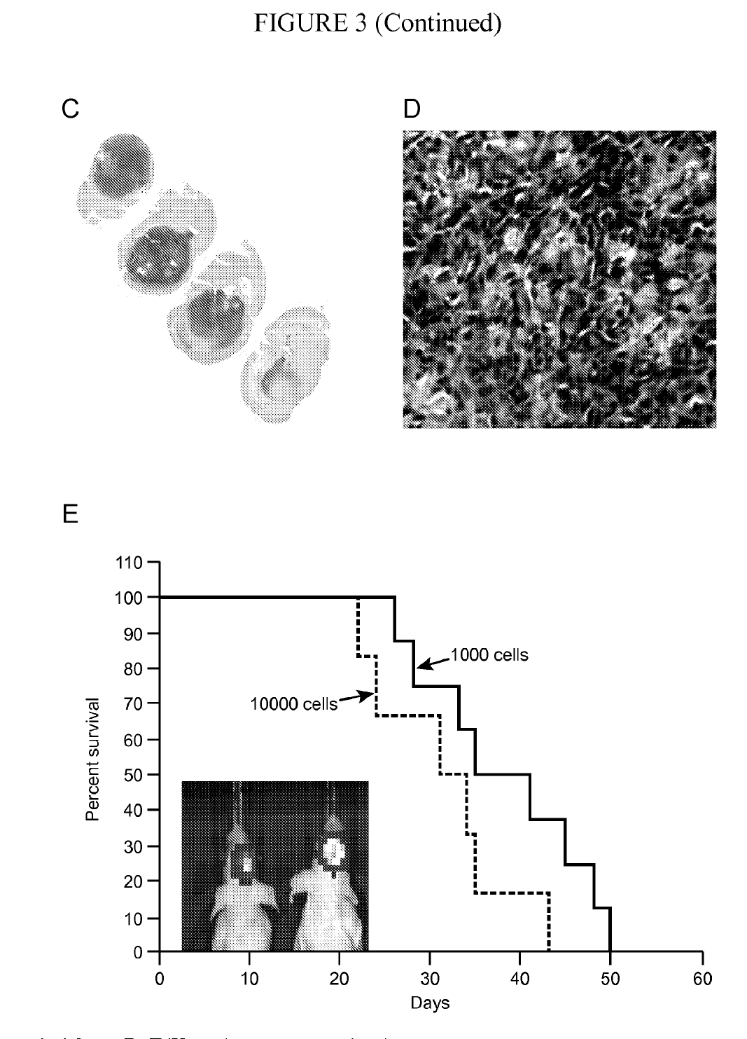

Primary GBM tissues were sorted for stem cell markers and cultured in neurosphere conditions. FIG. 3, Panel A, left, provides a phase photomicrograph of the cultures. FIG. 3, Panel A, right, shows results for the immunofluorescent detection of Nestin and CD133 (two stem cell markers). Immuno-fluorescence analysis of primary GSC cells showed they were positive for Id-1 (FIG. 3, Panel B, middle) and nestin (FIG. 3, Panel B, right). The results for control IgG are shown in FIG. 3, Panel B, left. Cells were counterstained with DAPI.

An in vivo GBM mouse model was produced by intracranial injection of GSC1 and GSC2 cells. The cells were modified to express luciferase, which enables the measurement of tumor development in vivo, in real time.

FIG. 3, Panel C, shows results for H&E staining of intracranially grown tumors derived from primary GSC. FIG. 3, Panel D, provides a high magnification demonstrating its histological resemblance to human GBM. FIG. 3, Panel E, shows results for the injection of luciferase labeled GSC1 cells in nude mice at two cell densities. Tumor growth was monitored in real time using the IVIS Lumina instrument and survival was recorded.

10⁷ or 10⁸ interfering RNA containing nanoparticles were injected into GBM mice by tail-vein injection in a total saline volume of 200 μL. The nanoparticles were prepared as discussed above using a Sox2 siRNA pool obtained from Thermo Scientific-Dharmacon as the nucleic acid active agent.

Results:

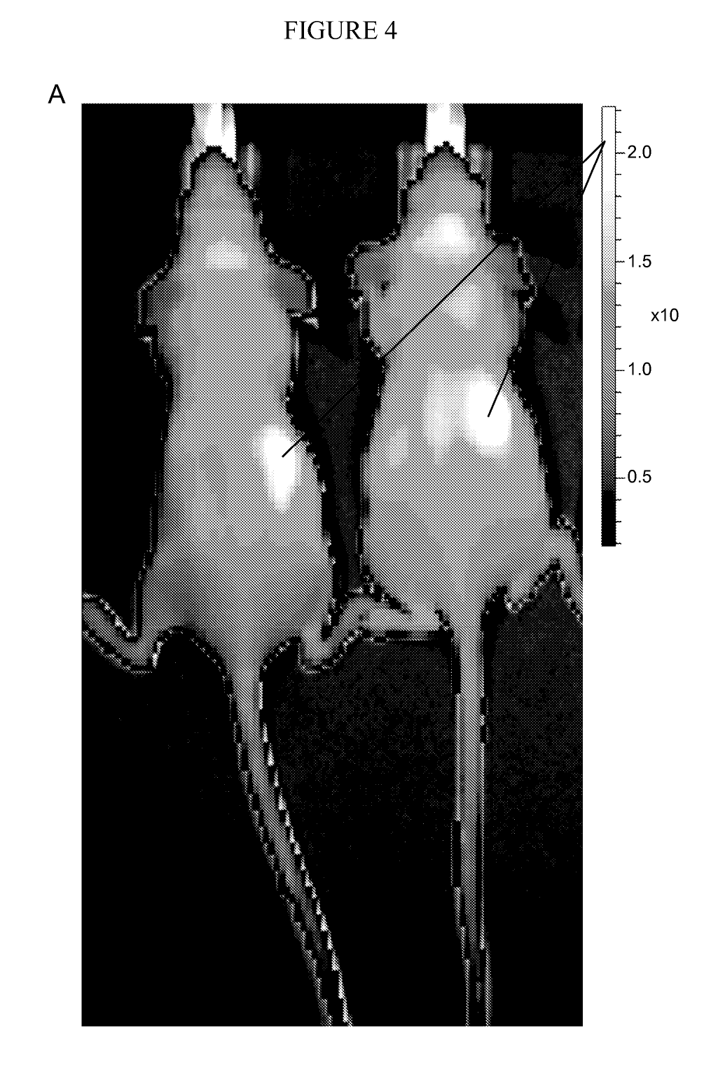

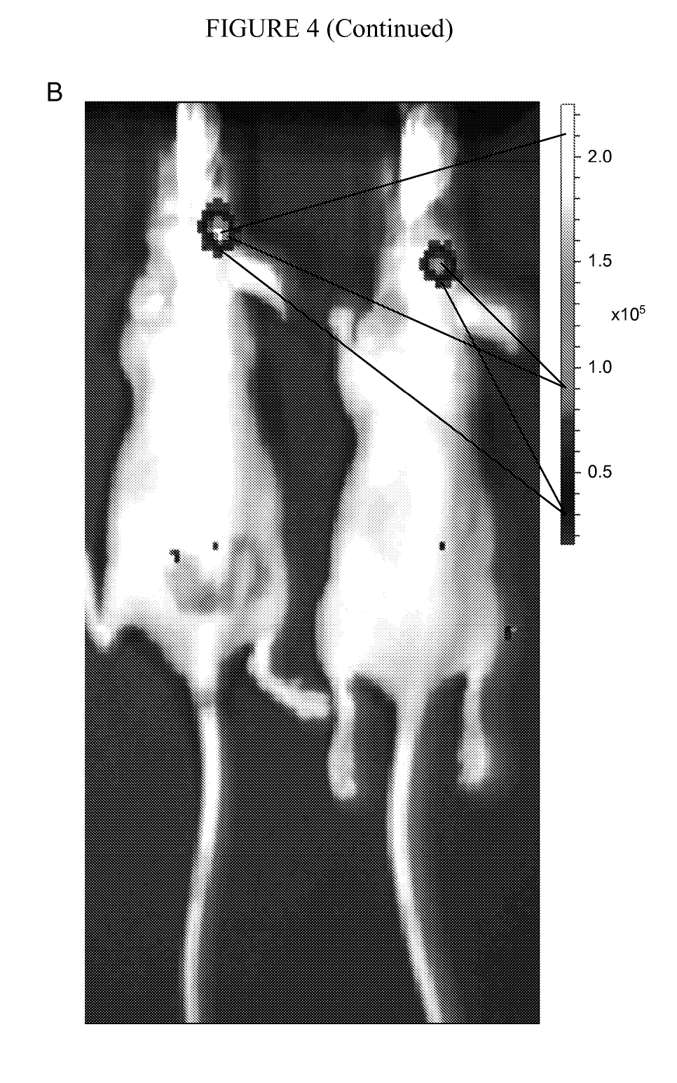

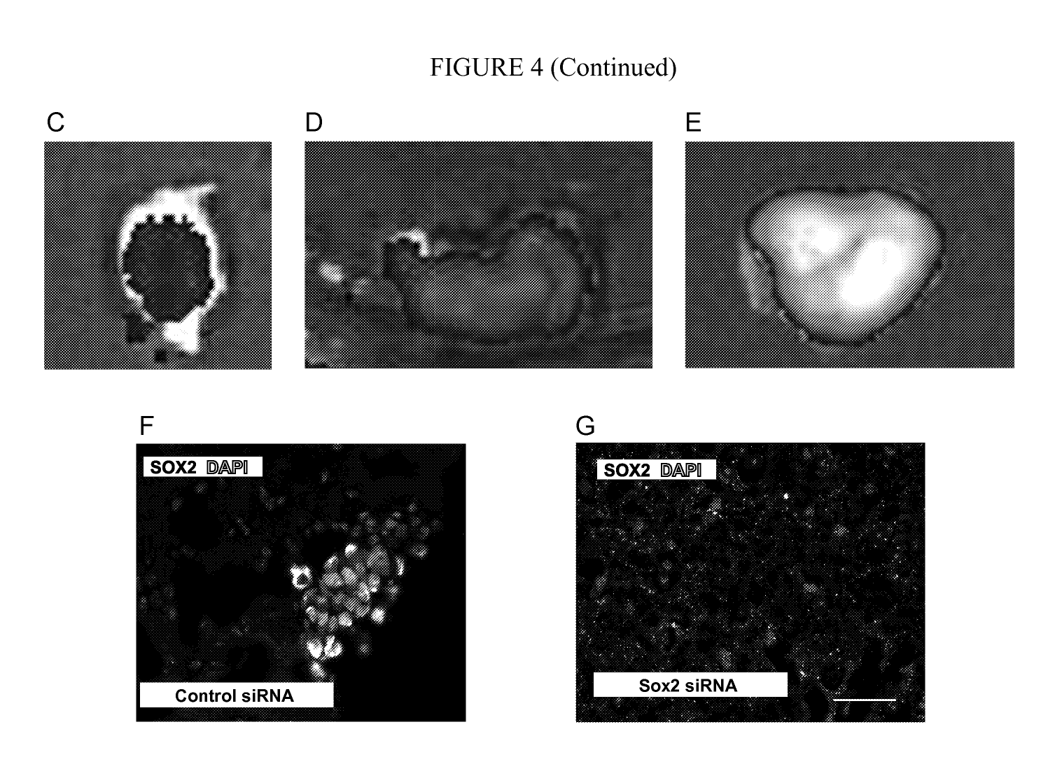

FIG. 4 provides in-vivo results showing that ClTx provides tissue specific targeting of nanoparticles to GBM and inhibition of Sox2 expression in a GBM following i.v. delivery of nanoparticles. Seventy two hours after i.v. delivery of nanoparticles, mice were monitored by whole body scanning for Cy5.5 signal (Panels A and B). Tissue distribution following delivery of ClTx-Cy5.5-Tf-PEG-PL₂₃₅/SOX2 siRNA (Panel A, mouse on the right) was directly compared with the same nanoparticle using control siRNA (Panel A, left mouse). Luminescence measurements are shown in Panel B. Brain specific nanoparticle delivery was prominent in the presence of ClTx. Imaging of the brain, kidneys and liver following nanoparticle delivery (Panels C, D and E). 72 hours following i.v. delivery of nanoparticles, brains were flash frozen and processed for immunofluorescence for Sox2 (Panels F and G). Representative photomicrographs are shown. Panel F-control siRNA, Panel G-Sox2 siRNA. Counterstaining with DAPI. Bar=100 μm.

### Example 4

### Additional Method for the Preparation of Polymeric Nanoparticles

### NP Construction:

NP Constituents:

NPs were assembled as a combination composed of specific ratios of specific examples of the following NP polymeric components (i.e., NP polymer scaffold subunits): 1) AmPL, 2) LXEI, 3) CPL, 4) PPL, 5) TPL, 6) PLPL and 7) CyPL or RPL (fluorescent label). Detailed composition of an example of each NP polymeric component (i.e., NP polymer scaffold subunit) type is described below.
1) AmPL: is a conjugate of PL₂₃₅ polymer (PL) with custom produced amphiphilic peptide 1 (Am1) forming AmPL. AmPL accounts for 28% to 35% of the NP polymeric components (i.e., NP polymer scaffold subunits) which make up the NP.
2) LXEI: is a copolymer of PL and polyethylenimine (PEI). It is a 1:1 to 1:2 conjugate of PL (L) cross-linked (X) to PEI (2 KDa)(EI), where one amine on PL₂₃₅ is specifically conjugated to an amine on PEI (2 KDa) as a thioether linked conjugate. LXEI accounts for 25% to 35% of the NP polymeric components (i.e., NP polymer scaffold subunits) which make up the NP.
3) CPL: is NP polymeric component (i.e., NP polymer scaffold subunit) containing PL conjugated to Chlorotoxin peptide at 1:1 to 1:4 ratio. CPL accounts for 3% to 10% of the NP polymeric components (i.e., NP polymer scaffold subunits) which make up the NP.
4) PPL: is PL₂₃₅ NP polymeric component (i.e., NP polymer scaffold subunit) conjugated to PEG (Mw=5000 KDa) at 1 to 4 PEG per PL₂₃₅. PPL accounts for 14% to 30% of the NP polymeric components (i.e., NP polymer scaffold subunits) which make up the NP.
5) TPL: Transferrin-PL is a conjugate of holo human transferrin (86 KDa) carrying 2 Fe atoms. The conjugate was synthetically formed by stoichiometric conjugation of N-terminal human holo-transferrin with thiolated PL generated in a thiolation reaction as described below. TPL accounts for 2% to 12% of the NP polymeric components (i.e., NP polymer scaffold subunits) which make up the NP.
6) PLPL: Is a synthetic conjugate PL₂₃₅ and sub CMC amount of Phophatidylethanolamine (lyso-form) via N-terminal amine and ethanolamine moieties. PLPL accounts for <1% to 11% of final NP.
7) CyPL or RPL: is a fluorescent conjugate where PL is crosslinked to either Cy5.5 (far-red fluorescent dye for in-vivo monitoring) or Rhodamine (R, for in-vitro monitoring). CyPL or RPL formed <1% to 3% of the NP polymeric components (i.e., NP polymer scaffold subunits) which make up the NP.

AmPL Formulation:

Amphiphilic peptidel (Am1), was synthetically produced and purified by HPLC with the following amino-acid sequence: NH2-GIGAVLKVLTTGLPALISWIKRKRHHC-COOH. Am1 was cross-linked to PL-SH by disulfide bonding. Am1 was conjugated to PL, which was activated prior to cross-linking, whereby, N-terminal amino or 1 to 2 ε-amino groups was converted to Pyridyldithiol activated PL by reacting to SPDP (N-succinimidyl 3-(2-pyridyldithio) propionate) as detailed below. The pyridyldithiol activated PL was further reduced by TCEP (Tris(2-carboxyethyl)phosphine HCl) in PBSE to form sulfhydryl activated PL (PL-SH). Crosslinking reaction was performed in PBSE where, 280 μg Am1 was thoroughly mixed with 600 μg PL-SH for 48 hours at room temperature forming AmPL. The final formed AmPL was purified by SEC through MWCO10K column. The stoichiometric AmPL formation was confirmed by protein quantification by spectrophotometry and by performing NDN assays described below.

SPDP Activation of PL:

PL was activated into Pyridyldithiol-activated forms by reacting with SPDP. Ten to 100 mg PL was dissolved in 5- to 10-mL DMSO and 50 mM TEA (final concentration) and reacted with 20 mM solution of N-succinimidyl 3-(2-pyridyldithio) propionate (Mw=312.37; arm length=6.8 Å (SPDP; Thermo Scientific) in DMSO for 2-hrs at room temperature producing PL-PDP (PDP) which was diluted with 40-mL DMSO and purified away from unreacted SPDP using stir-cell 10 KDa MWCO concentrator (Millipore Inc.) under inert gas pressure (200 mPa) to 2- to 5-mL final volume and supplemented with 3× volume double distilled-sterile water (DDS-water) and mixed thoroughly. The mixture was frozen and lyophilized in a custom constructed lyophilizer and resuspended in 10-mL DDS water. PDP was further purified by size exclusion chromatography to ensure >30 and <70 KDa polymeric size of final PL₂₃₅. PDP reaction efficiency was quantified by Pyridine-2-Thione assay (described below) and PL₂₃₅ concentration was quantified by NDN assay (described below). The activated PL was reacted with molar excess of TCEP in PBSE to obtain PL-SH.

Pyridine-2-Thione Assay:

The efficiency of SPDP modification was assessed by Pyridine-2-Thione Assay as follows: Dilute 100 μL of SPDP-modified and desalted protein to 1 mL with PBS. Measure and record the absorbance at 343 nm of the protein sample compared to PBSE blank (test in triplicate). Add 10 μL of 15 mg/mL DTT to the 1 mL SPDP-modified protein sample, mix. After exactly 15 minutes, measure and record the absorbance at 343-nm of the reduced sample. Calculate the change in absorbance: ΔA343=(Ave. A343 after DTT)−(Ave. A343 before DTT). Calculate molar ratio of SPDP to protein using the following equation:

$$\frac{Δ A}{8080} \times \frac{Mw of Protein}{\frac{mg}{mL} of Protein} = mole of SPDP per mole of protein ,$$

where the value 8080 reflects the extinction coefficient for pyridine-2-thione at 343 nm: 8.08×10³ M⁻¹ cm⁻¹. Minimum acceptable SPDP mole ratio per mole PL for PL-SH was 1.5.

NDN Assay: NDN assay was used to quantify each NP polymeric component (i.e., NP polymer scaffold subunit). NDN assay solutions produced and stored in inert gas were as follows: Reagent N: 0.08 g Ninhydrin in 3 mL DMSO, 0.012 g Hydrindantin and 1 mL of 4M LiOAc (pH 5.2) was used to analyze all samples and test against a standard. After preparing a standard curve with PL in 0.05% glacial acetic acid, each formulation was quantified against standard curve within the linear range of the assay.

LXEI Co-Polymer Synthesis:

LXEI is a copolymer produced by crosslinking PL (L) to 2 kDa linear polyethyleneimine polymer (PEI) at a 1:1 to 1:4 ratio. Specifically, 1 mg L was crosslinked to 80 μg EI, a 1:2 molar ratio, in a single vessel in BE buffer (pH 8) by reacting together with 2× molar excess of 2-Iminothiolane hydrochloride (Sigma). The crosslinking reaction was allowed to proceed for 1 hr at room temp. The resultant LXEI was purified by SEC (MWCO10KD) and buffer exchanged to DDS-water. The efficiency of crosslinking was checked by differential NDN assays using standards that contain PL and PEI. See also, FIG. 19.

CPL Formulation:

CPL was formed by crosslinking Chlorotoxin (C) with PL, where the carboxy-terminal of C was cross-linked to N-terminal or ε-amine on PL. In MES buffer (pH 5.0), 2.5 mg PL (>30 KDa, purified by SEC) was mixed with 800 μg C (at 1:8 molar ratio) in 10 mL total volume and reacted with EDC at 10× molar excess. The reaction was allowed to proceed for 3 hours at room temperature. CPL was purified by SEC (MWCO10K) and buffer was exchanged to 0.1×PBS (pH 7.2). Alternatively Pyridylthiol-activated C was prepared by using SPDP activation in PBSE and purified by SEC. Pyridylthiol activation was tested by Pyridine-2-thione assay detailed above. 2-MEA reaction was used to produce sulfhydryl modified C (SH-C). N-terminal SH-C was purified using SEC via P10 column (GE healthcare). Column purified SH-C was reacted with Pyridylthiol-activated PL at 1:1 molar ratio. The resultant product (designated CPL) was tested by non-denaturing gel electrophoresis and purified by SEC column filtration (MWCO=10 kDa).

PPL Formulation:

methyl-PEG-SH (5000 KDa, JenKem) was purified by SEC suspended in PBSE and reacted with PL-SH at a 2:1 to 4:1 molar ratio for 48 to 72 hours at room temperature. PPL was column purified and buffer exchanged to DDS-water. Efficiency of conjugation and purification was assessed by NDN assay and spectrophotometrically.

PLPL Formulation:

1-Acyl-sn-glycero-3-phospho(2-aminoethanol) (a.k.a., 3-sn-Lysophosphatidylethanolamine), Type I, Sigma; PL, Mw=479 Da, was prepared in chloroform at 5 mg/mL. 150 μg was mixed in 3 mL DMSO and 35.2 μL TEA and further mixed with 2 mg/mL PL in DMSO. Reaction was initiated by 2-iminothiolane at 2× molar excess. After 2.5 hrs, reaction was quenched by flooding reaction vessel with >40-50 mL DMSO. The mixture was purified by SEC column using Diafiltration while stirring in inert gas. 10 mL was mixed with water and lyophilized frozen in a custom device. The lyophilisate was resuspended in DDS-water and filtered through 0.1 μm filter and assessed by differential NDN assay and by spectroscopy. See also, FIG. 18.

TPL Formulation:

In this procedure human holo-Transferrin was activated into Pyridyldithiol-activated forms by reacting with 20 mM solution of N-succinimidyl 3-(2-pyridyldithio) propionate (Mw=312.37; arm length=6.8 Å (SPDP; Thermo Scientific). To do so, Tf was resuspended in PBSE (20 mM Potassium Phosphate, 150 mM NaCl, 1 mM EDTA, 0.02% sodium Azide, pH 7.5). Tf was incubated with SPDP reagent in a 1:4 (Tf:SPDP) molar ratio for 30 minutes at room temperature. At completion, unreacted SPDP were removed and Pyridyldithiol-activated Tf (Tf-PDP) was desalted by size exclusion chromatography (SEC) in a 7 kDa MWCO column (Zeba Spin Columns, Thermo Scientific) as per manufacturer's protocol. The efficiency of SPDP modification was assessed by Pyridine-2-Thione Assay (detailed above). Minimum acceptable mole SPDP per mole Tf was 1.0. To form TPL, Tf-PDP was incubated with PL-SH at 2:1 molar ratio for 48 to 72 hours. TPL was purified by SEC using MWCO-100 KDa column and quantified.

CyPL or RPL Fluorescent moiety Formulation:

1 mg PL was dialyzed against 0.1M NaHCO3 (pH8.3) overnight. The buffer exchanged polymer was reacted with Cy5.5-NHS ester (Mw=592 daltons; Lumiprobe Inc) or Rhodamine-NHS ester (Mw=527.5; Thermo Scientific), at 1:1 molar ratio forming CyPL or RPL, respectively and purified by SEC at MWCO10 KDa and buffer exchanged to DDS-water.

NP Assembly and Nucleic Acid Polymer (NAP) Condensation Producing Final Form Nanoparticles:

During polymeric NP assembly, varying amounts of NP polymeric components (i.e., NP polymer scaffold subunits) were added together forming NP polymeric component (i.e., NP polymer scaffold subunit) skeleton which was used condense nucleic acids and to assemble final form nanoparticles. Nucleic acids, e.g., siRNA, or plasmids expressing GFP as marker, or plasmids expressing therapeutic RNA or proteins or shRNA, or micro-RNA (miRNA), were condensed with specific proportion of NP polymeric components (i.e., NP polymer scaffold subunits) at a nitrogen to phosphate (N:P) charge ratio of 1:32 to 1:67 (i.e., 1 phosphate on the nucleic acid backbone to 32 to 67 nitrogen atoms on the NP polymer scaffold subunits; thus each phosphate on the nucleic acid backbone is ionically captured with 32 to 67 nitrogen atoms on the NP polymer scaffold subunits) and either used directly or purified by SEC to collect nanoparticles of size larger than 100 KDa prior to use. Examples 5-13 below utilized NPs prepared according to the above method.

### Example 5

### Nanoparticle (NP) Preparation for Gene Delivery to Prostate Cells

In this example gene delivery to prostate specific cell line (PC3) was tested using different NP formulations delivering 1.24 μg GFP gene expression plasmids. NP formulations with different levels of AmPL were used.

Materials and Methods:

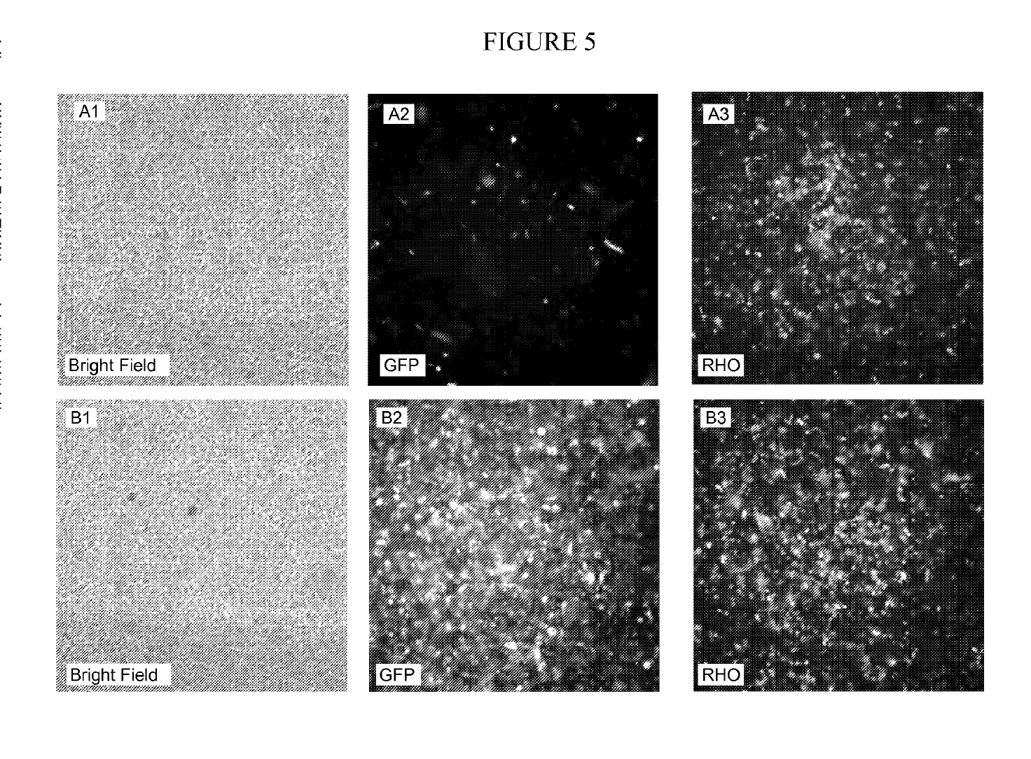

Gene delivery was performed on subconfluent culture of PC3 cells and expression levels were visualized 72-hrs after delivery using bright-filed microscopy FIG. 5 (Panel A1 and B1), green fluorescence imaging (showing GFP expression: Panel A2 and B2) and red fluorescence imaging (showing rhodamine in RPL: Panel A3 and B3).

NP1 (containing: TPL (6.78E+13); LXEI (1.06E+14); PLPL (2.82E+13); AmPL (5.65E+13); RPL (8.48E+12) and PPL (4.24E+13)) and NP2 (containing: TPL (6.78E+13); LXEI (1.06E+14); PLPL (2.82E+13); AmPL (1.06E+14); RPL (8.48E+12) and PPL (4.24E+13)) were condensed with 1.24 μg pGFP plasmid DNA and tested.

Results:

Panel A1-A3 (FIG. 5) shows representative delivery and expression of GFP following delivery with NP1, which contained low AmPL (5.65E+13 molecules) amount versus Panel B1-B3 showing representative delivery and expression of GFP using NP2 which contained high AmPL (1.06E+14 molecules) levels. In comparison to panel A2 (˜5%), panel B2 (˜95%) had several fold higher efficiency of gene delivery, approaching 95% of all live cells.

### Example 6

### Nanoparticle (NP) Driven GFP Delivery and GFP Expression Quantification by Quantitative Real-Time PCR in Primary Human Melanoma Cancers Cells

Materials and Methods:

NPs were tested for delivery and expression of GFP in primary human melanoma cells. NPs were delivered to C81 primary human melanoma cells in triplicate. 72-hours after NP delivery, cells were harvested, and RNA was isolated and purified and used to perform first strand synthesis reaction. The GFP expression was determined by Taqman® (real-time RT-PCR) analysis using probes that were specific for GFP.

The component compositions of the tested nanoparticles are provided below in Tables 2 and 3.

TABLE 2

| | NP3 | NP4 | NP5 | NP6 | NP7 | NP8 |
|---|---|---|---|---|---|---|
| TPL | 1.70E+13 | 1.70E+13 | 1.70E+13 | 1.30E+13 | 8.93E+12 | 4.24E+12 |
| LXEI | 1.06E+14 | 1.06E+14 | 1.06E+14 | 1.06E+14 | 1.06E+14 | 1.06E+14 |
| PLPL | 2.82E+13 | 2.82E+13 | 2.82E+13 | 2.82E+13 | 2.82E+13 | 2.82E+13 |
| AmPL | 1.06E+14 | 1.06E+14 | 1.06E+14 | 1.06E+14 | 1.06E+14 | 1.06E+14 |
| PvPL | 8.48E+12 | 8.48E+12 | 8.48E+12 | 8.48E+12 | 8.48E+12 | 8.48E+12 |
| PPL | 0 | 0 | 0 | 3.14E+13 | 6.28E+13 | 1.26E+14 |
| PEG | 0 | 4.8 μg | 9.6 μg | 0 | 0 | 0 |
| (free) | | | | | | |

TABLE 3

| | NP3 | NP4 | | | |
|---|---|---|---|---|---|
| | PEG | PEG | NP9 | NP10 | NP11 |
| | (0.025%) | (0.05%) | PPL (1x) | PPL (2x) | PPL (3x) |
| TPL | 1.70E+13 | 1.70E+13 | 1.30E+13 | 8.93E+12 | 4.24E+12 |
| LXEI | 1.06E+14 | 1.06E+14 | 1.06E+14 | 1.06E+14 | 1.06E+14 |
| PLPL | 2.82E+13 | 2.82E+13 | 2.82E+13 | 2.82E+13 | 2.82E+13 |
| AmPL | 1.06E+14 | 1.06E+14 | 1.06E+14 | 1.06E+14 | 1.06E+14 |
| PvPL | 8.48E+12 | 8.48E+12 | 8.48E+12 | 8.48E+12 | 8.48E+12 |
| PPL | 0 | 0 | 2.12E+13 | 4.24E+13 | 8.48E+13 |
| PEG | 0 | 4.8 μg | 9.6 μg | 0 | 0 |
| (free) | | | | | |

Results:

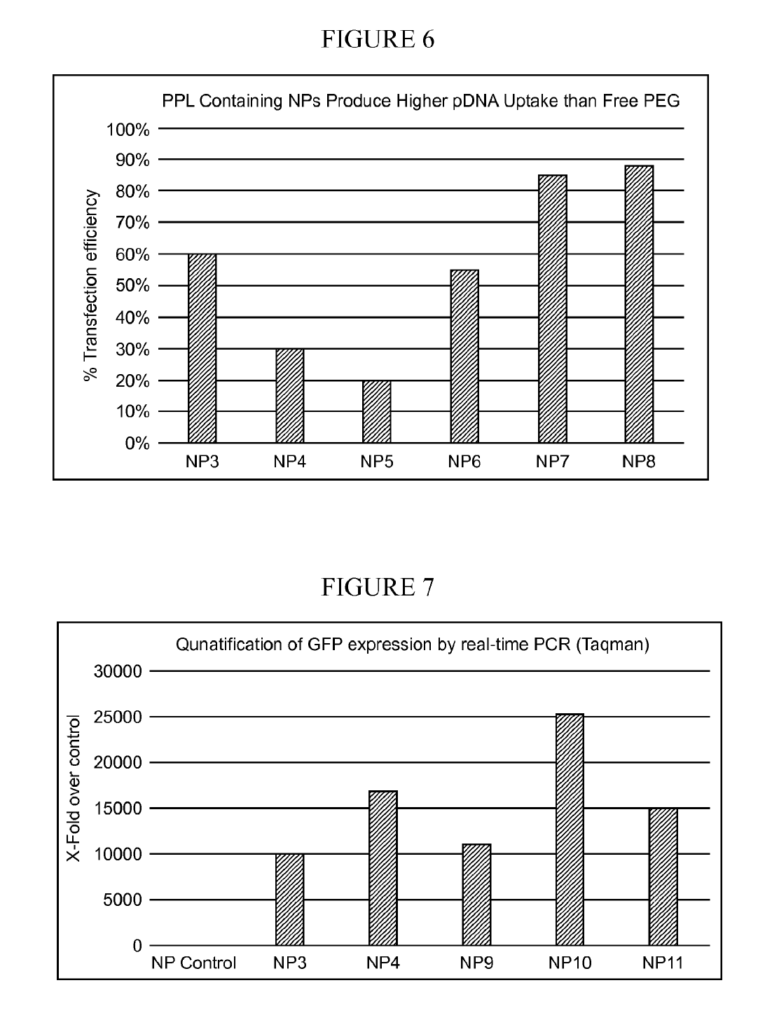

As shown in FIG. 6, PPL containing NPs produced higher pDNA uptake than free PEG, demonstrating the role of PPL in increasing delivery efficiency. In addition, as shown in FIG. 7, the greatest level of expression was obtained after delivery using NP10 with the following proportion of polymers: 8.93E+12 (1%) TPL; 1.06E+14 (31%) LXEI; 2.82E+13 (8%) PLPL; 1.06E+14 (31%) AmPL; 8.48E+12 (3%) RPL; and 4.24E+13 (14%) PPL used to condense 1.24 μg pGFP expression plasmid.

### Example 7

### NP Driven Gene Delivery to Primary Cortex Neurons

Materials and Methods:

Primary rat cortex neurons were grown on poly-d-lysine. NP12 containing 8.93E+12 (1%) TPL; 1.06E+14 (31%) LXEI; 2.82E+13 (8%) PLPL; 1.06E+14 (31%) AmPL; 8.48E+12 (3%) RPL; and 8.48E+13 (14%) PPL were used to condense 1.24 μg pGFP expression plasmids. The primary rat cortex neurons were exposed to fully formulated NP for 4 hours and observed by fluorescence microscopy after 3-days.

Results:

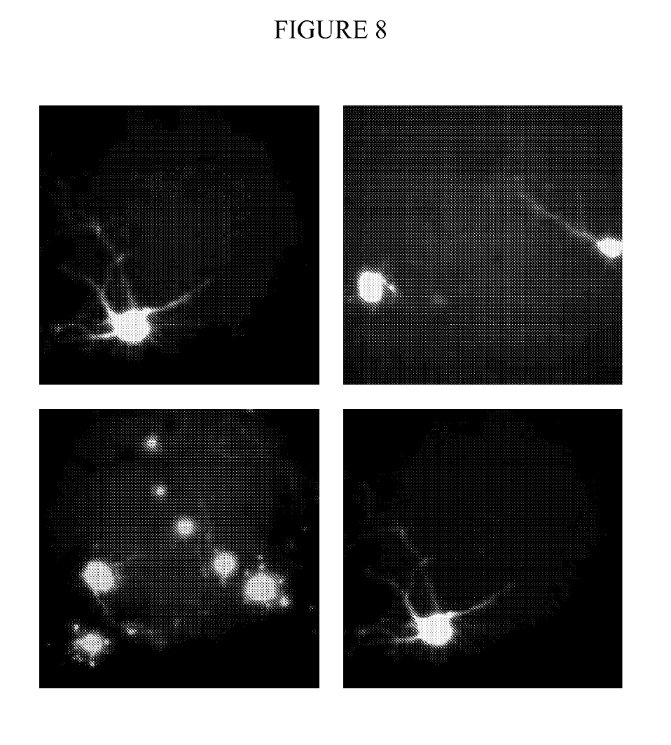

As shown in FIG. 8, the NP containing the above proportion of polymers demonstrated specific uptake and expression of GFP in primary rat cortex neurons.

### Example 8

### Therapeutic NP-siRNA Delivery Targeting BPTF Gene Expression in Primary Human Melanoma Cells

Bromodomain PHD Finger Transcription Factor (BPTF) is highly up-regulated in human melanomas and is required for melanoma metastasis. NPs containing BPTF specific siRNA were prepared and tested for their ability to down-regulate BPTF/FALZ transcripts as follows.

Materials and Methods:

NP10 was prepared with 8.93E+12 (3%) TPL; 1.06E+14 (35%) LXEI; 2.82E+13 (9%) PLPL, 1.06E+14 (35%) AmPL; 8.48E+12 (3%) RPL; and 4.24E+13 (14%) PPL, and used to condense 1.24 μg BPTF siRNA. These NP were delivered to C81 primary human melanoma cells in triplicate. 72-hours after NP deliver, cells were harvested, and RNA was isolated and purified and used to perform first strand synthesis reaction. BPTF expression was determined by Taqman® analysis, in triplicate, using probes that were specific for human BPTF, where HPRT specific Taqman® probes were used as internal control and NP carrying non-specific siRNA were used as cell specific control. The BPTF siRNA sequence used in this example is provided below for reference.

TABLE 4

| | Sense (5′-3′) | Antisense (5′-3′) |
|---|---|---|
| BPTF | rCrArUrArArUrArUrCrGrUrCr | rArGrArGrUrArCrArArArGrArCrGr |
| siRNA1 | UrUrUrGrUrArCrUrCrUraaa | ArUrArUrUrArUrGraaa |

Results:

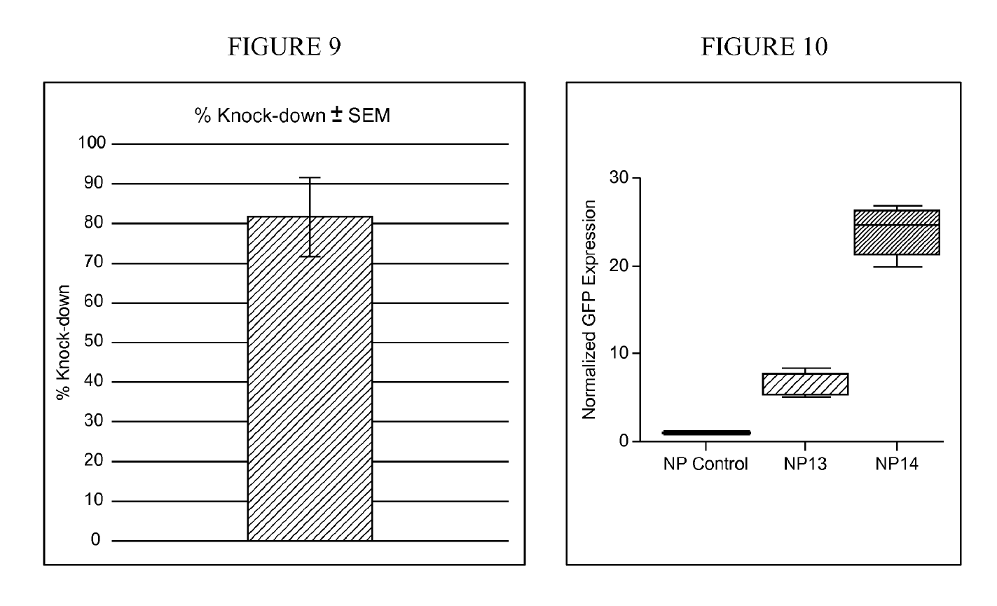

As shown in FIG. 9 in primary human melanoma cells treated with NP10, carrying BPTF specific siRNA, BPTF expression was specifically Knocked-down by 81.66±1.51%.

### Example 9

### CPL Containing NP Based Gene Delivery Via i.v. Injection Produces Significantly Enhanced Delivery and Gene Expression in the Brain

Materials and Methods:

Formulated NP condensed with NP plasmids were injected into mice via tail vein (i.v. injections). Each injection was composed of 3× of either: NP13) 8.93E+12 (3%) TPL; 1.06E+14 (32%) LXEI; 2.82E+13 (9%) PLPL; 1.06E+14 (32%) AmPL; 1.87E+13 (6%) CyPL; and 6.33E+13 (19%) PPL used to condense 1.24 μg pGFP expression plasmid, or NP14) 8.93E+12 (3%) TPL; 1.06E+14 (32%) LXEI; 2.82E+13 (9%) PLPL; 8.93E+13 (27%) AmPL; 1.87E+13 (6%) CyPL; 6.33E+13 (19%) PPL, and 1.70E+13 (5%) CPL used to condense 1.24 μg pGFP expression plasmid, or 3) control NP condensed with non-GFP plasmid as mock delivery control. Three injections were given 6 days apart. Four days after the final injection, brain tissues were harvested, brain RNA isolated and subjected to Taqman® real-time PCR analysis.

The component compositions of the tested nanoparticles are provided below in Table 5.

TABLE 5

| | NP13 (3x) | NP14 (3x) |
|---|---|---|
| TPL | 8.93E+12 | 8.93E+12 |
| LXEI | 1.06E+14 | 1.06E+14 |
| PLPL | 2.82E+13 | 2.82E+13 |
| AmPL | 1.06E+14 | 8.93E+13 |
| CyPL | 1.87E+13 | 1.87E+13 |
| PPL | 6.33E+13 | 6.33E+13 |
| CPL | | 1.70E+13 |
| pGFP | 1.24 μg | 1.24 μg |

Results:

The results of the above in vivo experiment are shown in FIG. 10 and demonstrate that CPL containing NPs significantly enhanced NP gene delivery and expression in the brain after i.v. injections.

### Example 10

### Organ Specific Gene Delivery and Expression Following Intra-Venous Delivery In Vivo

The aim of this study was to quantitatively determine organ specific gene delivery and gene expression following tail vein injection in mice.

Materials and Methods:

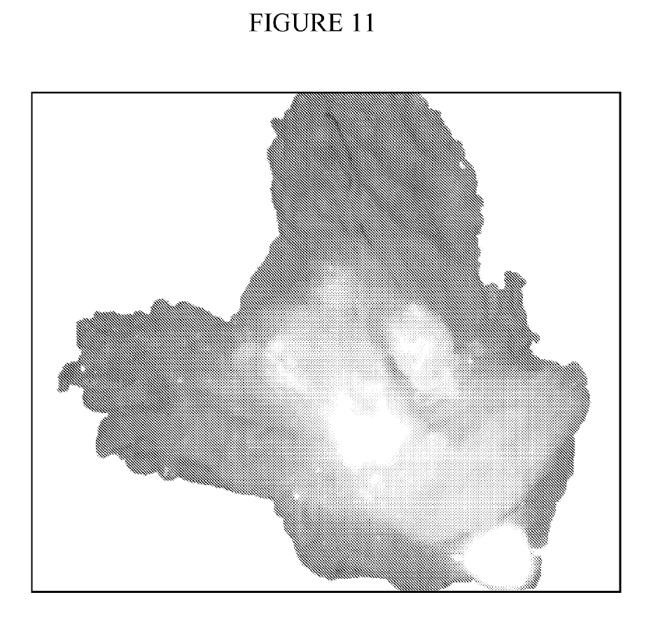

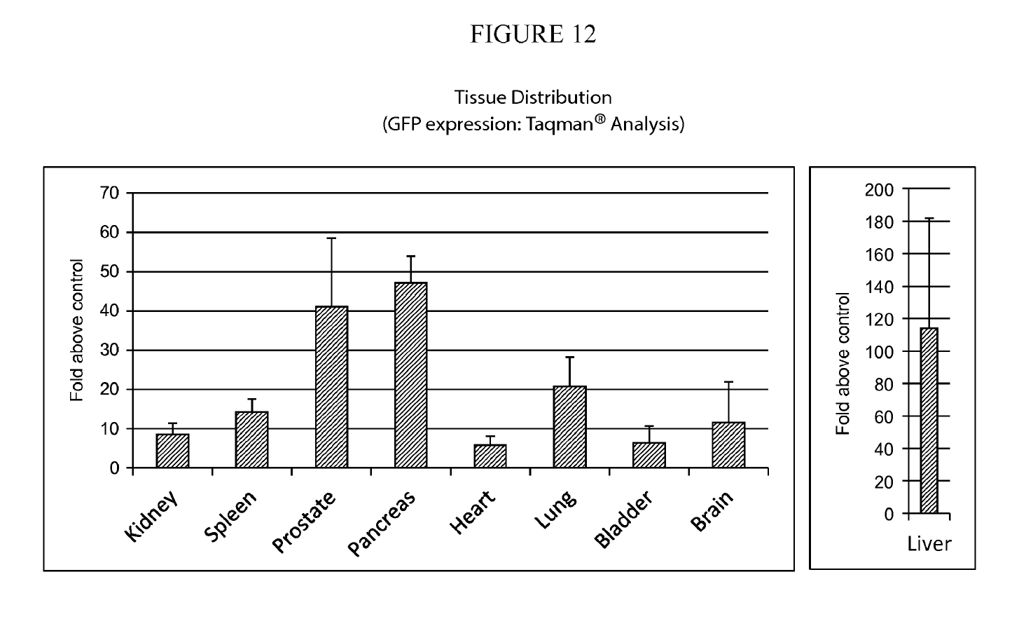

3×NP15 containing 8.93E+12 (3%) TPL; 1.06E+14 (31%) LXEI; 2.82E+13 (8%) PLPL; 1.06E+14 (31%) AmPL; 8.48E+12 (3%) CyPL; and 6.33E+13 (19%) PPL were condensed with 1.24 μg pGFP, delivering 3.72 μg pGFP in total volume of 200 μL per injection. Mature nude mice were given 3 injections via tail vein every 6 days condensed with either GFP plasmid or a reference non-GFP plasmid. Four days following the final injection, mice were euthanized per IACUC approved procedures. The following organs were collected to be quick-frozen in liquid nitrogen for RNA analysis: brain, kidneys, liver, prostate, bladder, spleen, lungs and heart. Prior to isolation of mRNA and Taqman® analysis, prostate was imaged for GFP expression (FIG. 11) while other organs being too dense to be imaged for GFP—were immediately processed for quantitative real-time PCR study using Taqman® analysis. Following imaging, prostate and bladder were also processed for Taqman analysis. Control reference RNA were used as mock delivery controls, while RAB14 gene expression was used as an internal control for quantitation. GFP expression was calculated as RNA was isolated via Thermo Scientific GeneJET RNA Purification Kit. The RNA was then reverse transcribed using BioRad iScript™ cDNA Synthesis Kit. Next, Taqman probe/primer sets specific for GFP and the loading control, Rab14 were run on an Applied Biosystems 7500 Real Time PCR machine to assess Ct values for both GFP and Rab14 probe/primer sets. ΔΔCT calculations were then performed utilizing the loading control Rab14 as an internal control. Baseline levels were defined as the nonspecific background probe/primer binding in non-GFP containing nanoparticle treated mouse tissue (FIG. 12).

Results:

Four days after the last of 3 injections, steady state distribution of gene expression following i.v. injection with NP condensed with 3.72 μg plasmid DNA driving quantifiable expression of GFP marker gene was observed in liver, kidneys, lungs, brain, heart, spleen, bladder, prostate and pancreas, and, ranged from over 5-fold above baseline in the heart to over 100-fold above baseline levels in the liver (FIG. 12).

### Example 11

### Therapeutic microRNA Delivery to Brain In Vivo for Glioblastoma Multiforme

Micro-RNAs (miRNAs) regulate gene expression by promoting mRNA degradation or inhibiting translation of critical regulatory genes. In human GBMs, several miRNAs have been described to modulate both oncogenic as well as tumor suppressor signaling networks, and thus represent attractive therapeutic targets.

In this example, NPs were developed for targeted delivery of mir-34a and mir-128 and their effect on downstream targets was determined. The targeted miRNA-NP were injected into mice bearing primary human GBM tumors by i.v. injection to test the therapeutic benefits of overexpressing these miRNAs in a mouse model of disease.

Rationale for Over-Expressing Mir-34a:

Human-micro-RNA-34a (mir-34a) is significantly down-regulated in aggressive GBM. (See, e.g., Moller H G et al. Mol Neurobiol. 2013; 47:131-144). It is a required component of a regulatory network controlling: 1) Viability, 2) Proliferation, 3) invasiveness, 4) in vivo tumor growth and 5) dedifferentiation through its action on critical regulatory transcriptomes. (See, e.g., Li Y. et al. Cancer Res. 2009; 69:7569-7576; Guessous F. et al. Cell Cycle. 2010; 9:1031-1036; Silber J. et al. PLoS One. 2012; 7:e33844; and Vo D. T. et al. RNA Biol. 2011; 8:817-828). Mir-34a expression is regulated by the tumor suppressor p53, which, in turn, regulates expression and activity levels of PDGFRA and TGFβ-Smad4-ID1 signaling pathways, among others. Mir-34a itself acts as tumor suppressor in a transgenic mouse model of GBM, by directly inhibiting PDGFRA and the Smad4-Id1 signaling pathways. (See, e.g., Misso G. et al. Mol Ther Nucleic Acids. 2014; 3:e194). It has been previously demonstrated that targeted inhibition of ID1 gene expression confers a survival benefit to mice bearing human glioma xenografts. (See, e.g., Soroceanu L. et al. Cancer Res. 2013; 73:1559-1569). Therefore, targeted over-expression of mir-34a is predicted to have a tumor inhibitory effect in GBM, especially in those tumors driven by PDGFRA alterations or ID1 overexpression.

Rationale for Over-Expressing Mir-128:

Human-micro-RNA-128 (mir-128) is highly expressed in neurons, but at critically reduced levels in glioma tissue. (See, e.g., Moller H G et al. Mol Neurobiol. 2013; 47:131-144). Primary targets of mir-128 are the polycomb group proteins Bmi1 and Suz12, (Peruzzi P. et al. Neuro Oncol. 2013; 15:1212-1224) which play an important role in maintaining the undifferentiated status of normal and cancerous neural stem cells. Dong Q. et al. PLoS One. 2014; 9:e98651). Published studies demonstrated that by down-regulating Bmi1, mir-128 directly inhibited GSC self-renewal and promoted differentiation toward the neuronal lineage. (See. e.g., Papagiannakopoulos T. et al. Oncogene. 2012; 31:1884-1895). Mir-128 also targets and inhibits activity of several oncogenic cellular kinases, including EGFR (amplified in ˜50% of human GBMs), and p-AKT, which promote cancer cell proliferation.

Mir-34a and Mir-128 Sequences:

The mir-34a and mir-128 sequences used in this example are provided below for reference:

1. hsa-miR-34a-5p MI00002268 mature sequence
UGGCAGUGUCUUAGCUGGUUGU
which could also be used as the following stem-
loop sequence: hsa-mir-34a MI0000268
GGCCAGCUGUGAGUGUUUCUUUGGCAGUGUCUUAGCUGGUUGUUGUGA
GCAAUAGUAAGGAAGCAAUCAGCAAGUAUACUGCCCUAGAAGUGCUGC
ACGUUGUGGGGCCC
2. hsa-miR-128-2-5p MI0000727 mature sequence
GGGGGCCGAUACACUGUACGAGA
which could also be used as the following stem-
loop sequence: hsa-mir-128-2 MI0000727
UGUGCAGUGGGAAGGGGGGCCGAUACACUGUACGAGAGUGAGUAGCAG
GUCUCACAGUGAACCGGUCUCUUUCCCUACUGUGUC

Design, Testing and Validation of Targeted NP:

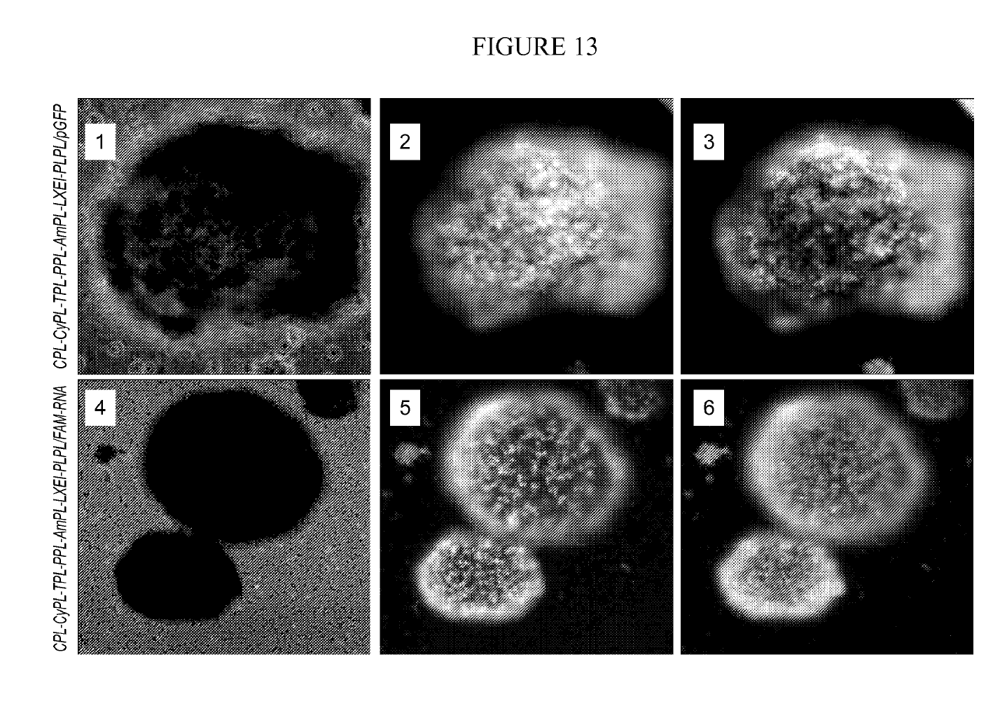

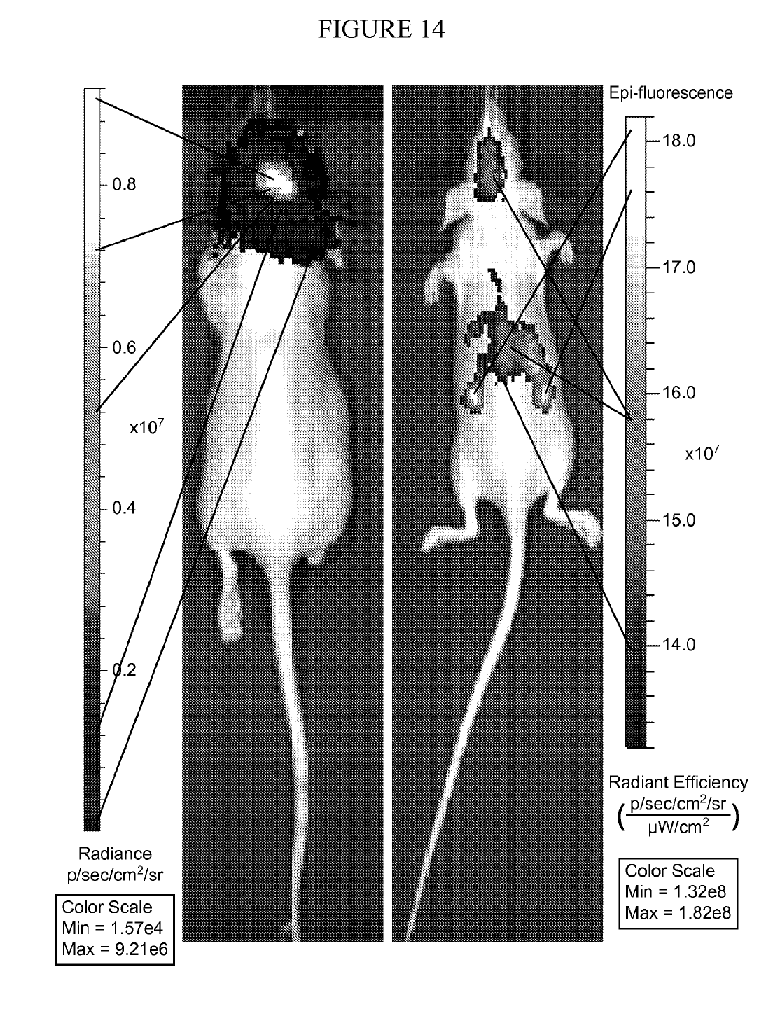

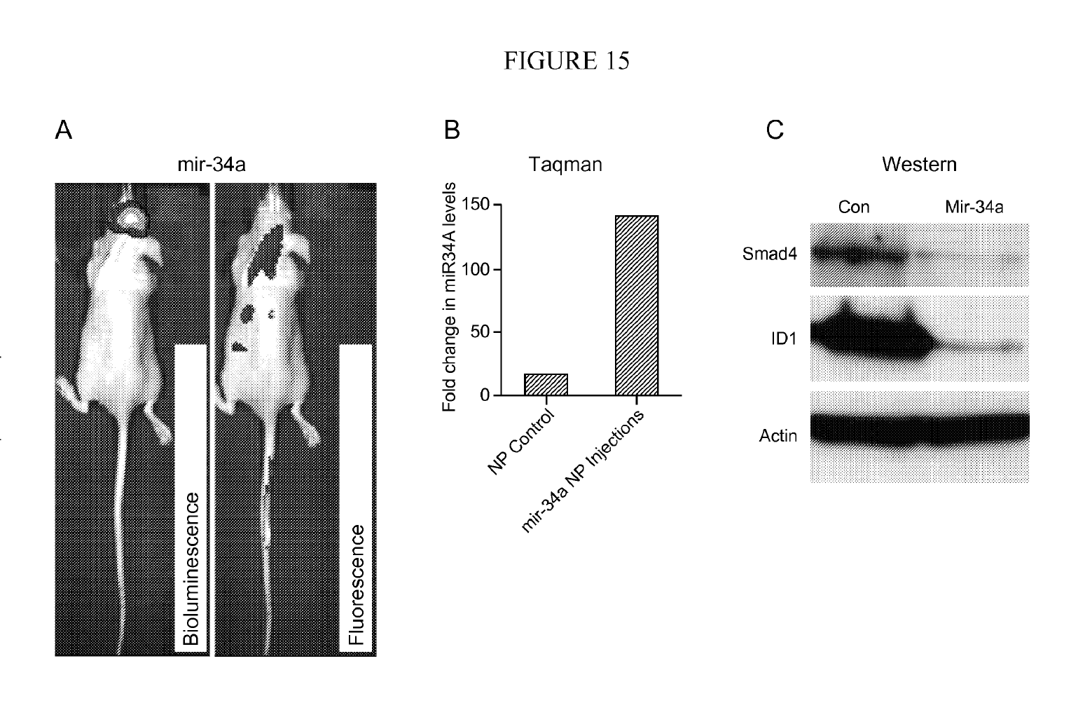

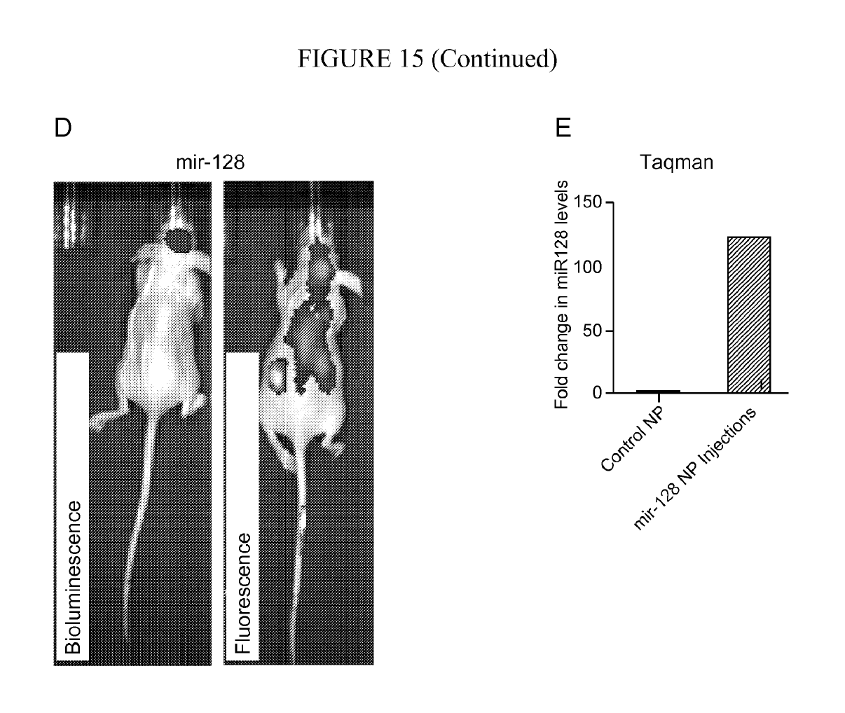

Several NP with different attributes and conjugate stoichiometries were designed and tested. These included conjugated tissue targeting surface markers such as ClTx (CPL) and Tf (TPL), and covalently attached PEG (PPL) for in vivo stability. Stoichiometric ratios of surface moieties and scaffolds designed for delivery of GFP plasmid DNA and FAM labeled small RNA were tested for in vitro delivery into primary human GBM neurospheres (FIG. 13) and in vivo in mice bearing primary human GBM tumors (FIGS. 14 and 15).

The final form NP used in these tests were produced by assembling specific ratios of component conjugated polymer and formulated copolymers, these components were: 1) PL polymeric scaffold covalently bonded to PEG (PPL), providing NP the ability to evade host immune mechanisms, 2) PL conjugated to N-terminus of Tf ligand (TPL) for passage through the BBB, 3) PL with surface attached ClTx (CPL) to specifically target cells of neuroectodermal lineage, especially GBM cells, 4) PL with bound amphiphile Am1 (AmPL), and copolymers: 5) LXEI and 6) PLPL, where AmPL, LXEI and PLPL were included to facilitate endosomal escape enhancing efficiency. An additional feature of the tested NPs is surface available peptide bonds which allow easy degradation and kidney clearance of the NP degradation products, thereby reducing systemic toxicity. For intracellular tracking or for tissue localization, covalently attached far-red fluorescent dye (Cy5.5 (CyPL) for in vivo localization or Rhodamine (RPL) for in vitro localization) was also included. NP prepared from 10⁴ nucleic acid polymer (NAP) condensed with NP assembled from component polymers (NP polymer scaffold subunits: CPL, RPL, TPL, PPL, AmPL, LXEI and PLPL) forming CPL-RPL-TPL-PPL-AmPL-LXEI-PLPL/pGFP (NP16) or CPL-RPL-TPL-PPL-AmPL-LXEI-PLPL/FAM-RNA (NP17) demonstrated minimum cellular toxicity and highest expression of eGFP or nuclear delivery of FAM labeled small nuclear RNA in cultured GBM cells grown as neurospheres (FIG. 13). Therefore, this NP formulation was chosen for in vivo delivery into human primary GBM mouse model by tail vein injection. The component composition for NP16 and NP17 is provided below.

TABLE 6

| NP | NP16: CPL-RPL-TPL-PPL-AmPL- | NP17: CPL-RPL-TPL-PPL-AmPL-LXEI- |
|---|---|---|
| compositions | LXEI-PLPL/pGFP | PLPL/FAM-RNA |
| TPL | 8.93E+12 | 8.93E+12 |
| LXEI | 1.06E+14 | 1.06E+14 |
| AmPL | 2.82E+13 | 2.82E+13 |
| PLPL | 8.93E+13 | 8.93E+13 |
| RPL | 8.48E+12 | 8.48E+12 |
| PPL | 8.48E+13 | 8.48E+13 |
| CPL | 1.70E+13 | 1.70E+13 |
| NAP: Nucleic | pGFP: plasmid w/GFP | FAM labeled small RNA |
| Acid Polymer | (1.24 μg) | (1.24 μg) |

Mice implanted with primary human GBM neurospheres forming a PDX model of human GBM were i.v. injected with 3×NP16 delivering 3.72 μg GFP plasmid every 72 hrs. These mice were imaged for bioluminescence following luciferin treatment to visualize tumor size (FIG. 14, left image) of mice bearing human patient derived tumor in the intracranial region. The same mice were imaged for NP delivery by far-red Cy5.5 dye imaging (FIG. 14, right image). As shown in right image, NP16 efficiently crossed the BBB, reaching the tumor in the intracranial region.

In vivo delivery capabilities of the NP described above were then tested. Specifically, NP16 carrying mir-34a in place of pGFP (CPL-CyPL-TPL-PPL-AmPL-LXEI-PLPL/mir-34a) and NP16 carrying mir-128 in place of pGFP (CPL-CyPL-TPL-PPL-AmPL-LXEI-PLPL/mir-128) were tested. The results (FIG. 15) demonstrated that stabilized and targeted NP delivered systemically, via i.v. injection, delivered miRNA to GBM intracranially in sufficient quantity to specifically overexpress mir-34a and mir-128 within the tumor tissue and down-regulate downstream targets (FIG. 15). 48 hours following systemic administration via tail vein injection, fluorescently labeled NP encoding mir-34a and mir-128 were found primarily within the brain tumor tissue (derived from GSC3832), with additional distribution in the clearing organs (liver, kidney, spleen). One week following two sequential i.v. administrations of NP delivering therapeutic miRNAs, brain tissue was homogenized and used for Taqman and Western blot measurements (FIG. 15). Multiple injections of the NP formulation did not cause any overt toxicity during the entire study period. A shown in FIG. 15, mir-34a and mir-128 where both up-regulated over 100-fold in tumor tissue. Overexpression of mir-34a in tumor tissue led to the corresponding robust down-regulation of Smad4 and ID1, two proteins that critically promote tumor progression (FIG. 15, Panel C).

### Example 12

### ACTX-01a Pilot Study

### Therapeutic Targeting of Human Primary Patient Derived Glioblastoma Xenograft in Mice (GBM PDX Model) Achieved Tumor Shrinkage and Target Knock-Down within 1-Week of Treatment with CD44 siRNA NP (ACTX-01a)

GBM tumors are extremely difficult to treat, primarily because they contain glioblastoma stem cells (GSC) which promote tumor resistance to therapies. Most aggressive GSCs can switch and adapt their proliferative pathways to promote cancer recurrence. These recurrence pathways make GBM an aggressive and deadly disease. New NPs were formulated condensed with siRNA against CD44 (ACTX-01a) to down-regulate CD44 gene expression, a gene product critical for stem cell regeneration and proliferation. The siRNA sequence used in this example is provided below for reference.

TABLE 7

| | Sense (5′-3′) | Antisense (5′-3′) |
|---|---|---|
| CD44 | rGrCrGrCrArGrArUrCrGrArUrU | rUrArUrUrCrArArArUrCrGrAr |
| siRNA | rUrGrArArUrAtt | UrCrUrGrCrGrCca |
| (ACTX- | | |
| 01a) | | |

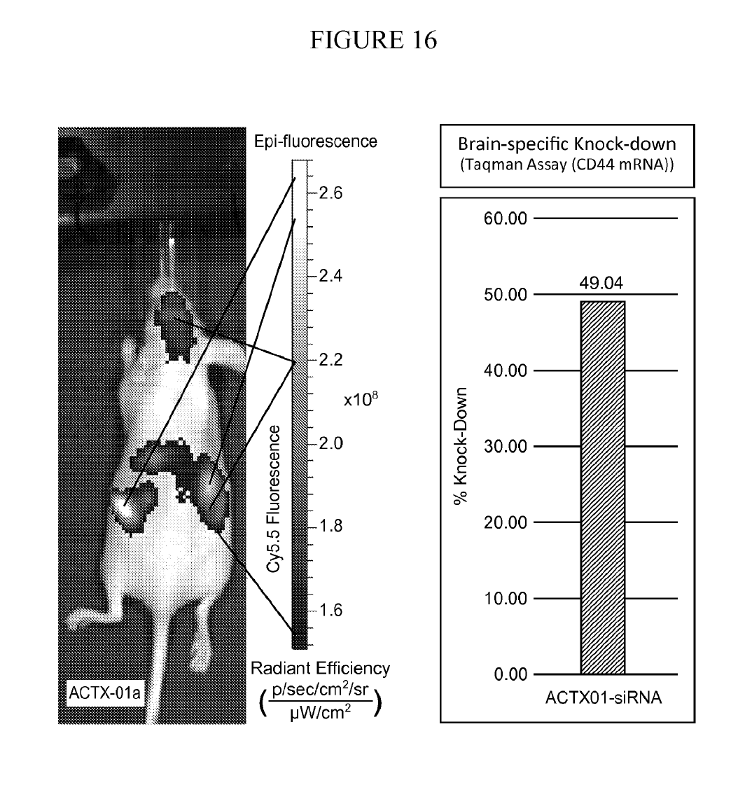

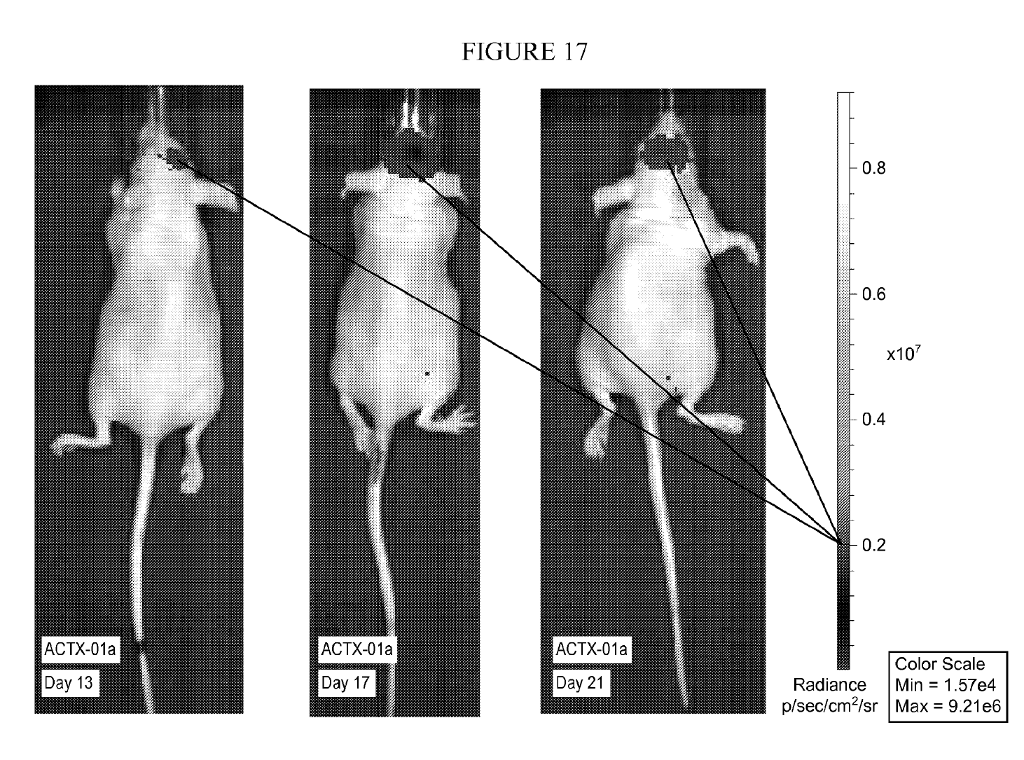

An aggressive luciferase positive GSC3832 tumor cell line, which recapitulates most aggressive GBM in patients was used in these in vivo studies. In GSC38332 neurospheres, the ACTX-01a specifically downregulated CD44 mRNA and protein. Tail vein injection (iv) delivery of ACTX-01a successfully delivered CD44 siRNA to the brain, where it could specifically down-regulate CD44 expression by 49% (FIG. 16). Pilot studies where therefore performed, in a PDX mouse model of GBM, in which ACTX-01a was delivered (3×NP14 containing 8.93E+12 (3%) TPL; 1.06E+14 (32%) LXEI; 2.82E+13 (9%) PLPL; 8.93E+13 (27%) AmPL; 1.87E+13 (6%) CyPL; 6.33E+13 (19%) PPL, and 1.70E+13 (5%) CPL used to condense 1.24 μg CD44 siRNA) via tail vein injection on day 13 and day 17 and observed changes in tumor size up to day 21. Intravenous injection with ACTX-01a noticeably reduced GBM tumor size in the mouse brain (FIG. 17).

### Example 13

### Preclinical Efficacy of ACTX-01a (CD44 siRNA/NP) and ACTX-01b (Sox2 siRNA/NP) in PDX Mouse Model

Materials and Methods:

In order to assess efficacy of NP gene therapy, a large preclinical trial was initiated. Primary human GBM stem cells (GSC3832) were intracranially implanted into juvenile (8-wk. old) nude mice. Fourteen days after implantation, tumors were imaged for luminescence and randomized into three groups of eight each: 1) control group (n=8) receiving i.v. injection of NP condensed with random non-specific siRNA (ACTX-00 group), 2) ACTX-01a group (n=8) receiving i.v. injection of NP condensed with CD44 siRNA and 3) ACTX-01b group (n=8) receiving i.v. injection of NP condensed with Sox2 siRNA (Note: ACTX-01b has the same NP composition as ACTX-01a with the exception of the replacement of CD44 siRNA with Sox2 siRNA). The siRNA sequences used in this example is provided below for reference.

TABLE 8

| | Sense (5′-3′) | Antisense (5′-3′) |
|---|---|---|
| CD44 | rGrCrGrCrArGrArUrCrGrArUrUr | rUrArUrUrCrArArArUrCrGrArUrCr |
| siRNA | UrGrArArUrAtt | UrGrCrGrCca |
| (ACTX- | | |
| 01a) | | |
| Sox2 | rUrGrGrUrCrArUrGrGrArGrUrUr | rCrArGrUrArCrArArCrUrCrCrArUr |
| (ACTX- | CrUrArCrUrGca | GrArCrCrAcg |
| 01b) | | |
| siRNA1 | | |
| Sox2 | rUrUrCrArUrGrUrArGrGrUrCrUr | rGrCrUrCrGrCrArGrArCrCrTrArCr |
| (ACTX- | GrCrGrArGrCtg | ArTrGrArAcg |
| 01b) | | |
| siRNA2 | | |
| Sox2 | rUrArCrUrUrArUrCrCrUrUrCrUr | rUrCrArUrGrArArGrArArGrGrArUr |
| (ACTX- | UrCrArUrGrAgc | ArArGrUrAca |
| 01b) | | |
| siRNA3 | | |
| Sox2 | rArArCrCrCrArUrGrGrArGrCrCr | rGrCrUrCrUrUrGrGrCrUrCrCrArUr |
| (ACTX- | ArArGrArGrCca | GrGrGrUrUcg |
| 01b) | | |
| siRNA4 | | |

Tail vein NP injections (200 μL) were performed twice every week. To monitor health, mice in each treatment group were weighed 3 times per week and were observed daily. Tumor size was monitored by luminescence image analysis in a Caliper IVIS in vivo imaging system.

Tumor size was quantified and compared to the control (ACTX-00) group using Caliper on board software analysis system (Xenogen, Inc).

Results:

As detailed in Table 9, the trajectory of tumor growth in ACTX-01 and ACTX-01b groups was significantly reduced starting at day-7 after the first i.v. injection. At this time, tumor sizes in the ACTX-01a and ACTX-01b groups were ˜3 times smaller than in the control ACTX-00 group. The average tumor size reduction became more pronounced, increasing to over 6-fold in the ACTX-01a group by day 10. Overall, through the entire treatment period (7 to 21 days) the average fold reduction in ACTX-01a group was 3.12±0.85, and 3.40±0.89 in the ACTX-01b group. Though the tumor sizes eventually were equalized at day-21, the end of the study period, this study demonstrated that the targeted NP successfully delivered siRNAs to the brain tumors which were effective in noticeably reducing tumor sizes in vivo.

TABLE 9
Average Radiance (Tumor Size)

| Days after first injection: | 0 | 7 | 10 | 14 | 17 | 21 |
|---|---|---|---|---|---|---|
| ACTX-00 (control grp) | 1.90E+03 | 8.97E+04 | 2.63E+05 | 9.18E+05 | 1.99E+06 | 4.76E+06 |
| ACTX-01a (NP/siCD44 | 2.57E+03 | 3.24E+04 | 4.20E+04 | 2.95E+05 | 8.14E+05 | 4.65E+06 |
| grp) | | | | | | |
| ACTX-01b (NP/siSox2 | 2.77E+03 | 1.26E+04 | 7.14E+04 | 4.70E+05 | 8.93E+05 | 2.34E+06 |
| grp) | | | | | | |

| | | |
|---|---|---|
| ACTX-01a (avg. | — | Fold Tumor size reduction (over control) = 3.12 ± 0.85 |
| reduction) | | |
| ACTX-01b (avg. | — | Fold Tumor size reduction (over control) = 3.40 ± 0.89 |
| reduction) | | |

What is claimed is:

1. A polymeric nanoparticle comprising an aggregate of nucleic acids and polycationic polymer scaffolds, wherein the aggregate comprises:
a blood brain barrier (BBB) transport moiety covalently bound to a first polycationic polymer scaffold;
an amphiphilic peptide and/or target binding moiety covalently bound to a second polycationic polymer scaffold;
a hydrophilic polymer covalently bound to a third polycationic polymer scaffold;
an amphiphilic peptide covalently bound to a fourth polycationic polymer scaffold;
a fifth polycationic polymer scaffold comprising a copolymer of a polycationic polymer and a hydrophobic polymer (PLPL);
a sixth polycationic polymer scaffold comprising a copolymer of a polycationic polymer and a polyethylenimine (LXEI); and
a nucleic acid bound by ionic-charge interactions to the polycationic polymer scaffolds.

2. The polymeric nanoparticle of claim 1, wherein two or more of the first through sixth polycationic polymer scaffolds are the same.

3. The polymeric nanoparticle of claim 1, wherein two or more of the first through the sixth polycationic polymer scaffolds are distinct polymers.

4. The polymeric nanoparticle of claim 3, wherein each of the first through the sixth polycationic polymer scaffolds is a distinct polycationic polymer scaffold.

5. The polymeric nanoparticle of any one of claims 2-4, wherein the BBB transport moiety covalently bound to a first polycationic polymer scaffold is a polycationic polymer-bound Transferrin (TPL).

6. The polymeric nanoparticle of any one of claims 2-5, wherein the amphiphilic peptide and/or target binding moiety covalently bound to a second polycationic polymer scaffold is a polycationic polymer-bound chlorotoxin (CPL).

7. The polymeric nanoparticle of any one of claims 2-6, wherein the hydrophilic polymer covalently bound to a third polycationic polymer scaffold is a polycationic polymer-bound PEG (PPL).

8. The polymeric nanoparticle of any one of claims 2-7, wherein the amphiphilic peptide covalently bound to a fourth polycationic polymer scaffold is a polycationic polymer-bound Am1peptide (AmPL).

9. The polymeric nanoparticle of any one of claims 2-8, wherein the fifth polycationic polymer scaffold comprising a copolymer of a polycationic polymer and a hydrophobic polymer (PLPL) is a poly-lysine conjugated to lyso-phosphatidylethanolamine.

10. The polymeric nanoparticle of any one of claims 2-9, comprising a polycationic polymer scaffold comprising a detectable label.

11. The polymeric nanoparticle of claim 10, wherein the detectable label is a fluorescent label.

12. The polymeric nanoparticle of claim 11, wherein the polycationic polymer scaffold comprising a detectable label is a polycationic polymer-bound Cy5.5 fluorescent label (CyPL) or polycationic polymer-bound rhodamine (RPL).

13. The polymeric nanoparticle of any one of claims 2-12, wherein each of the first through the sixth polycationic polymer scaffolds is a distinct polycationic polymer scaffold, and wherein 2% to 12% of the total number of polycationic polymer scaffolds which make up the nanoparticle are polycationic polymer scaffolds in which the BBB transport moiety is covalently bound to the first polycationic polymer scaffold.

14. The polymeric nanoparticle of any one of claims 2-13, wherein each of the first through the sixth polycationic polymer scaffolds is a distinct polycationic polymer scaffold, and wherein 3% to 10% of the total number of polycationic polymer scaffolds which make up the nanoparticle are polycationic polymer scaffolds in which the amphiphilic peptide and/or target binding moiety is covalently bound to the second polycationic polymer scaffold.

15. The polymeric nanoparticle of any one of claims 2-14, wherein each of the first through the sixth polycationic polymer scaffolds is a distinct polycationic polymer scaffold, and wherein 14% to 35% of the total number of polycationic polymer scaffolds which make up the nanoparticle are polycationic polymer scaffolds in which the hydrophilic polymer is covalently bound to the third polycationic polymer scaffold.

16. The polymeric nanoparticle of any one of claims 2-15, wherein each of the first through the sixth polycationic polymer scaffolds is a distinct polycationic polymer scaffold, and wherein 25% to 35% of the total number of polycationic polymer scaffolds which make up the nanoparticle are polycationic polymer scaffolds in which the amphiphilic peptide is covalently bound to the fourth polycationic polymer scaffold.

17. The polymeric nanoparticle of any one of claims 2-16, wherein each of the first through the sixth polycationic polymer scaffolds is a distinct polycationic polymer scaffold, and wherein <1% to 11% of the total number of polycationic polymer scaffolds which make up the nanoparticle are the fifth polycationic polymer scaffold comprising a copolymer of a polycationic polymer and a hydrophobic polymer (PLPL).

18. The polymeric nanoparticle of any one of claims 2-17, wherein each of the first through the sixth polycationic polymer scaffolds is a distinct polycationic polymer scaffold, and wherein 25% to 35% of the total number of polycationic polymer scaffolds which make up the nanoparticle are the sixth polycationic polymer scaffold comprising a copolymer of a polycationic polymer and a polyethylenimine (LXEI).

19. The polymeric nanoparticle of any one of claims 2-18, wherein each of the first through the sixth polycationic polymer scaffolds is a distinct polycationic polymer scaffold, wherein the nanoparticle comprises a polycationic polymer scaffold comprising a detectable label, and wherein 1% to 3% of the total number of polycationic polymer scaffolds which make up the nanoparticle are the polycationic polymer scaffold comprising the detectable label.

20. The polymeric nanoparticle of any one of claims 2-19, wherein one or more of the polycationic polymer scaffolds comprises one or more of poly-lysine, poly-arginine, poly-glutamine, poly-amine, poly(diallydimethylammonium chloride) (pDADMAC), and chitosan.

21. The polymeric nanoparticle of any one of claims 2-20, wherein the nucleic acid comprises DNA.

22. The polymeric nanoparticle of claim 21, wherein the DNA encodes an interfering RNA.

23. The polymeric nanoparticle of claim 22, wherein the interfering RNA comprises a short-hairpin RNA (shRNA).

24. The polymeric nanoparticle of claim 21 or 22, wherein the interfering RNA comprises a sequence complementary to a portion of a gene transcript for CD-44, CD-44 V6, Sox2, Id-1, Id-3, c-Met, or NCOA3.

25. The polymeric nanoparticle of any one of claims 2-20, wherein the nucleic acid comprises RNA.

26. The polymeric nanoparticle of claim 25, wherein the RNA comprises interfering RNA.

27. The polymeric nanoparticle of claim 26, wherein the interfering RNA comprises a small interfering RNA (siRNA).

28. The polymeric nanoparticle of claim 26, wherein the interfering RNA comprises a shRNA.

29. The polymeric nanoparticle of claim 26, wherein the interfering RNA comprises a micro-RNA (miRNA).

30. The polymeric nanoparticle of any one of claims 26-29, wherein the interfering RNA comprises a sequence complementary to a portion of a gene transcript for CD-44, CD-44 V6, Sox2, Id-1, Id-3, c-Met, or NCOA3.

31. A method of reducing the expression of a target protein in a cell, the method comprising contacting the cell with a polymeric nanoparticle according to any one of claims 1-20, wherein the nucleic acid is an interfering RNA or a DNA encoding an interfering RNA bound by ionic-charge interactions to the polycationic polymer scaffolds, wherein the interfering RNA comprises a sequence complementary to a portion of a gene transcript for the target protein, and whereby expression of the target protein is reduced relative to expression of the target protein in the absence of the contacting.

32. A method of treating Glioblastoma Multiforme (GBM) in a subject, the method comprising: administering a therapeutically effective amount of a formulation comprising a plurality of polymeric nanoparticles to a subject in need thereof, wherein each polymeric nanoparticle is a polymeric nanoparticle according to any one of claims 1-20, and wherein the nucleic acid is an interfering RNA or a DNA encoding an interfering RNA bound by ionic-charge interactions to the polycationic polymer scaffolds, wherein the interfering RNA comprises a sequence complementary to a portion of a gene transcript for CD-44, CD-44 V6, Sox2, Id-1, Id-3, c-Met, or NCOA3.

33. A method of introducing a nucleic acid into a prostate cancer cell, the method comprising contacting the cell with a polymeric nanoparticle comprising an aggregate of nucleic acids and polycationic polymer scaffolds, wherein the aggregate comprises:
a first polycationic polymer scaffold comprising a polycationic polymer-bound Transferrin (TPL);
a hydrophilic polymer covalently bound to a second polycationic polymer scaffold;
an amphiphilic peptide covalently bound to a third polycationic polymer scaffold to provide a polycationic polymer scaffold-bound amphiphilic peptide, wherein the nanoparticle comprises greater than 6E+13 of the polycationic polymer scaffold-bound amphiphilic peptide;
a fourth polycationic polymer scaffold comprising a copolymer of a polycationic polymer and a hydrophobic polymer (PLPL);
a fifth polycationic polymer scaffold comprising a copolymer of a polycationic polymer and a polyethylenimine (LXEI); and
a nucleic acid bound by ionic-charge interactions to the polycationic polymer scaffolds.

34. The method of claim 33, wherein the hydrophilic polymer covalently bound to a second polycationic polymer scaffold is a polycationic polymer-bound PEG (PPL).

35. The method of claim 33 or 34, wherein the amphiphilic peptide covalently bound to a third polycationic polymer scaffold is a polycationic polymer-bound Am1peptide (AmPL).

36. The method of any one of claims 33-35, wherein the fourth polycationic polymer scaffold comprising a copolymer of a polycationic polymer and a hydrophobic polymer (PLPL) is a poly-lysine conjugated to lyso-phosphatidylethanolamine.

37. The method of any one of claims 33-36, wherein the nanoparticle comprises a polycationic polymer scaffold comprising a detectable label.

38. The method of claim 37, wherein the detectable label is a fluorescent label.

39. The method of claim 38, wherein the polycationic polymer scaffold comprising a detectable label is a polycationic polymer-bound Cy5.5 fluorescent label (CyPL) or polycationic polymer-bound rhodamine (RPL).

40. The method of any one of claims 33-39, wherein one or more of the polycationic polymer scaffolds comprises one or more of poly-lysine, poly-arginine, poly-glutamine, poly-amine, poly(diallydimethylammonium chloride) (pDADMAC), and chitosan.

41. The method of any one of claims 33-40, wherein the nucleic acid comprises DNA.

42. The method of claim 41, wherein the DNA encodes an interfering RNA.

43. The method of claim 42 wherein the interfering RNA comprises a short-hairpin RNA (shRNA).

44. The method of any one of claims 33-40, wherein the nucleic acid comprises RNA.

45. The method of claim 44, wherein the RNA comprises interfering RNA.

46. The method of claim 45, wherein the interfering RNA comprises a small interfering RNA (siRNA).

47. The method of claim 45, wherein the interfering RNA comprises a shRNA.

48. The method of claim 45, wherein the interfering RNA comprises a micro-RNA (miRNA).

49. A method of treating prostate cancer in a subject, the method comprising: administering a therapeutically effective amount of a formulation comprising a plurality of polymeric nanoparticles to a subject in need thereof, wherein each polymeric nanoparticle is a polymeric nanoparticle as recited in the method of any one of claims 33-48, and wherein the nucleic acid is an interfering RNA or a DNA encoding an interfering RNA bound by ionic-charge interactions to the polycationic polymer scaffolds, wherein the interfering RNA comprises a sequence complementary to a portion of a gene transcript for a gene which is upregulated in prostate cancer.

50. The method of claim 49, wherein the gene is a gene encoding a transcription factor.

51. The method of claim 49, wherein the gene is selected from CD44, PSMA, PD-L1, and PD-1.

52. A method of treating prostate cancer in a subject, the method comprising:
administering a therapeutically effective amount of a formulation comprising a plurality of polymeric nanoparticles to a subject in need thereof, wherein each polymeric nanoparticle is a polymeric nanoparticle as recited in the method of any one of claims 33-40, and wherein the nucleic acid is an interfering RNA or a DNA encoding an interfering RNA bound by ionic-charge interactions to the polycationic polymer scaffolds, wherein the interfering RNA comprises a microRNA which is downregulated in prostate cancer or which targets a gene transcript for a gene which is upregulated in prostate cancer.

53. The method of claim 52, wherein the microRNA is selected from mir-34a, mir-205, mir-18, mir-101, and mir-7.

54. The method of claim 52, wherein the microRNA is a microRNA that targets a component of the PD-L1/PD-1 pathway.

55. A method of introducing a nucleic acid into a melanoma cell, the method comprising contacting the cell with a polymeric nanoparticle comprising an aggregate of nucleic acids and polycationic polymer scaffolds, wherein the aggregate comprises:
a first polycationic polymer scaffold comprising a polycationic polymer-bound Transferrin (TPL);
a hydrophilic polymer covalently bound to a second polycationic polymer scaffold;
an amphiphilic peptide covalently bound to a third polycationic polymer scaffold;
a fourth polycationic polymer scaffold comprising a copolymer of a polycationic polymer and a hydrophobic polymer (PLPL);
a fifth polycationic polymer scaffold comprising a copolymer of a polycationic polymer and a polyethylenimine (LXEI); and
a nucleic acid bound by ionic-charge interactions to the polycationic polymer scaffolds.

56. The method of claim 55, wherein the hydrophilic polymer covalently bound to a second polycationic polymer scaffold is a polycationic polymer-bound PEG (PPL).

57. The method of claim 55 or 56, wherein the amphiphilic peptide covalently bound to a third polycationic polymer scaffold is a polycationic polymer-bound Am1peptide (AmPL).

58. The method of any one of claims 55-57, wherein the fourth polycationic polymer scaffold comprising a copolymer of a polycationic polymer and a hydrophobic polymer (PLPL) is a poly-lysine conjugated to lyso-phosphatidylethanolamine.

59. The method of any one of claims 55-58, wherein the nanoparticle comprises a polycationic polymer scaffold comprising a detectable label.

60. The method of claim 59, wherein the detectable label is a fluorescent label.

61. The method of claim 60, wherein the polycationic polymer scaffold comprising a detectable label is a polycationic polymer-bound Cy5.5 fluorescent label (CyPL) or polycationic polymer-bound rhodamine (RPL).

62. The method of any one of claims 55-61, wherein one or more of the polycationic polymer scaffolds comprises one or more of poly-lysine, poly-arginine, poly-glutamine, poly-amine, poly(diallydimethylammonium chloride) (pDADMAC), and chitosan.

63. The method of any one of claims 55-62, wherein the nucleic acid comprises DNA.

64. The method of claim 63, wherein the DNA encodes an interfering RNA.

65. The method of claim 64 wherein the interfering RNA comprises a short-hairpin RNA (shRNA).

66. The method of any one of claims 55-62, wherein the nucleic acid comprises RNA.

67. The method of claim 66, wherein the RNA comprises interfering RNA.

68. The method of claim 67, wherein the interfering RNA comprises a small interfering RNA (siRNA).

69. The method of claim 67, wherein the interfering RNA comprises a shRNA.

70. The method of claim 67, wherein the interfering RNA comprises a micro-RNA (miRNA).

71. A method of treating melanoma in a subject, the method comprising: administering a therapeutically effective amount of a formulation comprising a plurality of polymeric nanoparticles to a subject in need thereof, wherein each polymeric nanoparticle is a polymeric nanoparticle as recited in the method of any one of claims 55-70, and wherein the nucleic acid is an interfering RNA or a DNA encoding an interfering RNA bound by ionic-charge interactions to the polycationic polymer scaffolds, wherein the interfering RNA comprises a sequence complementary to a portion of a gene transcript for a gene which is upregulated in melanoma.

72. The method of claim 71, wherein the gene is selected from BPTF, CD44, a Sox gene, PD-L1 and PD-1.

73. A method of treating prostate cancer in a subject, the method comprising: administering a therapeutically effective amount of a formulation comprising a plurality of polymeric nanoparticles to a subject in need thereof, wherein each polymeric nanoparticle is a polymeric nanoparticle as recited in the method of any one of claims 55-62, and wherein the nucleic acid is an interfering RNA or a DNA encoding an interfering RNA bound by ionic-charge interactions to the polycationic polymer scaffolds, wherein the interfering RNA comprises a microRNA which is downregulated in melanoma or which targets a gene transcript for a gene which is upregulated in melanoma.

74. The method of claim 73, wherein the microRNA is selected from mir-34, mir-18, mir-7, mir-101, and mir-7.

75. The method of claim 73, wherein the microRNA is a microRNA that targets a component of the PD-L1/PD-1 pathway.


SEQUENCE LISTING (78 sequences)

SEQ ID NO: 1 - DNA, 52 bp, Artificial sequence
  synthetic nucleic acid sequence
acctcgaactggtgtcccggatattcaagagatatccgggacaccagttctt

SEQ ID NO: 2 - DNA, 52 bp, Artificial sequence
  synthetic nucleic acid sequence
caaaaagaactggtgtcccggatatctcttgaatatccgggacaccagttcg

SEQ ID NO: 3 - DNA, 52 bp, Artificial sequence
  synthetic nucleic acid sequence
acctcgaacagcgagctaaatatatcaagagtatatttagctcgctgttctt

SEQ ID NO: 4 - DNA, 52 bp, Artificial sequence
  synthetic nucleic acid sequence
caaaaagaacagcgagctaaatatactcttgatatatttagctcgctgttcg

SEQ ID NO: 5 - DNA, 62 bp, Artificial sequence
  synthetic nucleic acid sequence
gtacctcgaactggtgtcccggatattcaagagatatccgggacaccagttctttttggaaa

SEQ ID NO: 6 - DNA, 62 bp, Artificial sequence
  synthetic nucleic acid sequence
agcttttccaaaaagaactggtgtcccggatatctcttgaatatccgggacaccagttcgag

SEQ ID NO: 7 - DNA, 64 bp, Artificial sequence
  synthetic nucleic acid sequence
gtacctcgaacagcgagctaaatatattcaagagatatatttagctcgctgttctttttggaaa

SEQ ID NO: 8 - DNA, 64 bp, Artificial sequence
  synthetic nucleic acid sequence
agcttttccaaaaagaacagcgagctaaatatatctcttgaatatatttagctcgctgttcgag

SEQ ID NO: 9 - DNA, 52 bp, Artificial sequence
  synthetic nucleic acid sequence
acctcgagccagcctgaatgatgatcaagagtcatcattcaggctggctctt

SEQ ID NO: 10 - DNA, 52 bp, Artificial sequence
  synthetic nucleic acid sequence
caaaaagagccagcctgaatgatgactcttgatcatcattcaggctggctcg

SEQ ID NO: 11 - DNA, 52 bp, Artificial sequence
  synthetic nucleic acid sequence
acctcgtaagtgcccgaagtgtaatcaagagttacacttcgggcacttactt

SEQ ID NO: 12 - DNA, 52 bp, Artificial sequence
  synthetic nucleic acid sequence
caaaaagtaagtgcccgaagtgtaactcttgattacacttcgggcacttacg

SEQ ID NO: 13 - DNA, 62 bp, Artificial sequence
  synthetic nucleic acid sequence
gtacctcgagccagcctgaatgatgatcaagagtcatcattcaggctggctctttttggaaa

SEQ ID NO: 14 - DNA, 62 bp, Artificial sequence
  synthetic nucleic acid sequence
agcttttccaaaaagagccagcctgaatgatgactcttgatcatcattcaggctggctcgag

SEQ ID NO: 15 - DNA, 62 bp, Artificial sequence
  synthetic nucleic acid sequence
gtacctcgtaagtgcccgaagtgtaatcaagagttacacttcgggcacttactttttggaaa

SEQ ID NO: 16 - DNA, 62 bp, Artificial sequence
  synthetic nucleic acid sequence
agcttttccaaaaagtaagtgcccgaagtgtaactcttgattacacttcgggcacttacgag

SEQ ID NO: 17 - DNA, 66 bp, Artificial sequence
  synthetic nucleic acid sequence
gtacctcgcaggtaaacgtgctgctctatcaagagtagagcagcacgtttacctgctttttggaaa

SEQ ID NO: 18 - DNA, 66 bp, Artificial sequence
  synthetic nucleic acid sequence
agcttttccaaaaagcaggtaaacgtgctgctctactcttgatagagcagcacgtttacctgcgag

SEQ ID NO: 19 - DNA, 56 bp, Artificial sequence
  synthetic nucleic acid sequence
acctcgcaggtaaacgtgctgctctatcaagagtagagcagcacgtttacctgctt

SEQ ID NO: 20 - DNA, 56 bp, Artificial sequence
  synthetic nucleic acid sequence
caaaaagcaggtaaacgtgctgctctactcttgatagagcagcacgtttacctgcg

SEQ ID NO: 21 - DNA, 65 bp, Artificial sequence
  synthetic nucleic acid sequence
gtacctccctactagtcaccagagacttctcgagaagtctctggtgactagtaggtttttggaaa

SEQ ID NO: 22 - DNA, 65 bp, Artificial sequence
  synthetic nucleic acid sequence
agcttttccaaaaacctactagtcaccagagacttctcgagaagtctctggtgactagtagggag

SEQ ID NO: 23 - DNA, 55 bp, Artificial sequence
  synthetic nucleic acid sequence
acctccctactagtcaccagagacttctcgagaagtctctggtgactagtaggtt

SEQ ID NO: 24 - DNA, 55 bp, Artificial sequence
  synthetic nucleic acid sequence
caaaaacctactagtcaccagagacttctcgagaagtctctggtgactagtaggg

SEQ ID NO: 25 - DNA, 52 bp, Artificial sequence
  synthetic nucleic acid sequence
acctcgcgcagatcgatttgaatatcaagagtattcaaatcgatctgcgctt

SEQ ID NO: 26 - DNA, 52 bp, Artificial sequence
  synthetic nucleic acid sequence
caaaaagcgcagatcgatttgaatactcttgatattcaaatcgatctgcgcg

SEQ ID NO: 27 - DNA, 62 bp, Artificial sequence
  synthetic nucleic acid sequence
gtacctcgcgcagatcgatttgaatatcaagagtattcaaatcgatctgcgctttttggaaa

SEQ ID NO: 28 - DNA, 62 bp, Artificial sequence
  synthetic nucleic acid sequence
agcttttccaaaaagcgcagatcgatttgaatactcttgatattcaaatcgatctgcgcgag

SEQ ID NO: 29 - DNA, 52 bp, Artificial sequence
  synthetic nucleic acid sequence
acctcgcaactcctagtagtacaatcaagagttgtactactaggagttgctt

SEQ ID NO: 30 - DNA, 52 bp, Artificial sequence
  synthetic nucleic acid sequence
caaaaagcaactcctagtagtacaactcttgattgtactactaggagttgcg

SEQ ID NO: 31 - DNA, 62 bp, Artificial sequence
  synthetic nucleic acid sequence
gtacctcgcaactcctagtagtacaatcaagagttgtactactaggagttgctttttggaaa

SEQ ID NO: 32 - DNA, 62 bp, Artificial sequence
  synthetic nucleic acid sequence
agcttttccaaaaagcaactcctagtagtacaactcttgattgtactactaggagttgcgag

SEQ ID NO: 33 - DNA, 52 bp, Artificial sequence
  synthetic nucleic acid sequence
acctctgagggatatcgccaaacatcaagagtgtttggcgatatccctcatt

SEQ ID NO: 34 - DNA, 52 bp, Artificial sequence
  synthetic nucleic acid sequence
caaaaatgagggatatcgccaaacactcttgatgtttggcgatatccctcag

SEQ ID NO: 35 - DNA, 62 bp, Artificial sequence
  synthetic nucleic acid sequence
gtacctctgagggatatcgccaaacatcaagagtgtttggcgatatccctcatttttggaaa

SEQ ID NO: 36 - DNA, 62 bp, Artificial sequence
  synthetic nucleic acid sequence
agcttttccaaaaatgagggatatcgccaaacactcttgatgtttggcgatatccctcagag

SEQ ID NO: 37 - DNA, 52 bp, Artificial sequence
  synthetic nucleic acid sequence
acctcggcgcagatcgatttgaattcaagagattcaaatcgatctgcgcctt

SEQ ID NO: 38 - DNA, 52 bp, Artificial sequence
  synthetic nucleic acid sequence
caaaaaggcgcagatcgatttgaatctcttgaattcaaatcgatctgcgccg

SEQ ID NO: 39 - DNA, 62 bp, Artificial sequence
  synthetic nucleic acid sequence
gtacctcggcgcagatcgatttgaattcaagagattcaaatcgatctgcgcctttttggaaa

SEQ ID NO: 40 - DNA, 62 bp, Artificial sequence
  synthetic nucleic acid sequence
agcttttccaaaaaggcgcagatcgatttgaatctcttgaattcaaatcgatctgcgccgag

SEQ ID NO: 41 - DNA, 72 bp, Artificial sequence
  synthetic nucleic acid sequence
acctcactcagcttagccaggtggaaatcctacatcaagagtgtaggatttccacctggctaagctgagttt

SEQ ID NO: 42 - DNA, 72 bp, Artificial sequence
  synthetic nucleic acid sequence
caaaaaactcagcttagccaggtggaaatcctacactcttgatgtaggatttccacctggctaagctgagtg

SEQ ID NO: 43 - DNA, 82 bp, Artificial sequence
  synthetic nucleic acid sequence
gtacctcactcagcttagccaggtggaaatcctacatcaagagtgtaggatttccacctggctaagctgagttttttggaaa

SEQ ID NO: 44 - DNA, 82 bp, Artificial sequence
  synthetic nucleic acid sequence
agcttttccaaaaaactcagcttagccaggtggaaatcctacactcttgatgtaggatttccacctggctaagctgagtgag

SEQ ID NO: 45 - DNA, 72 bp, Artificial sequence
  synthetic nucleic acid sequence
acctcatcgactacattctcgacctgcaggtagttcaagagactacctgcaggtcgagaatgtagtcgattt

SEQ ID NO: 46 - DNA, 72 bp, Artificial sequence
  synthetic nucleic acid sequence
caaaaaatcgactacattctcgacctgcaggtagtctcttgaactacctgcaggtcgagaatgtagtcgatg

SEQ ID NO: 47 - DNA, 82 bp, Artificial sequence
  synthetic nucleic acid sequence
gtacctcatcgactacattctcgacctgcaggtagttcaagagactacctgcaggtcgagaatgtagtcgattttttggaaa

SEQ ID NO: 48 - DNA, 82 bp, Artificial sequence
  synthetic nucleic acid sequence
agcttttccaaaaaatcgactacattctcgacctgcaggtagtctcttgaactacctgcaggtcgagaatgtagtcgatgag

SEQ ID NO: 49 - DNA, 72 bp, Artificial sequence
  synthetic nucleic acid sequence
acctcaccttcccatccagacagccgagctcacttcaagagagtgagctcggctgtctggatgggaaggttt

SEQ ID NO: 50 - DNA, 72 bp, Artificial sequence
  synthetic nucleic acid sequence
caaaaaaccttcccatccagacagccgagctcactctcttgaagtgagctcggctgtctggatgggaaggtg

SEQ ID NO: 51 - DNA, 82 bp, Artificial sequence
  synthetic nucleic acid sequence
gtacctcaccttcccatccagacagccgagctcacttcaagagagtgagctcggctgtctggatgggaaggttttttggaaa

SEQ ID NO: 52 - DNA, 82 bp, Artificial sequence
  synthetic nucleic acid sequence
agcttttccaaaaaaccttcccatccagacagccgagctcactctcttgaagtgagctcggctgtctggatgggaaggtgag

SEQ ID NO: 53 - DNA, 72 bp, Artificial sequence
  synthetic nucleic acid sequence
acctcccggaacttgtcatctccaacgacaaaagtcaagagcttttgtcgttggagatgacaagttccggtt

SEQ ID NO: 54 - DNA, 72 bp, Artificial sequence
  synthetic nucleic acid sequence
caaaaaccggaacttgtcatctccaacgacaaaagctcttgacttttgtcgttggagatgacaagttccggg

SEQ ID NO: 55 - DNA, 82 bp, Artificial sequence
  synthetic nucleic acid sequence
gtacctcccggaacttgtcatctccaacgacaaaagtcaagagcttttgtcgttggagatgacaagttccggtttttggaaa

SEQ ID NO: 56 - DNA, 82 bp, Artificial sequence
  synthetic nucleic acid sequence
agcttttccaaaaaccggaacttgtcatctccaacgacaaaagctcttgacttttgtcgttggagatgacaagttccgggag

SEQ ID NO: 57 - DNA, 56 bp, Artificial sequence
  synthetic nucleic acid sequence
acctcgcagtctattcgtcctccatatcaagagtatggaggacgaatagactgctt

SEQ ID NO: 58 - DNA, 56 bp, Artificial sequence
  synthetic nucleic acid sequence
caaaaagcagtctattcgtcctccatactcttgatatggaggacgaatagactgcg

SEQ ID NO: 59 - DNA, 66 bp, Artificial sequence
  synthetic nucleic acid sequence
gtacctcgcagtctattcgtcctccatatcaagagtatggaggacgaatagactgctttttggaaa

SEQ ID NO: 60 - DNA, 66 bp, Artificial sequence
  synthetic nucleic acid sequence
agcttttccaaaaagcagtctattcgtcctccatactcttgatatggaggacgaatagactgcgag

SEQ ID NO: 61 - protein, 36 aa, Leiurus quinquestriatus quinquestriatus
MCMPCFTTDHQMARKCDDCCGGKGRGKCYGPQCLCR

SEQ ID NO: 62 - protein, 27 aa, Artificial sequence
  synthetic amino acid sequence
GIGAVLKVLTTGLPALISWIKRKRHHC

SEQ ID NO: 63 - RNA, 25 nt, Artificial sequence
  synthetic nucleic acid sequence
cauaauaucgucuuuguacucuaaa

SEQ ID NO: 64 - RNA, 25 nt, Artificial sequence
  synthetic nucleic acid sequence
agaguacaaagacgauauuaugaaa

SEQ ID NO: 65 - DNA, 21 bp, Artificial sequence
  synthetic nucleic acid sequence
gcgcagaucgauuugaauatt

SEQ ID NO: 66 - RNA, 21 nt, Artificial sequence
  synthetic nucleic acid sequence
uauucaaaucgaucugcgcca

SEQ ID NO: 67 - RNA, 21 nt, Artificial sequence
  synthetic nucleic acid sequence
uggucauggaguucuacugca

SEQ ID NO: 68 - RNA, 21 nt, Artificial sequence
  synthetic nucleic acid sequence
caguacaacuccaugaccacg

SEQ ID NO: 69 - DNA, 21 bp, Artificial sequence
  synthetic nucleic acid sequence
uucauguaggucugcgagctg

SEQ ID NO: 70 - DNA, 21 bp, Artificial sequence
  synthetic nucleic acid sequence
gcucgcagacctacatgaacg

SEQ ID NO: 71 - RNA, 21 nt, Artificial sequence
  synthetic nucleic acid sequence
uacuuauccuucuucaugagc

SEQ ID NO: 72 - RNA, 21 nt, Artificial sequence
  synthetic nucleic acid sequence
ucaugaagaaggauaaguaca

SEQ ID NO: 73 - RNA, 21 nt, Artificial sequence
  synthetic nucleic acid sequence
aacccauggagccaagagcca

SEQ ID NO: 74 - RNA, 21 nt, Artificial sequence
  synthetic nucleic acid sequence
gcucuuggcuccauggguucg

SEQ ID NO: 75 - RNA, 22 nt, Homo sapiens
uggcagugucuuagcugguugu

SEQ ID NO: 76 - RNA, 110 nt, Homo sapiens
ggccagcugugaguguuucuuuggcagugucuuagcugguuguugugagcaauaguaaggaagcaaucagcaaguauacugcccuagaagugcugcacguuguggggccc

SEQ ID NO: 77 - RNA, 23 nt, Homo sapiens
gggggccgauacacuguacgaga

SEQ ID NO: 78 - RNA, 84 nt, Homo sapiens
ugugcagugggaaggggggccgauacacuguacgagagugaguagcaggucucacagugaaccggucucuuucccuacuguguc